(19) United States
(12) Patent Application Publication
(10) Pub. No.: US 2023/0212174 A1
(43) Pub. Date: Jul. 6, 2023

(54) PYRROLO[2,1-F][1,2,4]TRIAZINE DERIVATIVE AND USE THEREOF

(71) Applicant: Tencent Technology (Shenzhen) Company Limited, Shenzhen (CN)

(72) Inventor: Xinde CHEN, Shenzhen (CN)

(21) Appl. No.: 18/122,465

(22) Filed: Mar. 16, 2023

Related U.S. Application Data
(63) Continuation of application No. PCT/CN2022/094068, filed on May 20, 2022.

(30) Foreign Application Priority Data
Jun. 11, 2021 (CN) 202110653809.4

(51) Int. Cl. C07D 487/04; A61K 45/06

(52) U.S. Cl. CPC C07D 487/04; A61K 45/06

(57) ABSTRACT

The present disclosure provides a compound, which is a compound of Formula (I) or a stereoisomer, a tautomer, a N-oxide, a solvate, a metabolite, a pharmaceutically acceptable salt or a prodrug of the compound of Formula (I):

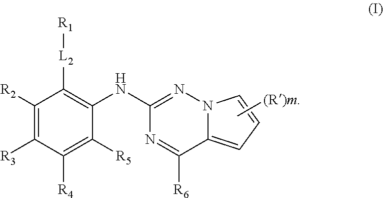

### CROSS-REFERENCE TO RELATED APPLICATION

This application is a continuation application of PCT Patent Application No. PCT/CN2022/094068, entitled “PYRROLO[2,1-F][1,2,4]TRIAZINE DERIVATIVE AND USE THEREOF” filed on May 20, 2022, which claims priority to and benefit of Chinese Patent Application No. 202110653809.4, entitled “PYRROLO[2,1-F][1,2,4]TRIAZINE DERIVATIVE AND USE THEREOF” filed on Jun. 11, 2021 with the State Intellectual Property Office, all of which are incorporated herein by reference in their entirety.

### FIELD OF THE TECHNOLOGY

The present disclosure relates to the field of medicines, and in particular to a pyrrolo[2,1-f] [1,2,4] triazine derivative and use thereof.

### BACKGROUND OF THE DISCLOSURE

Spindle assembly checkpoint (SAC) is one of the main checkpoints in the cell cycle. SAC monitors the arrangement of chromosomes on the equatorial plate and the separation to the spindle poles, and ensures the attachment of kinetome-microtubule and the completeness of mitosis, so that all chromosomes are positioned on the equatorial plate and bi-oriented before entering the later stage, thus ensuring the accurate allocation of chromosomes to daughter cells during mitosis. When the spindle microtubule is incorrectly attached with the chromosomes or errors occur in the assembly of spindle, SAC is activated to arrest the progression of cell cycle. Over-expression or non-expression of SAC members has been reported in various types of cancers. In most cases, the expression status of SAC members is associated to high proliferative activity and poor prognosis of tumors.

Threonine and tyrosine kinase (TTK), also known as monopolar spindle 1 (Mps1) is a key kinase in the activation and maintenance of SAC function. Except in testis and placenta, TTK is almost undetectable in normal tissues. However, TTK mRNA levels are increased in many human cancers, including papillary thyroid carcinoma, breast cancer, gastric cancer, bronchial cancer and lung cancer. Inhibition of TTK can lead to SAC deficiency, causing premature mitosis interruption, seriously incorrect separation of chromosomes, and finally death of cancer cells.

Therefore, TTK inhibitors have great potential in the treatment of tumors.

### SUMMARY

The present disclosure relates to a pyrrolo[2,1-f][1,2,4]triazine derivative, which shows potent inhibitory activity on TTK in biological activity tests in vitro, and has the potential to be used as a novel TTK inhibitor in the treatment of tumors.

The present disclosure provides a compound, which is a compound of Formula (I) or a stereoisomer, a tautomer, a N-oxide, a solvate, a metabolite, a pharmaceutically acceptable salt or a prodrug of the compound of Formula (I):

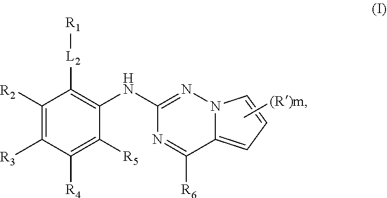

where R₁, R₂, R₃, R₄, R₅, R₆, L₂, R′ and m have the meanings as defined herein.

In some other embodiments, L₂ is a bond or O;

R₁, R₂, R₄, and R₅ are each independently H, F, Cl, Br, CN, NO₂, —OR^{b}, —NR^{c}R^{d}, or C₁₋₆ alkyl;

R₃ is —C(═O)R^{a}, —C(═O)OR^{b}, —S(═O)₂R^{b}, —C(═O)NR^{c}R^{d}, —OR^{b}, —NR^{c}R^{d}, R^{b}O—C₁₋₄ alkylene, R^{d}R^{c}N—C₁₋₄ alkylene, C₁₋₆ alkyl, C₂₋₆ alkenyl, C₂₋₆ alkynyl, C₃₋₈ cycloalkyl, C₃₋₈ cycloalkyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 10 atoms, or (heteroaryl having 5 to 10 atoms)-C₁₋₄ alkylene, in which the C₁₋₆ alkyl, C₂₋₆ alkenyl, C₂₋₆ alkynyl, C₃₋₈ cycloalkyl, C₃₋₈ cycloalkyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 10 atoms and (heteroaryl having 5 to 10 atoms)-C₁₋₄ alkylene are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene, or R^{d}R^{c}N—C₁₋₄ alkylene;

R₆ is H or

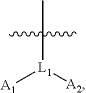

in which L₁ is N or O;

A₁ and A₂ are each independently H, C₁₋₆ alkyl, C₃₋₁₂ carbocyclyl, C₃₋₁₂ carbocyclyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 14 atoms, or (heteroaryl having 5 to 14 atoms)-C₁₋₄ alkylene, or A₁ and A₂, together with L₁ to which they are attached, form a heterocyclic ring having 3-6 atoms, in which the C₁₋₆ alkyl, C₃₋₁₂ carbocyclyl, C₃₋₁₂ carbocyclyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 14 atoms, (heteroaryl having 5 to 14 atoms)-C₁₋₄ alkylene, or heterocyclic ring having 3-6 atoms formed by A₁ and A₂ together with L₁ to which they are attached are each independently unsubstituted or substituted with 1, 2, 3, 4 or 5 R′, provided that A₁ and A₂ are not both H;

each R′ is independently H, F, Cl, Br, CN, NO₂, ═O, —OR^{b}, —NR^{c}R^{d}, —S(═O)₂R^{b}, C₁₋₆ alkyl|, C₁₋₆ haloalkyl, C₃₋₈ cycloalkyl, C₃₋₈ cycloalkenyl, heterocyclyl having 3 to 12 atoms, C₆₋₁₀ aryl or heteroaryl having 5 to 10 atoms, in which the C₃₋₈ cycloalkyl, heterocyclyl having 3 to 12 atoms, C₆₋₁₀ aryl or heteroaryl having 5 to 10 atoms are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene;

m is 0, 1, 2 or 3;

provided that when R₆ is H, m is not 0, and at least one R′ is C₃₋₈ cycloalkyl, C₃₋₈ cycloalkenyl, heterocyclyl having 3 to 12 atoms, C₆₋₁₀ aryl or heteroaryl having 5 to 10 atoms, in which the C₃₋₈ cycloalkyl, C₃₋₈ cycloalkenyl, heterocyclyl having 3 to 12 atoms, C₆₋₁₀ aryl heteroaryl having 5 to 10 atoms are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene;

R^{a}, R^{b}, R^{c}, and R^{d} are each independently H, C₁₋₆ alkyl, C₁₋₆ haloalkyl, or heterocyclyl having 3 to 6 atoms, or R^{c} and R^{d}, together with nitrogen to which they are attached, form a heterocyclic ring having 3 to 6 atoms, in which the C₁₋₆ alkyl and heterocyclic ring having 3 to 6 atoms are each independently unsubstituted or substituted with 1, 2, 3 or 4 substituents independently selected from F, Cl, CN, OH, NH₂, C₁₋₆ alkyl, C₁₋₆ haloalkyl, C₁₋₆ alkoxy or C₁₋₆ alkylamino.

In some other embodiments, the present disclosure relates to a compound of Formula (II) or a stereoisomer, a tautomer, a N-oxide, a solvate, a metabolite, a pharmaceutically acceptable salt or a prodrug of the compound of Formula (II):

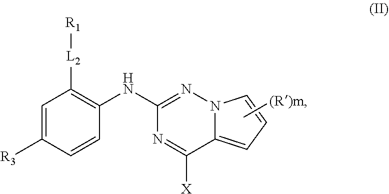

where R₁, R₃, L₂, R′ and m have the meanings as defined herein.

In some other embodiments, X is represented by a sub-structural formula below:

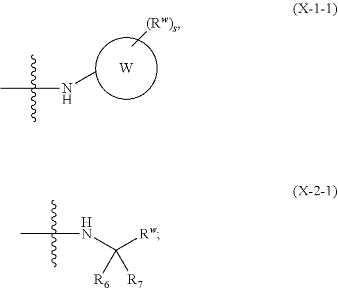

where ring W is C₃₋₈ cycloalkyl, a heterocyclic ring having 3 to 8 atoms, benzene or a heteroaryl ring having 5 to 6 atoms; each R^{w} is independently H, F, Cl, Br, CN, NO₂, ═O, —OR^{b}, —NR^{c}R^{d}, —S(═O)₂R^{b}, C₁₋₆ alkyl or C₁₋₆ haloalkyl; R₇ and R₈ are each independently H, or C₁₋₆ alkyl; and s is 0, 1, 2 or 3.

In some other embodiments, the present disclosure relates to a compound of Formula (III) or a stereoisomer, a tautomer, a N-oxide, a solvate, a metabolite, a pharmaceutically acceptable salt or a prodrug of the compound of Formula (III):

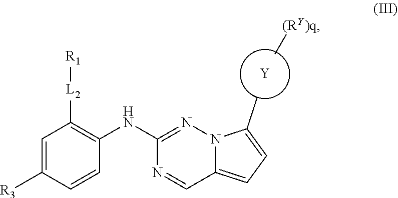

where R₁, R₃, L₂, Y, R^{Y} and q have the meanings as defined herein.

In some other embodiments, ring Y is C₃₋₈ cycloalkyl, C₃₋₈ cycloalkenyl, a heterocyclic ring having 3 to 8 atoms, benzene or a heteroaryl ring having 5 to 6 atoms; each R^{Y} is independently H, F, Cl, Br, CN, NO₂, ═O, —OR^{b}, —NR^{c}R^{d}, —S(═O)₂R^{b}, C₁₋₆ alkyl or C₁₋₆ haloalkyl; and q is 0, 1, 2 or 3.

In some other embodiments, R₂, R₄, and R₅ are each independently H.

In some other embodiments, R₃ is —C(═O)NR^{c}R^{d}, OR^{b}, —NR^{c}R^{d}, —S(═O)₂R^{b}, heterocyclyl having 3 to 6 atoms, (heterocyclyl having 3 to 6 atoms)-C₁₋₄ alkylene, C₆₋₉ aryl, C₆₋₉ aryl-C₁₋₄ alkylene, heteroaryl having 5 to 9 atoms or (heteroaryl having 5 to 9 atoms)-C₁₋₄ alkylene.

In some other embodiments, A₁ and A₂ are each independently H, C₁₋₆ alkyl, C₃₋₆ carbocyclyl, C₃₋₆ carbocyclyl-C₁₋₄ alkylene, heterocyclyl having 3 to 6 atoms, (heterocyclyl having 3 to 6 atoms)-C₁₋₄ alkylene, C₆₋₈ aryl, C₆₋₈ aryl-C₁₋₄ alkylene, heteroaryl having 5 to 8 atoms, or (heteroaryl having 5 to 8 atoms)-C₁₋₄ alkylene, or A₁ and A₂, together with L₁ to which they are attached, form a heterocyclic ring having 3 to 6 atoms.

In some other embodiments, each R′ is independently H, F, Cl, Br, CN, NO₂, ═O, —OR_{b}, —NR_{c}R_{d}, —S(═O)OR^{b}, C₁₋₃ alkyl, C₁₋₃ haloalkyl, C₃₋₆ cycloalkyl, heterocyclyl having 3 to 6 atoms, C₆₋₈ aryl or heteroaryl having 5 to 8 atoms, in which the C₃₋₆ cycloalkyl, heterocyclyl having 3 to 6 atoms, C₆₋₈ aryl or heteroaryl having 5 to 8 atoms are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene.

In some other embodiments, R^{a}, R^{b}, R^{c}, and R^{d} are each independently H, methyl, ethyl, isopropyl, n-propyl, n-butyl, t-butyl, C₁₋₃ haloalkyl, or heterocyclyl having 3 to 6 atoms, or R^{c} and R^{d}, together with nitrogen to which they are attached, form a heterocyclic ring having 3 to 6 atoms.

In some other embodiments, A₁ and A₂ are each independently H, C₁₋₆ alkyl, C₃₋₆ carbocyclyl, heterocyclyl having 3 to 6 atoms, C₆₋₈ aryl, or heteroaryl having 5 to 8 atoms.

In some other embodiments, R₃ is —C(═O)NR^{c}R^{d}, OR^{b}, —NR^{c}R^{d}, —S(═O)₂R^{b}, heterocyclyl having 3 to 6 atoms, phenyl, naphthyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, pyrimidinyl, indolyl, purinyl, quinolyl, isoquinolyl, or phenoxathiinyl, in which the heterocyclyl having 3 to 6 atoms, phenyl, naphthyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, pyrimidinyl, indolyl, purinyl, quinolyl, isoquinolyl, or phenoxathiinyl are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene.

In some other embodiments, ring W is C₃₋₆ cycloalkyl, heterocyclyl having 3 to 6 atoms, phenyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, or pyrimidinyl.

In some other embodiments, R^{w} is independently H, F, Cl, Br, CN, NO₂, ═O, —OR^{b}, —NR^{c}R^{d}, —S(═O)₂R^{b}, methyl, ethyl, isopropyl, n-propyl, n-butyl or t-butyl or C₁₋₆ haloalkyl.

In some other embodiments, ring Y is C₃₋₆ cycloalkyl, C₃₋₆ cycloalkenyl, heterocyclyl having 3 to 6 atoms, phenyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, pyrimidinyl, 3,6-dihydro-2H-pyran or tetrahydro-2H-pyran.

In some other embodiments, R^{Y} is independently H, F, Cl, Br, CN, NO₂, ═O, —OR^{b}, —NR^{c}R^{d}, —S(═O)₂R^{b}, methyl, ethyl, isopropyl, n-propyl, n-butyl or t-butyl or C₁₋₆ haloalkyl.

In some other embodiments, the present disclosure relates to a compound of Formula (IV) or a stereoisomer, a tautomer, a N-oxide, a solvate, a metabolite, a pharmaceutically acceptable salt or a prodrug of the compound of Formula (IV):

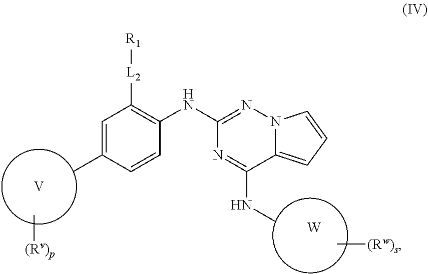

where ring V, ring W, R₁, L₂, R′, R^{w}, R^{V}, s, p, and m have the meanings as defined herein.

In some other embodiments, the present disclosure relates to a compound of Formula (V) or a stereoisomer, a tautomer, a N-oxide, a solvate, a metabolite, a pharmaceutically acceptable salt or a prodrug of the compound of Formula (V):

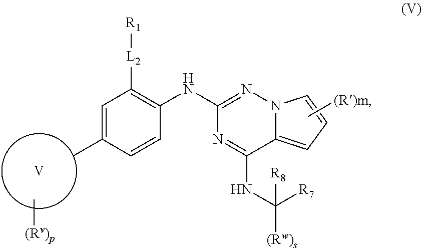

where ring V, R₁, L₂, R′, R^{w}, R^{V}, s, p, and m have the meanings as defined herein, and R₇ and R₈ are each independently H, or C₁₋₆ alkyl.

In some other embodiments, the present disclosure relates to a compound of Formula (VI) or a stereoisomer, a tautomer, a N-oxide, a solvate, a metabolite, a pharmaceutically acceptable salt or a prodrug of the compound of Formula (VI):

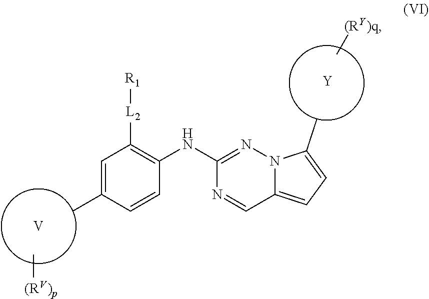

where ring V, ring Y, R₁, L₂, R^{V}, R^{V}, p, and q have the meanings as defined herein.

In some other embodiments, the present disclosure relates to a compound having a structure of Formula (VII) or a stereoisomer, a tautomer, a N-oxide, a solvate, a metabolite, a pharmaceutically acceptable salt or a prodrug of the compound of Formula (VII):

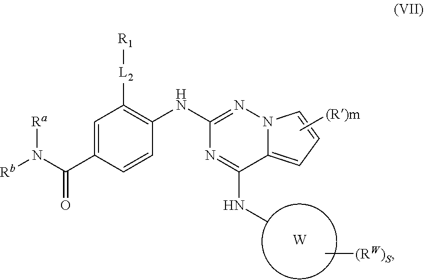

where ring W, R₁, R^{a}, R^{b}, L₂, R′, R^{w}, s, and m have the meanings as defined herein.

In some other embodiments, the present disclosure relates to a compound of Formula (VIII) or a stereoisomer, a tautomer, a N-oxide, a solvate, a metabolite, a pharmaceutically acceptable salt or a prodrug of the compound of Formula (VIII):

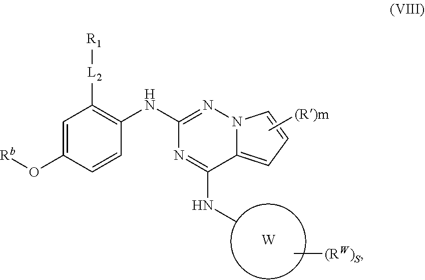

where ring W, R₁, R^{b}, L₂, R′, R^{w}, s, and m have the meanings as defined herein.

In some other embodiments, the present disclosure relates to a compound having a structure of Formula (VIIII) or a stereoisomer, a tautomer, a N-oxide, a solvate, a metabolite, a pharmaceutically acceptable salt or a prodrug of the compound of Formula (VIIII):

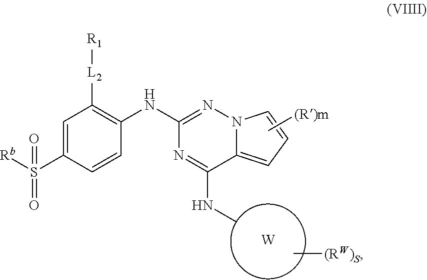

where ring W, R₁, R^{b}, L₂, R′, R^{w}, s, and m have the meanings as defined herein.

In some other embodiments, ring V is C₃₋₈ cycloalkyl, heterocyclic ring having 3 to 8 atoms, benzene or heteroaryl ring having 5 to 6 atoms; R^{V} is F, Cl, Br, CN, —OH, ═O, C₁₋₆ alkyl or C₁₋₆ haloalkyl; and p is 0, 1, 2 or 3.

In some other embodiments, the present disclosure relates to a compound having one of the structures below, or a stereoisomer, a tautomer, a N-oxide, a solvate, a metabolite, a pharmaceutically acceptable salt or a prodrug of the compound having one of the structures below.

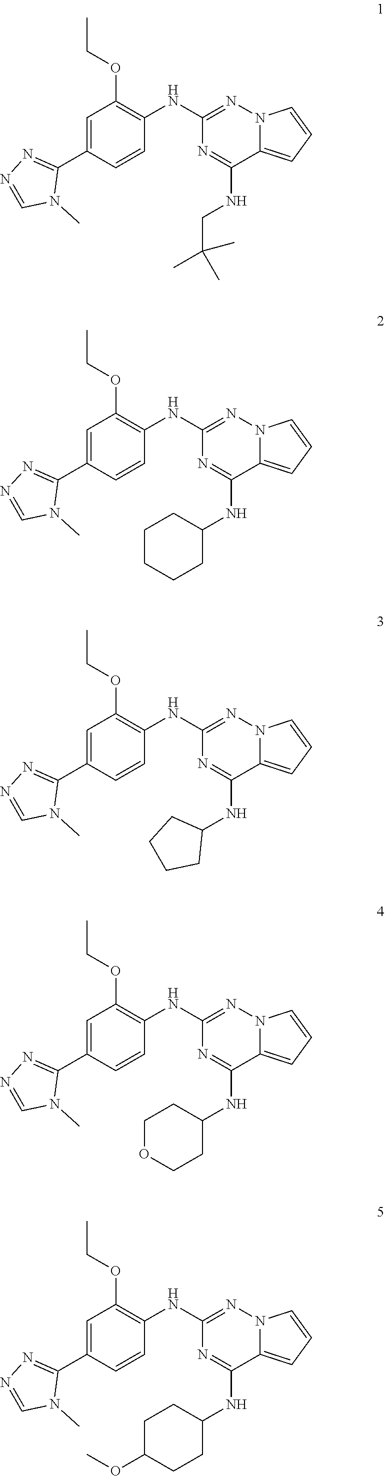 [SMILES: CCOc1cc(-c2nncn2C)ccc1Nc1nc(NCC(C)(C)C)c2cccn2n1 ; CCOc1cc(-c2nncn2C)ccc1Nc1nc(NC2CCCCC2)c2cccn2n1 ; CCOc1cc(-c2nncn2C)ccc1Nc1nc(NC2CCCC2)c2cccn2n1 ; CCOc1cc(-c2nncn2C)ccc1Nc1nc(NC2CCOCC2)c2cccn2n1 ; CCOc1cc(-c2nncn2C)ccc1Nc1nc(NC2CCC(OC)CC2)c2cccn2n1]

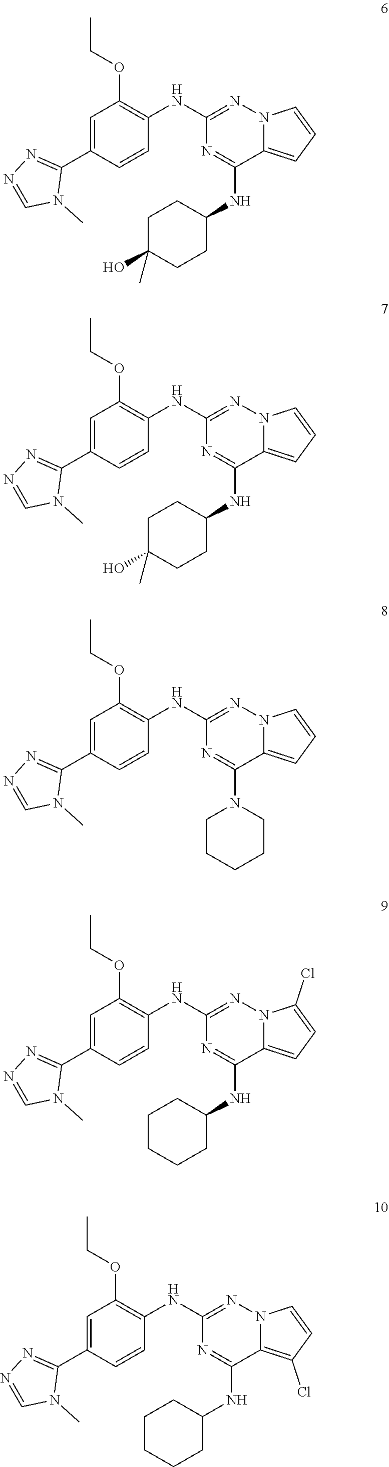

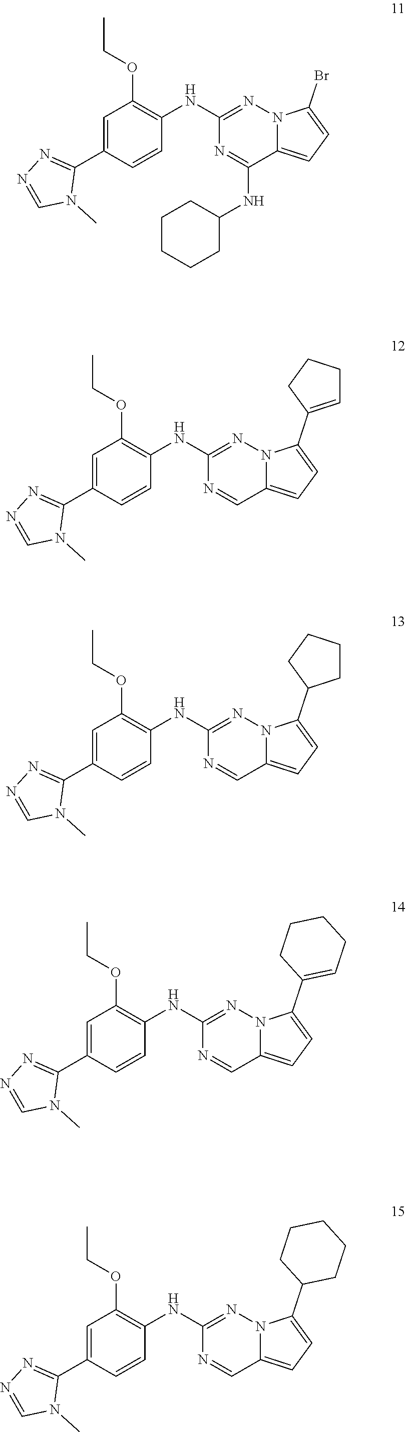 [SMILES: CCOc1cc(-c2nncn2C)ccc1Nc1nc(NC2CCCCC2)c2ccc(Br)n2n1 ; CCOc1cc(-c2nncn2C)ccc1Nc1ncc2ccc(C3=CCCC3)n2n1 ; CCOc1cc(-c2nncn2C)ccc1Nc1ncc2ccc(C3CCCC3)n2n1 ; CCOc1cc(-c2nncn2C)ccc1Nc1ncc2ccc(C3=CCCCC3)n2n1 ; CCOc1cc(-c2nncn2C)ccc1Nc1ncc2ccc(C3CCCCC3)n2n1]

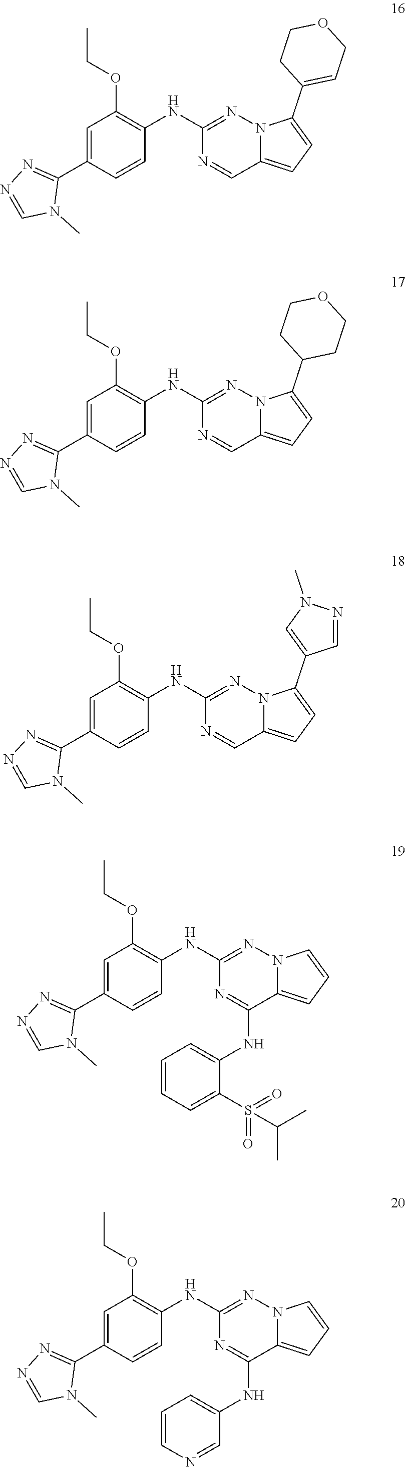 [SMILES: CCOc1cc(-c2nncn2C)ccc1Nc1ncc2ccc(C3=CCOCC3)n2n1 ; CCOc1cc(-c2nncn2C)ccc1Nc1ncc2ccc(C3CCOCC3)n2n1 ; CCOc1cc(-c2nncn2C)ccc1Nc1ncc2ccc(-c3cnn(C)c3)n2n1 ; CCOc1cc(-c2nncn2C)ccc1Nc1nc(Nc2ccccc2S(=O)(=O)C(C)C)c2cccn2n1 ; CCOc1cc(-c2nncn2C)ccc1Nc1nc(Nc2cccnc2)c2cccn2n1]

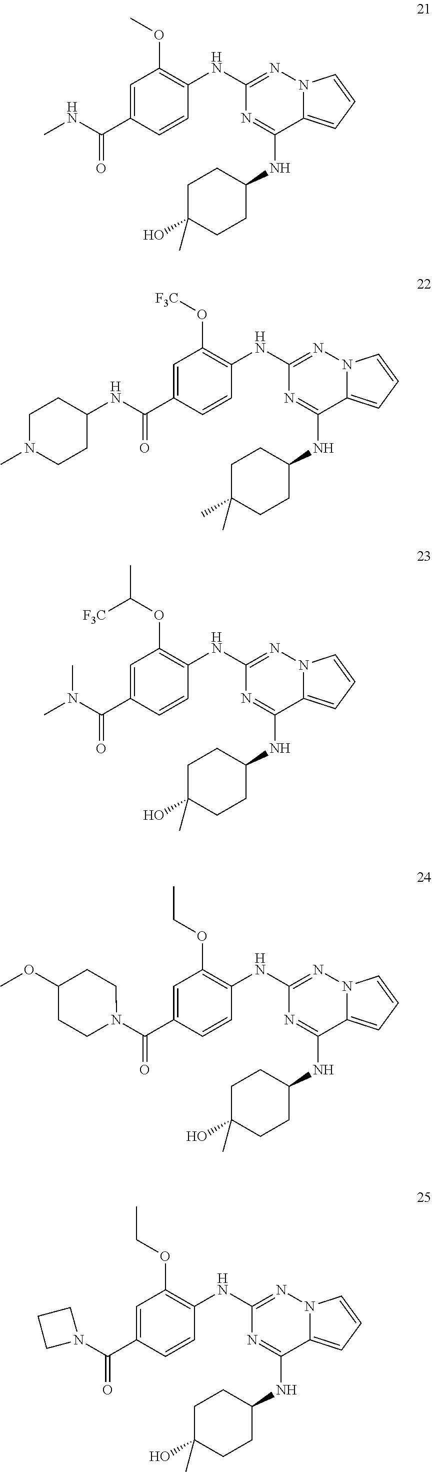

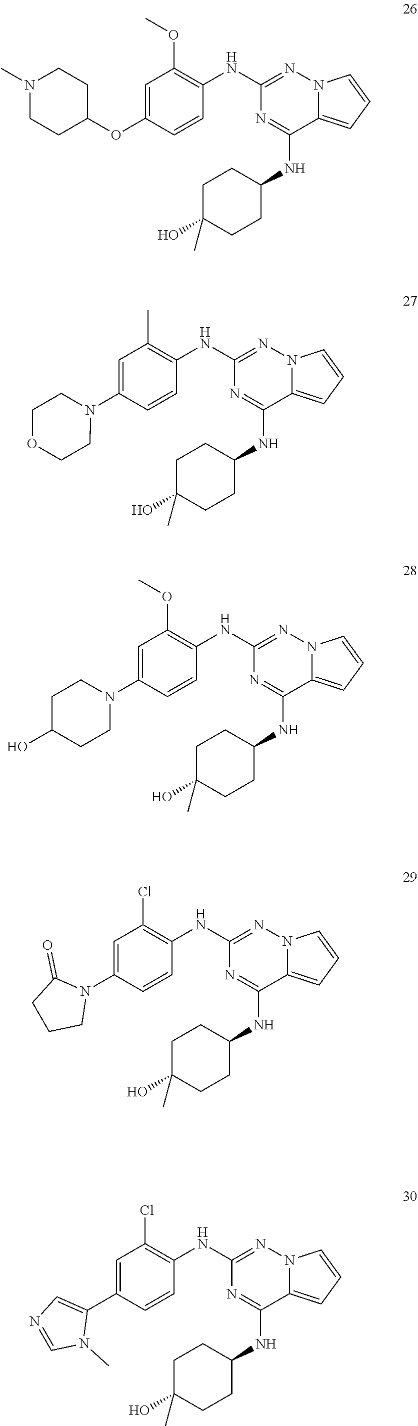 [SMILES: COc1cc(OC2CCN(C)CC2)ccc1Nc1nc(N[C@H]2CC[C@](C)(O)CC2)c2cccn2n1 ; Cc1cc(N2CCOCC2)ccc1Nc1nc(N[C@H]2CC[C@](C)(O)CC2)c2cccn2n1 ; COc1cc(N2CCC(O)CC2)ccc1Nc1nc(N[C@H]2CC[C@](C)(O)CC2)c2cccn2n1 ; C[C@]1(O)CC[C@H](Nc2nc(Nc3ccc(N4CCCC4=O)cc3Cl)nn3cccc23)CC1 ; Cn1cncc1-c1ccc(Nc2nc(N[C@H]3CC[C@](C)(O)CC3)c3cccn3n2)c(Cl)c1]

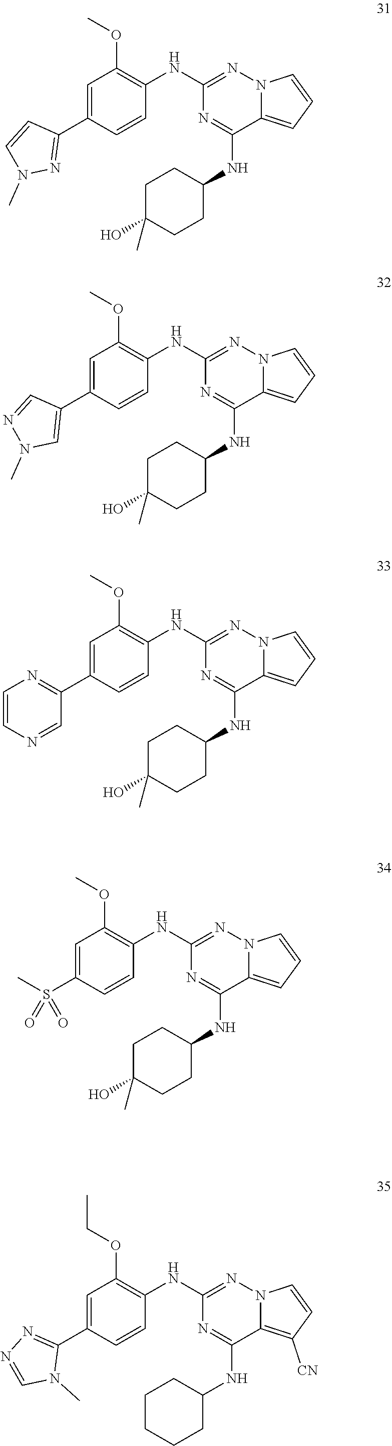 [SMILES: COc1cc(-c2ccn(C)n2)ccc1Nc1nc(N[C@H]2CC[C@](C)(O)CC2)c2cccn2n1 ; COc1cc(-c2cnn(C)c2)ccc1Nc1nc(N[C@H]2CC[C@](C)(O)CC2)c2cccn2n1 ; COc1cc(-c2cnccn2)ccc1Nc1nc(N[C@H]2CC[C@](C)(O)CC2)c2cccn2n1 ; COc1cc(S(C)(=O)=O)ccc1Nc1nc(N[C@H]2CC[C@](C)(O)CC2)c2cccn2n1 ; CCOc1cc(-c2nncn2C)ccc1Nc1nc(NC2CCCCC2)c2c(C#N)ccn2n1]

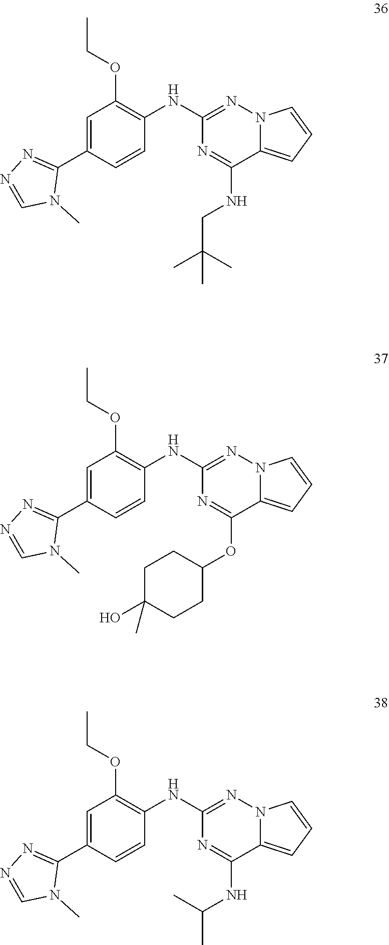 [SMILES: CCOc1cc(-c2nncn2C)ccc1Nc1nc(NCC(C)(C)C)c2cccn2n1 ; CCOc1cc(-c2nncn2C)ccc1Nc1nc(OC2CCC(C)(O)CC2)c2cccn2n1 ; CCOc1cc(-c2nncn2C)ccc1Nc1nc(NC(C)C)c2cccn2n1]

In some other embodiments, the present disclosure relates to a pharmaceutical composition, which includes an effective amount of the compound as described above.

In some other embodiments, the pharmaceutical composition further includes a pharmaceutically acceptable carrier, adjuvant, vehicle or a combination thereof.

In some other embodiments, the pharmaceutical composition further includes one or more therapeutic agents selected from other anti-tumor drugs.

In some other embodiments, the therapeutic agent is an antimitotic agent, an alkylating agent, an antimetabolic drug, a topoisomerase inhibitor, an estrogen receptor modulator, an androgen receptor modulator, a protein kinase targeting small molecule inhibitor, and a protein kinase targeting antibody drug.

In some other embodiments, the antimitotic agent is paclitaxel or vincristine.

In some other embodiments, the alkylating agent is cisplatin, oxaliplatin, carboplatin or cyclophosphamide.

In some other embodiments, the antimetabolite is gemcitabine, 5-fluorouracil or methotrexate.

In some other embodiments, the topoisomerase inhibitor is epipodophyllotoxin, etoposide, topotecan or camptothecin.

In some other embodiments, the estrogen receptor modulator is tamoxifen or ulvestrant.

In some other embodiments, the androgen receptor modulator is bicalutamide.

In some other embodiments, the protein kinase targeting small molecule inhibitor is dasatinib, bosutinib, gefitinib, erlotinib, rapatinib, imatinib, nilotinib, sorafenib, tipifanib, sunitinib, and acitinib.

In some other embodiments, the protein kinase targeting antibody drug is trastuzumab, panizumab and cetuximab.

In some other embodiments, the present disclosure relates to use of the compound or the pharmaceutical composition in the preparation of drugs for preventing, ameliorating, treating or alleviating diseases related to TTK overexpression or hyperactivity in patients.

In some other embodiments, the TTK overexpression related disease is tumors.

In some other embodiments, the tumors are papillary thyroid carcinoma, breast cancer, gastric cancer, bronchial cancer or lung cancer.

In some other embodiments, the present disclosure relates to use of the compound or the pharmaceutical composition in the preparation of drugs for inhibiting TTK.

Unless otherwise specified, the present disclosure includes all stereoisomers, geometric isomers, tautomers, solvates, hydrates, metabolites, salts and pharmaceutically acceptable prodrugs of the compound of the present disclosure.

In some embodiments, the salt refers to a pharmaceutically acceptable salt. The term “pharmaceutically acceptable” means that a substance or composition need to be chemically and/or toxicologically compatible with the other ingredients constituting the preparation and/or the mammals treated with it.

The compound of the present disclosure further includes a salt form, which is not necessarily a pharmaceutically acceptable salt, but from which the compound of the present disclosure can be prepared or purified and/or an intermediate from which an enantiomer of the compound of the present disclosure can be separated.

The compound of the present disclosure, including its salts, can also be obtained in the form of a hydrate, or contain other solvents for crystallization. The compound of the present disclosure can inherently form or form a solvate with a pharmaceutically acceptable solvent (include water) by design. Therefore, the present disclosure also includes a solvated and unsolvated form.

Moreover, the compound of the present disclosure may contain several asymmetric centers or a commonly described racemic mixture. The present disclosure further includes a racemic mixture, a partial racemic mixture, and an enantiomers and a diastereomer obtained by separation.

The compound of the present disclosure can exist in the form of one of a possible isomer, a rotamer, an atropisomer and a tautomer or in the form of a mixture thereof. The present disclosure can further include a mixture of an isomer, a rotamer, an atropisomer, and a tautomer of the compound of the present disclosure, a partial mixture of an isomer, a rotamer, an atropisomer, and a tautomer, or separated isomer, rotamer, atropisomer, or tautomer.

Moreover, the compound mentioned in the present disclosure includes a compound defined in the present disclosure that is labeled with various isotopes, for example, compounds labeled with radioactive isotopes such as ³H, ¹⁴C and ¹⁸F, or compounds labeled with non-radioactive isotopes such as ²H and ¹³C.

In another aspect, the present disclosure relates to a method for preparing, separating and purifying the compound of Formula (I).

The foregoing only outlines some aspects of the invention, and the present disclosure is not limited thereto. These and other aspects will be described in more detail below.

### DEFINITIONS AND GENERAL TERMS

Some embodiments of the present disclosure will be described in detail, examples of which are illustrated by the attached structural formulas and chemical formulas. The present disclosure is intended to cover all alternatives, modifications and equivalents, which are all embraced within the scope of the present disclosure as defined by the claims. Those skilled in the art will realize that many methods and materials similar or equivalent to those described in the present disclosure can be used to practice this invention. The present disclosure is not limited to the methods and materials described in the present disclosure in any way. In case that one or more of the incorporated documents, patents and similar materials are inconsistent with or contradict this application (including, but not limited to, defined terms, terminologies, described technologies, and others), this application is shall prevail.

It should be further recognized that some features of the present disclosure described in several independent embodiments for the sake of clarity can also be provided in combination in a single embodiment. On the contrary, various features of the present disclosure described in a single embodiment for the sake of brevity can also be provided separately or in any suitable sub-combinations.

Unless otherwise indicated, the technical and scientific terms used in the present disclosure have the same meaning as commonly understood by those skilled in the art to which this invention belongs. Unless otherwise indicated, all patent publications cited in throughout the disclosure of the present disclosure are incorporated herein by reference in their entireties.

The following definitions are applicable to the present disclosure, unless otherwise indicated. For the purpose of the present disclosure, chemical elements are defined according to the periodic table, CAS version, and Handbook of Chemicals, 75^{th} Ed, 1994. In addition, the general principles of organic chemistry can be found in “Organic Chemistry”, Thomas Sorrell, University Science Books, Sausalito: 1999, and “March's Advanced Organic Chemistry”, by Michael B. Smith and Jerry March, John Wiley & Sons, New York: 2007. Therefore, references are incorporated throughout the disclosure of the present disclosure.

The term “subject” used in the present disclosure refers to animals, Typically the animal is a mammal. The subject may also be primates (such as humans), cattle, sheep, goats, horses, dogs, cats, rabbits, rats, mice, fish, birds and so on. In some embodiments, the subject is a primate. In yet other embodiments, the subject is a human.

The terms “subject” and “patient” used in the present disclosure are interchangeable. The terms “subject” and “patient” refer to animals (for example, birds such as chickens, quails or turkeys, or mammals), especially “mammals” including non-primates (such as cattle, pigs, horses, sheep, rabbits, guinea pigs, rats, cats, dogs and mice) and primates (such as monkeys, chimpanzees and humans). In one embodiment, the subject is a non-human animal, such as a domestic animal (e.g., horse, cow, pig or sheep) or a pet (e.g., dog, cat, guinea pig or rabbit). In other embodiments, “patient” refers to human beings.

The present disclosure also includes an isotopically labeled compound of the present disclosure, which is the same as those described in the present disclosure except for the fact that one or more atoms are replaced by atoms with atomic mass or mass number different from that of common atoms found in nature. Exemplary isotopes that can also be introduced into the compound of the present disclosure include isotopes of hydrogen, carbon, nitrogen, oxygen, phosphorus, sulfur, fluorine and chlorine, such as ²H, ³H, ¹³C, ¹⁴C, ¹⁵N, ¹⁶O, ¹⁷O, ³¹P, ³²P, ³⁶S, ¹⁸F and ³⁷Cl.

The compound of the present disclosure containing the aforementioned isotopes and/or other isotopes of other atoms and a pharmaceutically acceptable salt of the compound are covered in the scope of the present disclosure. In case of isotopically labeled compound of the present disclosure, for example, the introduction of a radioactive isotope, such as ³H and ¹⁴C into the compound of the present disclosure is useful for tissue distribution analysis of drugs and/or substrates. Because of the ease of preparation and detection, isotopes such as tritium (i.e. ³H) and carbon-14 (i.e. ¹⁴C) are particularly preferred. In addition, the substitution with a heavier isotope, such as deuterium, that is, ²H, can provide some therapeutic advantages due to greater metabolic stability, such as increased in vivo half-life or reduced dosage requirement. Therefore, it is preferable in some cases.

Stereochemical definitions and conventions used in the present disclosure are generally in accordance with S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compound of the present disclosure may have an asymmetric center or chiral center, and thus exist in different stereoisomeric forms. It is expected that all stereoisomeric forms of the compound of the present disclosure, including, but not limited to, a diastereomer, an enantiomer, an atropisomer, and a mixture thereof such as a racemic mixture, are also included in the scope of the present disclosure. Many organic compounds exist in the optically active forms, that is, they have the ability to rotate the plane of plane polarized light. When an optically active compound is described, the prefixes d and L or R and S are used to represent the absolute configuration of a molecule at a chiral center (or multiple chiral centers) in the molecule. The prefixes d and l or (+) and (−) are symbols used to indicate the rotation of plane polarized light caused by the compound, in which (−) or 1 indicates that the compound is left-handed, and the prefixes (+) or d indicates that the compound is right-handed. As far as a given chemical structure is concerned, these stereoisomers are the same except that they are mirror images of each other. Specific stereoisomers can also be called enantiomers, and mixtures of these isomers are usually called mixtures of enantiomers. A 50:50 mixture of enantiomers is called racemic mixture or raceme, which can be present when there is no stereoselectivity or stereospecificity in a chemical reaction or method.

Depending on the choices of raw materials and methods, the compound of the present disclosure can exist in the form of one or a mixture of possible isomers. For example, it exists as a pure optical isomer, or as an isomeric mixture, for example, an racemic and diastereomeric mixture, depending on the number of asymmetric carbon atoms. The optically active (R)- or (S)-isomer can be prepared by chiral synthesis or chiral preparation, or resolved by conventional techniques. If the compound contains a double bond, the substituent may be in the E or Z configuration; and if the compound contains a disubstituted cycloalkyl group, the substituents on the cycloalkyl group may be in the cis- or trans-configuration.

The compound of the present disclosure may have an asymmetric center or chiral center, and thus exist in different stereoisomeric forms. It is expected that all stereoisomeric forms of the compound of the present disclosure, including, but not limited to, a diastereomer, an enantiomer, an atropisomer, a geometric (conformational) isomer and a mixture thereof such as a racemic mixture, are also included in the scope of the present disclosure.

Unless otherwise specified, the structure described in the present disclosure also refers to all isomeric forms including this structure (e.g., enantiomer, diastereomer, atropisomer and geometric (or conformational) isomer), for example, R and S configurations of various asymmetric centers, (Z) and (E) double bond isomers, and (Z) and (E) conformational isomers. Therefore, the single stereochemical isomers, enantiomeric mixture, diastereomeric mixture and geometric isomer (or conformational isomer) mixture of the compound of the present disclosure are all embraced in the scope of the present disclosure.

The term “tautomer” or “tautomeric form” refers to structural isomers with different energies that can be converted into each other by overcoming a low energy barrier. If tautomerism is possible (such as in a solution), the chemical equilibrium of tautomers can be achieved. For example, a protontautomer (also called prototropic tautomer) includes interconversion through proton migration, such as ketone-enol isomerization and imine-enamine isomerization. Valence tautomers include interconversion through the rearrangement of some bonded electrons. The specific example of ketone-enol tautomerism is the tautomerism of pentan-2,4-dione and 4-hydroxypent-3-en-2-one tautomers. Another example of tautomerism is phenol-ketone tautomerism. A specific example of phenol-ketone tautomerism is the tautomerism of pyridin-4-ol and pyridin-4(1H)-one tautomers. Unless otherwise indicated, all tautomeric forms of the compound of the present disclosure are contemplated in the scope of the present disclosure.

The “N-oxide” used in the present disclosure means that when the compound contains several amine functional groups, one or more nitrogen atoms can be oxidized to form an N-oxide. Special examples of N-oxide are N-oxide of a tertiary amine or N-oxide of a nitrogen containing heterocyclic ring. The corresponding amine can be treated with an oxidant such as hydrogen peroxide or peracid (e.g. peroxycarboxylic acid) to form an N-oxide (see Advanced Organic Chemistry, Wiley Interscience, 4th Edition, Jerry March, pages). In particular, an N-oxide can be prepared by the L. W. Deady method (Syn. Comm. 1977, 7, 509-514), in which, for example, an amine compound is reacted with m-chloroperbenzoic acid (MCPBA) in an inert solvent such as dichloromethane.

The solvate in the present disclosure refers to the compound of the present disclosure in association with one or more solvent molecules. Solvents that form solvates include, but are not limited to, water, isopropanol, ethanol, methanol, dimethyl sulfoxide, ethyl acetate, acetic acid and aminoethanol. The term “hydrate” means an association compound formed with water as the solvent molecule.

“Metabolite” refers to a product of a specific compound or its salt formed through metabolism in vivo. A metabolite of a compound can be identified by techniques well known in the art, and the activity can be characterized by experimental methods as described in the present disclosure. Such a product can be obtained through the oxidation, reduction, hydrolysis, amidation, deamidation, esterification, deesterification, enzymatic lysis and so on of the administered compound. Accordingly, the invention includes a metabolite of the compound, including a metabolite produced by fully contacting the compound of the present disclosure with a mammal for a period of time.

“Pharmaceutically acceptable salt” as used in the present disclosure refer to an organic salt and an inorganic salt of the compound of the present disclosure. Pharmaceutically acceptable salts are well known to is in the art, for example, those described in S. M. Berge et al., describe pharmaceutically acceptable salts in detail in J. Pharmaceutical Sciences, 1977, 66: 1-19. Pharmaceutically acceptable non-toxic salts formed with acids include, but are not limited to, salts formed by reacting an amino group with inorganic acid, such as hydrochloride, hydrobromide, phosphate, sulfate, and perchlorate; and salts with organic acids, such as acetate, oxalate, maleate, tartrate, citrate, succinate, malonate, or salts obtained by other methods such as ion exchange recorded in books and documents. Other pharmaceutically acceptable salts include adipate, alginate, ascorbate, aspartate, benzenesulfonate, benzoate, bisulfate, borate, butyrate, camphorate, camphorsulfonate, cyclopentylpropionate, digluconate, dodecylsulfate, ethanesulfonate, formate, fumarate, glucoheptonate, glycerophosphate, gluconate, hemisulfate, heptanate, caproate, hydroiodide, 2-hydroxy-ethanesulfonate, galacturonate, lactate, laurate, laurylsulfate, malate, malonate, methanesulfonate, 2-naphthalensulfonate, nicotinate, nitrate, oleate, palmitate, pamoate, pectate, persulfate, 3-phenylpropionate, picrate, pivalate, propionate, stearate, thiocyanate, p-toluenesulfonate, undecanate, valerate, and so on. Salts formed by reacting with suitable bases include salts of alkali metal, alkaline earth metal, ammonium and N⁺(C₁₋₄ alkyl)₄. The present disclosure also contemplates any quatemary ammonium salt formed by a compound containing a N group. Water-soluble, oil-soluble or dispersible products can be obtained by quaternization. Alkali metal or alkaline earth metal salts include sodium, lithium, potassium, calcium, magnesium, and so on. The pharmaceutically acceptable salts further include appropriate, nontoxic amine cations formed by ammonium and quaternary ammonium salts with counterions, such as halides, hydroxides, carboxylates, sulfates, phosphates, nitrates, C₁₋₈ sulfonates and aromatic sulfonates.

The term “prodrug” used in the present disclosure refers to a compound that is converted into a compound of Formula (I) in vivo. Such conversion is affected by the hydrolysis of the prodrug in blood or the enzymatic conversion in blood or tissues into the parent structure. The prodrug compound of the present disclosure can be an ester. In related art, esters useful as a prodrug includes phenyl esters, aliphatic (C₁₋₂₄) esters, acyloxymethyl esters, carbonates, aminocarboxylate esters and amino acid esters. For example, one compound of the present disclosure contains a hydroxyl group, then it can be acylated to obtain a compound as a prodrug. Other prodrug forms include phosphates, for example, obtained by phosphorylation of a hydroxyl group on the parent compounds. For a complete discussion of prodrugs, reference can be made to T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, J. Rautio et al., Prodrugs. Design and Clinical Applications, Nature Review Drug Discovery, 2008, 7, 255-270, and S. J. Hecker et al., Prodrugs of Phosphates and Phosphonates, Journal of Medicinal Chemistry, 2008, 51, 2328-2345.

Any asymmetric atom (for example, carbon) in the compound of the present disclosure can exist in the form of racemically or enantiomerically enriched configuration, for example (R)-, (S)- or (R, S)-configuration. In some embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess with respect to the (R)- or (S)-configuration. If possible, a substituent on an atom with an unsaturated double bond may be in the cis-(Z)- or trans-(E)-form.

Therefore, as described in the present disclosure, the compound of the present disclosure can exist in the form of one of possible isomers, rotamer, atropisomer, tautomer, or in the form of a mixture thereof, for example, substantially pure geometric (cis or trans) isomer, diastereomer, optical isomer (enantiomer), raceme or a mixture thereof.

Any isomeric mixture obtained can be separated into pure or substantially pure geometric or optical isomers, diastereomers or racemes according to the physical and chemical differences of the components, for example, by chromatography and/or fractional crystallization.

The racemate of any final product or Intermediate obtained can be resolved into optical enantiomers by a known method familiar to those skilled in the art, for example, by separating a diastereomeric salt obtained. The racemic products can also be separated by chiral chromatography, such as high pressure liquid chromatography (HPLC) using chiral adsorbents. Particularly, an enantiomer can be prepared by asymmetric synthesis (for example, Jacques, et al., Enantiomers, Racemates and Resolutions (Wiley Interscience, New York, 1981); Principles of Asymmetric Synthesis (2^{nd} Ed. Robert E. Gawley, Jeffrey Aube, Elsevier, Oxford, U K, 2012); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, Ind. 1972).

As described in the present disclosure, the compound of the present disclosure can be optionally substituted with one or more substituents, as does the compound represented a general formula above, or special examples in the examples, and subclasses and a class of compounds included in the present disclosure. It should be understood that the term “optionally substituted” and the term “substituted or unsubstituted” can be used interchangeably. The terms “optionally” or “optional” means that the event or condition described later may but does not necessarily occur, and the description includes the situation in which the event or condition occurs and the situation in which the event or condition does not occur. Generally, the term “optionally”, whether preceding the term “substituted” or not, means that one or more hydrogen atoms in a given structure are replaced by specific substituents. Unless otherwise indicated, the substitution with an optional substituent can occur at various substitutable positions of the group. When more than one position in a given structural formula can be substituted with one or more substituents selected from specific groups, the substitution with the substituents can occur at the same or different positions. The substituent can be, but is not limited to, F, Cl, Br, CN, N₃, OH, NH₂, NO₂, oxo (═O), —C(═O)R^{a}, —C(═O)OR^{b}, —S(═O)₂R^{b}, —C(═O)NR^{c}R^{d}, OR^{b}, —NR^{c}R^{d}, R^{b}O—C₁₋₄ alkylene, R^{d}R^{c}N—C₁₋₄ alkylene, C₁₋₆ alkyl, C₁₋₆ aliphatic groups, C₁₋₆ haloalkyl, C₁₋₆ alkoxy, C₁₋₆ alkylamino, C₂₋₆ alkenyl, C₂₋₆ alkynyl, C₃₋₈ cycloalkyl, C₃₋₈ cycloalkyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 14 atoms or (heteroaryl having 5 to 14 atoms)-C₁₋₄ alkylene, in which R^{a}, R^{b}, R^{c}, and R^{d} have the meanings as defined herein.

In addition, it is to be understood that unless otherwise explicitly pointed out, the descriptions “each . . . is independent”, “ . . . are each independently” and “ . . . is independently” used in the present disclosure are interchangeable, and should be understood in a broad sense. They mean that specific options expressed by the same symbols in different groups do not affect each other, or that specific options expressed by the same symbols in the same group do not affect each other.

In various sections of the specification, the substituents of the compound disclosed in the present disclosure are disclosed according to the group type or range. In particular, the present disclosure includes each independent sub-combination of various members in the type or group of groups. For example, the term “C₁₋₆ alkyl” particularly indicates methyl, ethyl, C₃ alkyl, C₄ alkyl, C₅ alkyl and C₆ alkyl disclosed individually.

In various sections of the specification, linking substituents are described. When a structure clearly requires a linking group, the Markush variable given for the group should be understood as a linking group. For example, if a structure requires a linking group, and the Markush group definition for this variable gives “alkyl” or “aryl”, it should be understood that the “alkyl” or “aryl” respectively represents an alkylene or arylene linking group.

The term “alkyl” or “alkyl group” used in the present disclosure means a saturated straight or branched monovalent hydrocarbyl radical containing 1-20 carbon atoms. Unless otherwise specified in detail, the alkyl group contains 1-20 carbon atoms. In some embodiments, the alkyl group contains 1-10 carbon atoms; in some other embodiments, the alkyl group contains 1-9 carbon atoms; in some other embodiments, the alkyl group contains 1-8 carbon atoms; in some other embodiments, the alkyl group contains 1-6 carbon atoms; in some other embodiments, the alkyl group contains 1-4 carbon atoms; and in some other embodiments, the alkyl group contains 1-3 carbon atoms.

Examples of alkyl groups include, but are not limited to, methyl (Me, —CH₃), ethyl (Et, —CH₂CH₃), n-propyl (n-Pr, —CH₂CH₂CH₃), isopropyl (i-Pr, —CH(CH₃)₂), n-butyl (n-Bu, —CH₂CH₂CH₂CH₃), isobutyl (i-Bu, —CH₂CH(CH₃)₂), s-butyl (s-Bu, —CH(CH₃)CH₂CH₃), t-butyl (t-Bu, —C(CH₃)₃), n-pentyl (—CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), n-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), n-heptyl, n-octyl, and others, in which the alkyl group can be independently unsubstituted or substituted with one or more substituents described herein.

The term “alkyl group” and its prefix “alkyl” used in the present disclosure both contain a straight or branched saturated carbon chain.

The term “alkylene” refers to a saturated divalent hydrocarbyl group obtained by removing two hydrogen atoms from a linear or branched saturated hydrocarbyl group. Unless otherwise specified in detail, the alkylene group contains 1-10 carbon atoms. In some other embodiments, the alkylene group contains 1-6 carbon atoms; in some other embodiments, the alkyl group contains 1-4 carbon atoms; and in some other embodiments, the alkyl group contains 1-2 carbon atoms. Examples include methylene (—CH₂—), ethylene (—CH₂CH₂—), isopropylene (—CH(CH₃)CH₂—) and so on, where the alkylene group may be independently unsubstituted or substituted with one or more substituents described in the present disclosure.

The term “alkenyl” refers to a straight or branched monovalent hydrocarbyl group with 2-12 carbon atoms, 2-8 carbon atoms, 2-6 carbon atoms, or 2-4 carbon atoms, in which the C—C at least one position is in an unsaturated state of sp² double bond. The alkenyl group can be independently unsubstituted or substituted with one or more substituents described in the present disclosure and includes group with “cis”, “trans” or “Z” “E” configurations. Specific examples include, but are not limited to, ethenyl (—CH═CH₂), allyl (—CH₂CH═CH₂), and so on.

The term “alkynyl” refers to a straight or branched monovalent hydrocarbyl group with 2-12 carbon atoms, 2-8 carbon atoms, 2-6 carbon atoms, or 2-4 carbon atoms, in which the C—C at least one position is in an unsaturated state of sp triple bond. The alkynyl group can be independently unsubstituted or substituted with one or more substituents described in the present disclosure. Specific examples include, but are not limited to, ethynyl (—C≡CH), propargyl (—CH₂C≡CH), 1-propynyl (—C≡C—CH₃), and the like.

The term “alkoxy” means an alkyl group that is attached to the rest of a molecule via an oxygen atom, where the alkyl group has the meaning as defined in the present disclosure. Unless otherwise specified in detail, the alkoxy group contains 1-20 carbon atoms. In some embodiments, the alkoxy group contains 1-10 carbon atoms; in some other embodiments, the alkoxy group contains 1-8 carbon atoms; in some other embodiments, the alkoxy group contains 1-6 carbon atoms; in some other embodiments, the alkoxy group contains 1-4 carbon atoms; and in some other embodiments, the alkoxy group contains 1-3 carbon atoms.

Examples of alkoxy groups include, but are not limited to, methoxy (MeO, —OCH₃), ethoxy (EtO, —OCH₂CH₃), 1-propoxy (n-PrO, n-propoxy, —OCH₂CH₂CH₃), 2-propoxy (i-PrO, i-propoxy, —OCH(CH₃)₂), 1-butoxy (n-BuO, n-butoxy, —OCH₂CH₂CH₂CH₃), 2-methyl-1-propoxy (i-BuO, i-butoxy, —OCH₂CH(CH₃)₂), 2-butoxy (s-BuO, s-butoxy, —OCH(CH₃)CH₂CH₃), 2-methyl-2-propoxy (t-BuO, t-butoxy, —OC(CH₃)₃), 1-pentyloxy (n-pentyloxy, —OCH₂CH₂CH₂CH₂CH₃), 2-pentyloxy (—OCH(CH₃)CH₂CH₂CH₃), 3-pentyloxy (—OCH(CH₂CH₃)₂), 2-methyl-2-butoxy (—OC(CH₃)₂CH₂CH₃), 3-methyl-2-butoxy (—OCH(CH₃)CH(CH₃)₂), 3-methyl-1-butoxy (—OCH₂CH₂CH(CH₃)₂), 2-methyl-1-butoxy (—OCH₂CH(CH₃)CH₂CH₃), and the like. The alkoxy group can be independently unsubstituted or substituted with one or more substituents described in the present disclosure.

The term “haloalkyl”, “haloalkenyl” or “haloalkoxy” refers to an alkyl, alkenyl or alkoxy group substituted with one or more halogen atoms. Examples include, but are not limited to, trifluoromethyl, and trifluoromethoxy, etc.

The term “carbocyclic ring”, “carbocyclyl” or “carbocyclic” are used interchangeably here, and refers to a non-aromatic carbocyclic system that is saturated or includes one or more unsaturated units and has 3-14 ring carbon atoms. In some embodiments, the number of carbon atoms is 3-12; in other embodiments, the number of carbon atoms is 3-10; in other embodiments, the number of carbon atoms is 3-8; in other embodiments, the number of carbon atoms is 5-6; and in other embodiments, the number of carbon atoms is 6-8. The “carbocyclyl” includes monocyclic, bicyclic or polycyclic fused, spiro or bridged carbocyclic ring systems, and also a polycyclic ring system in which a carbocyclic ring can be fused to one or more nonaromatic carbocyclic rings or heterocyclic rings or one or more aromatic rings or a combination thereof, in which the linking group or point is on the carbocyclic ring. The bicyclic carbocyclyl group includes bridged bicyclic carbocyclyl, fused bicyclic carbocyclyl and spirobicyclic carbocyclyl. The “fused” bicyclic ring system contains two rings sharing two adjacent ring atoms. The bridged bicyclic group includes two rings sharing 3 or 4 adjacent ring atoms. The spiral ring system shares 1 ring atom. Suitable carbocyclyl groups include, but are not limited to, cycloalkyl, cycloalkenyl and cycloalkynyl. Examples of carbocyclyl groups further include, but are not limited to, cyclopropyl, cyclobutyl, cyclopentyl, 1-cyclopentyl-1-alkenyl, 1-cyclopentyl-2-alkenyl, 1-cyclopentyl-3-alkenyl, cyclohexyl, 1-cyclohexyl-1-alkenyl, 1-cyclohexyl-2-alkenyl, 1-cyclohexyl-3-alkenyl, cyclohexadienyl, cycloheptyl, cyclooctyl, cyclononyl, cyclodecyl, cycloundecyl, and cyclododecanyl, etc. Bridged carbocyclyl groups include, but are not limited to, bicyclo[2.2.2]octyl, bicyclo[2.2.1] heptyl, bicyclo[3.3.1]nonyl, bicyclo[3.2.3]nonyl, and so on.

The term “cycloalkyl” refers to a saturated monocyclic, bicyclic or tricyclic system with 3-12 ring carbon atoms, having one or more points of connection to the rest of a molecule. In some embodiments, the cycloalkyl is a ring system containing 3-10 ring carbon atoms; in some other embodiments, the cycloalkyl is a ring system containing 3-8 ring carbon atoms; in some other embodiments, the cycloalkyl is a ring system containing 3-6 ring carbon atoms; and in some other embodiments, the cycloalkyl is a ring system containing 5-6 ring carbon atoms. The cycloalkyl group can be independently unsubstituted or substituted with one or more substituents described in the present disclosure.

The terms “heterocyclyl group” and “heterocyclic ring” are used interchangeably here, and refer to a saturated or partially unsaturated, nonaromatic monocyclic, bicyclic or tricyclic system containing 3-12 ring atoms, in which at least one ring atom is selected from nitrogen, sulfur and oxygen, and this ring system has one or more points of connection to the rest of the molecule. The term “heterocyclyl” includes monocyclic, bicyclic or polycyclic fused, spiro or bridged heterocyclic ring systems, and also a polycyclic ring system in which a heterocyclic ring can be fused to one or more nonaromatic carbocyclic rings or heterocyclic rings or one or more aromatic rings or a combination thereof, in which the linking group or point is on the heterocyclic ring. The bicyclic heterocyclyl group includes bridged bicyclic heterocyclyl groups, fused bicyclic heterocyclyl groups and spirobicyclic heterocyclyl groups. Unless otherwise specified, the heterocyclyl groups can be a C-group or a N-group, and the —CH₂— group can be optionally replaced by —C(═O)—. The sulfur atom on the ring can optionally be oxidized into S-oxide. The nitrogen atom on the ring can optionally be oxidized into N-oxide. In some embodiments, the heterocyclyl is a monocyclic or bicyclic heterocyclic group having 3-8 atoms; in some other embodiments, the heterocyclyl is a monocyclic or bicyclic heterocyclic group having 3-6 atoms; in some other embodiments, the heterocyclyl is a monocyclic or bicyclic heterocyclic group having 6-8 atoms; in some other embodiments, the heterocyclyl is a heterocyclic group having 5-6 atoms; in some other embodiments, the heterocyclyl is a heterocyclic group having is 4 atoms; in some other embodiments, the heterocyclyl is a heterocyclic group having 5 atoms; in some other embodiments, the heterocyclyl is a heterocyclic group having 6 atoms; in some other embodiments, the heterocyclyl is a heterocyclic group having 7 atoms; and in some other embodiments, the heterocyclyl is a heterocyclic group having 8 atoms.

Examples of heterocyclyl groups include, but are not limited to, oxiranyl, azetidinyl, oxetanyl, thietanyl, pyrrolidinyl, 2-pyrrolinyl, 3-pyrrolinyl, pyrazolinyl, pyrazolidinyl, imidazolinyl, imidazolidinyl, tetrahydrofuryl, dihydrofuryl, tetrahydrothienyl, dihydrothienyl, 1,3-dioxolanyl, dithiolanyl, tetrahydropyranyl, dihydropyranyl, 2H-pyranyl, 4H-pyranyl, tetrahydrothiopyranyl, piperidinyl, morpholinyl, thiomorpholinyl, piperazinyl, dioxanyl, dithianyl, thioxanyl, homopiperazinyl, homopiperidinyl, oxepanyl, thiepanyl, oxazepinyl, diazepinyl, thiazepinyl, indolinyl, 1,2,3,4-tetrahydroisoquinolyl, 1,3-benzodioxolyl, and 2-oxa-5-azabicylco[2.2.1]heptan-5-yl. Examples of heterocylcyl groups in which —CH₂— is replaced by —C(═O)— include, but are not limited to, 2-oxopyrrolidinyl, oxo-1,3-thiazolidinyl, 2-piperidinonyl, 3,5-dioxopiperidyl, and pyrimidinedionyl. Examples of heterocylcyl groups in which sulfur is oxidized include, but are not limited to, sulfolanyl and 1,1-dioxothiomorpholinyl. The bridged heterocyclyl groups include, but are not limited to, 2-oxabicylco[2.2.2]octyl, 1-azabicylco[2.2.2]octyl, 3-azabicylco[3.2.1]octyl, and the like. The heterocyclyl group can be optionally substituted with one or more substituents described in the present disclosure.

The term “bridge” refers to a bond, atom or unbranched atomic chain connecting two different moieties of a molecule. Two atoms (usually but not always two tertiary carbon atoms) connected by a bridge are “bridgeheads”.

The term “spiro” refers to a ring system with an atom (usually quaternary carbon atom) that is the only common atom between two rings.

The term “having n atoms”, where n is an integer, typically describes the number of ring-forming atoms in a molecule, where the ring-forming atoms in the molecule is n. For example, the piperidinyl is a heterocyclic group having 6 atoms, and 1,2,3,4-tetrahydronaphthyl is a carbocyclyl group having 10 atoms.

The term “heteroatom” refers to O, S, N, P and Si, including any oxidized form of N, S and P; primary, secondary tertiary amine, and quaternary ammonium salt forms; or a form in which hydrogen on the nitrogen atom in the heterocyclic ring is substituted, for example, N (like N in 3, 4-dihydro-2H-pyrrolyl), NH (like NH in pyrrolidinyl) or NR (like NR in N-substituted pyrrolidinyl).

The term “halogen” refers to F, Cl, Br or I.

The term “N₃” represents an azide structure. This group can be linked to other groups, for example, it can be linked to a methyl group to form azidomethane (MeN₃), or to a phenyl group to form azidobenzene (PhN₃).

The term “aryl” can be used alone or as a large moiety of “aralkyl”, “aralkoxy” or “aryloxyalkyl”, and indicates monocyclic, bicyclic and tricyclic carbocyclic systems containing 6-14 ring atoms, or 6-12 ring atoms, or 6-10 ring atoms, in which at least one ring system is aromatic, and each ring system contains a ring having 3-7 atoms, and has one or more points of attachment to the rest of the molecule. The term “aryl” can be used interchangeably with the term “aromatic ring” or “aryl ring”. For example, the aromatic ring can include phenyl, naphthyl and anthracyl. The aryl group may be independently unsubstituted or substituted with one or more substituents described in the present disclosure.

The term “heteroaryl” can be used alone or as a large moiety of “heteroarylalkyl” or “heteroarylalkoxy”, and indicates a monocyclic, bicyclic and tricyclic systems containing 5-14 ring atoms, 5-12 ring atoms, 5-10 ring atoms, or 5-6 ring atoms, in which at least one ring system is aromatic, and at least one ring system contains one or more heteroatoms; and each ring system contains a ring having 5-7 atoms and has one or more points of attachment to the rest of the molecule. Unless otherwise specified, the aryl group can be a C-group or a N-group, and the —CH₂— group can be optionally replaced by —C(═O)—. The sulfur atom on the ring can optionally be oxidized into S-oxide. The nitrogen atom on the ring can optionally be oxidized into N-oxide. The term heteroaryl can be used interchangeably with the term “heteroaromatic ring” or “heteroaromatic compound”. In some embodiments, the heteroaryl is a heteroaryl group having 5-12 atoms and containing 1, 2, 3 or 4 heteroatoms independently selected from O, S and N. In some other embodiments, the heteroaryl is a heteroaryl group having 5-10 atoms and containing 1, 2, 3 or 4 heteroatoms independently selected from O, S and N. In some other embodiments, the heteroaryl is a heteroaryl group having 5-6 atoms and containing 1, 2, 3 or 4 heteroatoms independently selected from O, S and N. In some embodiments, the heteroaryl is a heteroaryl group having 5 atoms and containing 1, 2, 3 or 4 heteroatoms independently selected from O, S and N. In some other embodiments, the heteroaryl is a heteroaryl group having 6 atoms and containing 1, 2, 3 or 4 heteroatoms independently selected from O, S and N.

In some other embodiments, the heteroaryl includes, but is not limited to the following monocyclic groups: 2-furyl, 3-furyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, pyridazinyl (for example, 3-pyridazinyl), 2-thiazolyl, 4-thiazolyl, 5-thiazolyl, tetrazolyl (for example, 5H-tetrazolyl, 2H-tetrazolyl), triazolyl (for example, 2-triazolyl, 5-triazolyl, 4H-1,2,4-triazolyl, 1H-1,2,4-triazolyl, 1,2,3-triazolyl), 2-thienyl, 3-thienyl, pyrazolyl (for example, 2-pyrazolyl and 3-pyrazolyl), isothiazolyl, 1,2,3-oxadiazolyl, 1,2,5-oxadiazolyl, 1,2,4-oxadiazolyl, 1,3,4-oxadiazolyl, 1,2,3-thiodiazolyl, 1,3,4-thiodiazolyl, 1,2,5-thiodiazolyl, pyrazinyl, and 1,3,5-triazinyl; and includes, but is not limited to the following dicyclic groups: benzoimidazolyl, benzofuryl, benzothienyl, indolyl (for example, 2-indolyl), purinyl, quinolyl (for example, 2-quinolyl, 3-quinolyl, 4-quinolyl), isoquinolyl (for example, 1-isoquinolyl, 3-isoquinolyl or 4-isoquinolyl), oxathiayl,

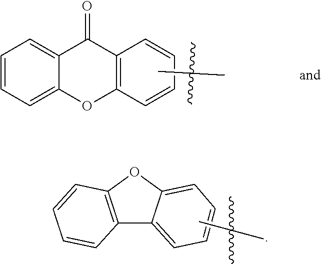

The heteroaryl group is optionally substituted with one or more substituents described in the present disclosure.

The term “carboxyl”, whether used alone or in combination with other terms, for example, “carboxyalkyl”, represents —CO₂H. The term “carbonyl”, whether used alone or in combination with other terms, such as “aminocarbonyl” or “acyloxy”, means —(C═O)—.

The term “alkylamino” includes “N-alkylamino” and “N,N-dialkylamino”, in which the amino groups are respectively independently replaced by one or two alkyl groups. In some embodiments, the alkylamino is a lower alkylamino group with one or two C₁₋₆ alkyl groups attached to the nitrogen atom. In some other embodiments, the alkylamino is a lower C₁₋₃alkylamino group. Suitable alkylamino groups can be mono- or dialkylamino groups. Examples include, but are not limited to, N-methylamino, N-ethylamino, N,N-dimethylamino, N,N-diethylamino, and the like.

The term “arylamino” means an amino group that is replaced by one or two aryl groups. Examples include, but are not limited to, N-phenylamino. In some embodiments, the aryl ring on the arylamino can be further substituted.

The term “aminoalkyl” includes a linear or branched C₁₋₁₀ alkyl group substituted with one or more amino groups. In some embodiments, the aminoalkyl is a lower C₁₋₆“aminoalkyl” substituted with one or more amino groups. Examples include, butyl are not limited to, aminomethyl, aminoethyl, aminopropyl, aminobutyl and aminohexyl.

As described in the present disclosure, a ring system formed by linking a substituent through a bond drawn to a center of a ring means that the substitution with the substituent can occur at any substitutable positions on the ring. This ring system includes a mono-, di- or polycyclic system.

The term “unsaturated” used in the present disclosure means that the group contains one or more unsaturations.

The term “comprises” or “includes” is an open expression, that is, it includes the contents indicated in the present disclosure, but does not exclude other contents.

As described in present disclosure, the term “pharmaceutically acceptable carrier” includes any solvent, dispersion medium, coating material, surfactant, antioxidant, preservative (such as antibacterial agent and antifungal agent), isotonic agent, salt, drug stabilizer, binder, excipient, dispersant, lubricant, sweetener, flavoring agent, colorant, or a combination thereof. These carriers are known to those skilled in the art (as described in Remington's Pharmaceutical Sciences, 18th Ed. Mack Printing Company, 1990, pp. 1289-1329). Except for any situation where a conventional carrier is incompatible with the active ingredient, the use thereof in a therapeutic or pharmaceutical composition is covered.

As used in the present disclosure, the term “inhibiting TTK” includes reducing the expression or activity of TTK (for example, by at least 10%) and completely inhibiting the expression or activity of TTK (that is, inhibiting the expression or activity of TTK by 100%). In some embodiments, the expression or activity of TTK is inhibited by at least 50%, at least 65%, at least 75%, at least 85%, at least 90% or at least 95%.

The term “effective amount” of the compound of the present disclosure refers to an amount that causes an expected biological response. In the present disclosure, the expected biological response is to inhibit TTK, prevent the recurrence, development, attack or progress of symptoms associated with TTK overexpression, or enhance or improve the preventive or therapeutic effect of another anti-tumor therapy used. The exact amount of the compound to be administered to a subject will depend on the mode of administration, severity, and status of the subject, such as health status, age, sex, weight and drug tolerance. An appropriate dosage can be determined by the technicians based on these and other factors. When administered in combination with other anti-tumor agents, such as antimitotic agent, the “effective amount” of the second agent will depend on the type of the drug used. An appropriate dosage of an approved agent is known and can be adjusted by technicians according to the subject's condition, the type of condition treated and the amount of the compound of the present disclosure used. If the quantity is not specified clearly, an effective quantity should be adopted. For example, the compound of the present disclosure can be administered to a subject at a dosage in the range of about 0.01-100 mg/body weight/day for therapeutic or preventive treatment.

The term “treatment” used in the present disclosure refers to therapeutic and preventive treatment. For example, the therapeutic treatment includes alleviating or improving the progress, severity and/or duration of TTK overexpression- or overactivity-mediated disorders, or improving one or more symptoms (particularly, one or more recognizable symptoms) of TTK overexpression- or overactivity-mediated disorders, by administering one or more therapies (for example, one or more therapeutic agents (for example the compound and composition of the present disclosure)). In a specific embodiment, the therapeutic treatment includes improving at least one measurable physical parameter of TTK overexpression- or overactivity-mediated disorders. In other embodiments, the therapeutic treatment includes inhibiting the progress of TTK overexpression- or overactivity-mediated disorders, for example, by stabilizing recognizable symptoms physically and/or or stabilizing physical parameters physiologically. In other embodiments, the therapeutic treatment includes alleviating or stabilizing TTK overexpression- or overactivity-mediated disorders, such as papillary thyroid carcinoma, breast cancer, gastric cancer, bronchial cancer or lung cancer.

The term “protecting group” or “PG” refers to a substituent that usually serves to block or protect special functionality when reacts with other functional groups. For example, “an amino protecting group” means a substituent attached to an amino group that serves to block or protect the functionality of the amino group in a compound. Suitable amino protecting groups include acetyl, trifluoroacetyl, tert-butoxycarbonyl (BOC, Boc), benzyloxycarbonyl (CBZ, Cbz) and 9-fluorenylmethoxycarbonyl (Fmoc). Similarly, “a hydroxyl protecting group” refers to a substituent of a hydroxyl group that serves to block or protect the functionality of the hydroxyl group. Suitable protecting groups include acetyl and silyl. A “carboxyl protecting group” refers to a substituent of a carboxyl group that serves to block or protect the functionality of the carboxyl group. General carboxyl protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrobenzenesulfonyl)ethyl, 2-(diphenylphosphino)ethyl, nitroethyl, and the like. For a general description of the protecting groups, reference can be made to T W. Greene, Protective Groups in Organic Synthesis, John Wiley & Sons, New York, 1991; and P. J. Kocienski, Protecting Groups, Thieme, Stuttgart, 2005.

### DESCRIPTION OF COMPOUND OF THE PRESENT DISCLOSURE

The present disclosure provides a pyrrolo[2,1-f][1,2,4]triazine derivative, which shows potent inhibitory activity on TTK, and has the potential to be used as a novel TTK inhibitor in the treatment of tumors.

The present disclosure provides a compound, which is a compound of Formula (I) or a stereoisomer, a tautomer, a N-oxide, a solvate, a metabolite, a pharmaceutically acceptable salt or a prodrug of the compound of Formula (I):

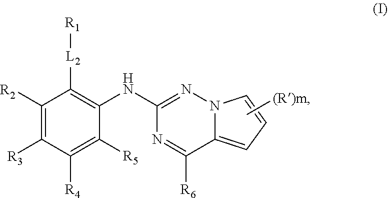

where R₁, R₂, R₃, R₄, R₅, R₆, L₂, R′ and m have the meanings as defined herein.

In some other embodiments, L₂ is a bond or 0;

R₁, R₂, R₄, and R₅ are each independently H, F, Cl, Br, CN, NO₂, —OR^{b}, —NR^{c}R^{d}, or C₁₋₆ alkyl;

R₃ is —C(═O)R^{a}, —C(═O)OR^{b}, —S(═O)₂R^{b}, —C(═O)NR^{c}R^{d}, —OR^{b}, —NR^{c}R^{d}, R^{b}O—C₁₋₄ alkylene, R^{d}R^{c}N—C₁₋₄ alkylene, C₁₋₆ alkyl, C₂₋₆ alkenyl, C₂₋₆ alkynyl, C₃₋₈ cycloalkyl, C₃₋₈ cycloalkyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 10 atoms, or (heteroaryl having 5 to 10 atoms)-C₁₋₄ alkylene, in which the C₁₋₆ alkyl, C₂₋₆ alkenyl, C₂₋₆ alkynyl, C₃₋₈ cycloalkyl, C₃₋₈ cycloalkyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 10 atoms and (heteroaryl having 5 to 10 atoms)-C₁₋₄ alkylene are each independently unsubstituted or substituted with 1, 2, 3 or 4 substituents independently selected from F, Cl, Br, CN, ═O, —OR^{b}, —NR^{C}R^{d}, C₁₋₆ alkyl, C₁₋₆ haloalkyl, R^{b}O—C₁₋₄ alkylene, or R^{d}R^{c}N—C₁₋₄ alkylene;

R₆ is H or

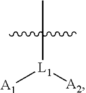

in which L₁ is N or O;

A₁ and A₂ are each independently H, C₁₋₆ alkyl, C₃₋₁₂ carbocyclyl, C₃₋₁₂ carbocyclyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 14 atoms, or (heteroaryl having 5 to 14 atoms)-C₁₋₄ alkylene, or A₁ and A₂, together with L₁ to which they are attached, form a heterocyclic ring having 3-6 atoms, in which the C₁₋₆ alkyl, C₃₋₁₂ carbocyclyl, C₃₋₁₂ carbocyclyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 14 atoms, (heteroaryl having 5 to 14 atoms)-C₁₋₄ alkylene, or heterocyclic ring having 3-6 atoms formed by A₁ and A₂ together with L₁ to which they are attached are each independently unsubstituted or substituted with 1, 2, 3, 4 or 5 R′, provided that A₁ and A₂ are not both H;

each R′ is independently H, F, Cl, Br, CN, NO₂, ═O, —OR^{b}, —NR^{c}R^{d}, —S(═O)₂R^{b}, C₁₋₆ alkyl|, C₁₋₆ haloalkyl, C₃₋₈ cycloalkyl, C₃₋₈ cycloalkenyl, heterocyclyl having 3 to 12 atoms, C₆₋₁₀ aryl or heteroaryl having 5 to 10 atoms, in which the C₃₋₈ cycloalkyl, heterocyclyl having 3 to 12 atoms, C₆₋₁₀ aryl or heteroaryl having 5 to 10 atoms are each independently unsubstituted or substituted with 1, 2, 3 or 4 substituents independently selected from F, Cl, Br, CN, ═O, —OR^{b}, —NR^{C}R^{d}, C₁₋₆ alkyl, C₁₋₆ haloalkyl, R^{b}O—C₁₋₄ alkylene or R^{d}R^{c}N—C₁₋₄ alkylene;

m is 0, 1, 2 or 3;

provided that when R₆ is H, m is not 0, and at least one R′ is C₃₋₈ cycloalkyl, C₃₋₈ cycloalkenyl, heterocyclyl having 3 to 12 atoms, C₆₋₁₀ aryl or heteroaryl having 5 to 10 atoms, in which the C₃₋₈ cycloalkyl, C₃₋₈ cycloalkenyl, heterocyclyl having 3 to 12 atoms, C₆₋₁₀ aryl heteroaryl having 5 to 10 atoms are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene; and

R^{a}, R^{b}, R^{c}, and R^{d} are each independently H, C₁₋₆ alkyl, C₁₋₆ haloalkyl, or heterocyclyl having 3 to 6 atoms, or R^{c} and R^{d}, together with nitrogen to which they are attached, form a heterocyclic ring having 3 to 6 atoms, in which the C₁₋₆ alkyl and heterocyclic ring having 3 to 6 atoms are each independently unsubstituted or substituted with 1, 2, 3 or 4 substituents independently selected from F, Cl, CN, OH, NH₂, C₁₋₆ alkyl, C₁₋₆ haloalkyl, C₁₋₆ alkoxy or C₁₋₆ alkylamino.

In some other embodiments, the present disclosure relates to a compound of Formula (II) or a stereoisomer, a tautomer, a N-oxide, a solvate, a metabolite, a pharmaceutically acceptable salt or a prodrug of the compound of Formula (II):

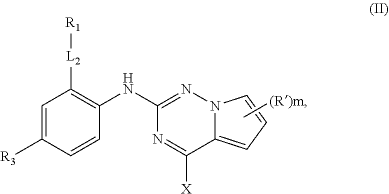

where R₁, R₃, L₂, R′, X and m have the meanings as defined herein.

In some other embodiments, X is represented by a sub-structural formula below:

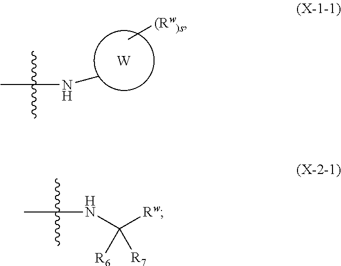

where ring W is C₃₋₈ cycloalkyl, a heterocyclic ring having 3 to 8 atoms, benzene or a heteroaryl ring having 5 to 6 atoms; each R^{w} is independently H, F, Cl, Br, CN, NO₂, ═O, —OR^{b}, —NR^{c}R^{d}, —S(═O)₂R^{b}, C₁₋₆ alkyl or C₁₋₆ haloalkyl; R₇ and R₈ are each independently H, or C₁₋₆ alkyl; and s is 0, 1, 2 or 3.

In some other embodiments, the present disclosure relates to a compound of Formula (III) or a stereoisomer, a tautomer, a N-oxide, a solvate, a metabolite, a pharmaceutically acceptable salt or a prodrug of the compound of Formula (III):

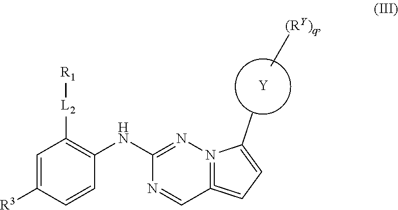

where R₁, R₃, L₂, ring Y, R^{Y} and q have the meanings as defined herein.

In some other embodiments, ring Y is C₃₋₈ cycloalkyl, C₃₋₈ cycloalkenyl, a heterocyclic ring having 3 to 8 atoms, benzene or a heteroaryl ring having 5 to 6 atoms; each R^{Y} is independently H, F, Cl, Br, CN, NO₂, ═O, —OR^{b}, —NR^{c}R^{d}, —S(═O)₂R^{b}, C₁₋₆ alkyl or C₁₋₆ haloalkyl; and q is 0, 1, 2 or 3.

In some other embodiments, L₁ is N.

In some other embodiments, L₁ is O.

In some other embodiments, L₂ is a bond.

In some other embodiments, L₂ is O.

In some other embodiments, R₂, R₄, and R₅ are each independently H.

In some other embodiments, R₃ is —C(═O)NR^{c}R^{d}, OR^{b}, —NR^{c}R^{d}, —S(═O)₂R^{b}, heterocyclyl having 3 to 6 atoms, (heterocyclyl having 3 to 6 atoms)-C₁₋₄ alkylene, C₆₋₉ aryl, C₆₋₉ aryl-C₁₋₄ alkylene, heteroaryl having 5 to 9 atoms or (heteroaryl having 5 to 9 atoms)-C₁₋₄ alkylene.

In some other embodiments, R₆ is H, upon which m is not 0, and at least one R′ is C₃₋₈ cycloalkyl, C₃₋₈ cycloalkenyl, heterocyclyl having 3 to 12 atoms, C₆₋₁₀ aryl or heteroaryl having 5 to 10 atoms, in which the C₃₋₈ cycloalkyl, C₃₋₈ cycloalkenyl, heterocyclyl having 3 to 12 atoms, C₆₋₁₀ aryl or heteroaryl having 5 to 10 atoms are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene.

In some other embodiments, R₆ is

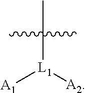

In some other embodiments, A₁ and A₂ are each independently H, C₁₋₆ alkyl, C₃₋₆ carbocyclyl, C₃₋₆ carbocyclyl-C₁₋₄ alkylene, heterocyclyl having 3 to 6 atoms, (heterocyclyl having 3 to 6 atoms)-C₁₋₄ alkylene, C₆₋₈ aryl, C₆₋₈ aryl-C₁₋₄ alkylene, heteroaryl having 5 to 8 atoms, or (heteroaryl having 5 to 8 atoms)-C₁₋₄ alkylene, or A₁ and A₂, together with L₁ to which they are attached, form a heterocyclic ring having 3 to 6 atoms.

In some other embodiments, A₁ and A₂ are each independently H, or C₁₋₆ alkyl, provided that A₁ and A₂ are not H.

In some other embodiments, A₁ and A₂ are each independently H, C₃₋₆ carbocyclyl, C₃₋₆ carbocyclyl-C₁₋₄ alkylene, heterocyclyl having 3 to 6 atoms, (heterocyclyl having 3 to 6 atoms)-C₁₋₄ alkylene, C₆₋₈ aryl, C₆₋₈ aryl-C₁₋₄ alkylene, heteroaryl having 5 to 8 atoms, or (heteroaryl having 5 to 8 atoms)-C₁₋₄ alkylene, provided that A₁ and A₂ are not both H.

In some other embodiments, A₁ and A₂, together with L₁ to which they are attached, form a heterocyclic ring having 3 to 6 atoms.

In some other embodiments, each R′ is independently H, F, Cl, Br, CN, NO₂, ═O, —OR_{b}, —NR_{c}R_{d}, —S(═O)OR^{b}, C₁₋₃ alkyl, C₁₋₃ haloalkyl, C₃₋₆ cycloalkyl, heterocyclyl having 3 to 6 atoms, C₆₋₈ aryl or heteroaryl having 5 to 8 atoms, in which the C₃₋₆ cycloalkyl, heterocyclyl having 3 to 6 atoms, C₆₋₈ aryl or heteroaryl having 5 to 8 atoms are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene.

In some other embodiments, each R′ is independently H.

In some other embodiments, each R′ is independently F, Cl, or Br.

In some other embodiments, each R′ is independently CN.

In some other embodiments, R₆ is

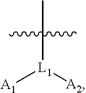

and m is 0, 1, 2 or 3.

In some other embodiments, R^{a}, R^{b}, R′, and R^{d} are each independently H, methyl, ethyl, isopropyl, n-propyl, n-butyl, t-butyl, C₁₋₃ haloalkyl, or heterocyclyl having 3 to 6 atoms, or R^{c} and R^{d}, together with nitrogen to which they are attached, form a heterocyclic ring having 3 to 6 atoms.

In some other embodiments, A₁ and A₂ are each independently H, C₁₋₆ alkyl, C₃₋₆ carbocyclyl, heterocyclyl having 3 to 6 atoms, C₆₋₈ aryl, or heteroaryl having 5 to 8 atoms.

In some other embodiments, R₃ is —C(═O)NR^{c}R^{d}, OR^{b}, —NR^{c}R^{d}, —S(═O)₂R^{b}, heterocyclyl having 3 to 6 atoms, phenyl, naphthyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, pyrimidinyl, indolyl, purinyl, quinolyl, isoquinolyl, or phenoxathiinyl, in which the heterocyclyl having 3 to 6 atoms, phenyl, naphthyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, pyrimidinyl, indolyl, purinyl, quinolyl, isoquinolyl, or phenoxathiinyl are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene.

In some other embodiments, R₃ is —C(═O)NR^{c}R^{d}.

In some other embodiments, R₃ is OR^{b}.

In some other embodiments, R₃ is —NR^{c}R^{d}.

In some other embodiments, R₃ is —S(═O)₂R^{b}.

In some other embodiments, R₃ is heterocyclyl having 3 to 6 atoms.

In some other embodiments, R₃ is phenyl.

In some other embodiments, R₃ is naphthyl.

In some other embodiments, R₃ is pyrrolyl.

In some other embodiments, R₃ is pyridinyl.

In some other embodiments, R₃ is pyrazolyl.

In some other embodiments, R₃ is imidazolyl.

In some other embodiments, R₃ is triazolyl.

In some other embodiments, R₃ is tetrazolyl.

In some other embodiments, R₃ is oxazolyl.

In some other embodiments, R₃ is oxadiazolyl.

In some other embodiments, R₃ is 1,3,5-triazinyl.

In some other embodiments, R₃ is thiazolyl.

In some other embodiments, R₃ is thienyl.

In some other embodiments, R₃ is pyrazinyl.

In some other embodiments, R₃ is pyridazinyl.

In some other embodiments, R₃ is pyrimidinyl.

In some other embodiments, R₃ is indolyl.

In some other embodiments, R₃ is purinyl.

In some other embodiments, R₃ is quinolyl.

In some other embodiments, R₃ is isoquinolyl.

In some other embodiments, R₃ is phenoxathiinyl.

In some other embodiments, ring W is C₃₋₆ cycloalkyl, heterocyclyl having 3 to 6 atoms, phenyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, or pyrimidinyl.

In some other embodiments, ring W is C₃₋₆ cycloalkyl.

In some other embodiments, ring W is heterocyclyl having 3 to 6 atoms.

In some other embodiments, ring W is phenyl.

In some other embodiments, ring W is C₃₋₆ cycloalkyl, heterocyclyl having 3 to 6 atoms, phenyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, or pyrimidinyl.

In some other embodiments, ring W is heterocyclyl having 3 to 6 atoms.

In some other embodiments, ring W is C₃₋₆ cycloalkyl.

In some other embodiments, ring W is phenyl.

In some other embodiments, ring W is pyrrolyl.

In some other embodiments, ring W is pyridinyl.

In some other embodiments, ring W is pyrazolyl.

In some other embodiments, ring W is imidazolyl.

In some other embodiments, ring W is triazolyl.

In some other embodiments, ring W is tetrazolyl.

In some other embodiments, ring W is oxazolyl.

In some other embodiments, ring W is oxazolyl.

In some other embodiments, ring W is oxadiazolyl.

In some other embodiments, ring W is 1,3,5-triazinyl.

In some other embodiments, ring W is thiazolyl.

In some other embodiments, ring W is thienyl.

In some other embodiments, ring W is pyrazinyl.

In some other embodiments, ring W is thienyl.

In some other embodiments, ring W is pyrazinyl.

In some other embodiments, ring W is pyridazinyl.

In some other embodiments, ring W is pyrimidinyl.

In some other embodiments, R^{w} is independently H, F, Cl, Br, CN, NO₂, ═O, —OR^{b}, —NR^{c}R^{d}, —S(═O)₂R^{b}, methyl, ethyl, isopropyl, n-propyl, n-butyl or t-butyl or C₁₋₆ haloalkyl.

In some other embodiments, ring Y is C₃₋₆ cycloalkyl, C₃₋₆ cycloalkenyl, heterocyclyl having 3 to 6 atoms, phenyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, pyrimidinyl, 3,6-dihydro-2H-pyran or tetrahydro-2H-pyran.

In some other embodiments, ring Y is C₃₋₆ cycloalkyl.

In some other embodiments, ring Y is C₃₋₆ cycloalkenyl.

In some other embodiments, ring Y is heterocyclyl having 3 to 6 atoms.

In some other embodiments, ring Y is phenyl.

In some other embodiments, ring Y is pyrrolyl.

In some other embodiments, ring Y is pyridinyl.

In some other embodiments, ring Y is pyrazolyl.

In some other embodiments, ring Y is imidazolyl.

In some other embodiments, ring Y is triazolyl.

In some other embodiments, ring Y is tetrazolyl.

In some other embodiments, ring Y is oxazolyl.

In some other embodiments, ring Y is oxadiazolyl.

In some other embodiments, ring Y is 1,3,5-triazinyl.

In some other embodiments, ring Y is thiazolyl.

In some other embodiments, ring Y is thienyl.

In some other embodiments, ring Y is pyrazinyl.

In some other embodiments, ring Y is pyridazinyl.

In some other embodiments, ring Y is pyrimidinyl.

In some other embodiments, ring Y is 3,6-dihydro-2H-pyran or tetrahydro-2H-pyran.

In some other embodiments, R^{Y} is independently H, F, Cl, Br, CN, NO₂, ═O, —OR^{b}, —NR^{c}R^{d}, —S(═O)₂R^{b}, methyl, ethyl, isopropyl, n-propyl, n-butyl or t-butyl or C₁₋₆ haloalkyl.

In some other embodiments, the present disclosure relates to a compound of Formula (IV) or a stereoisomer, a tautomer, a N-oxide, a solvate, a metabolite, a pharmaceutically acceptable salt or a prodrug of the compound of Formula (IV):

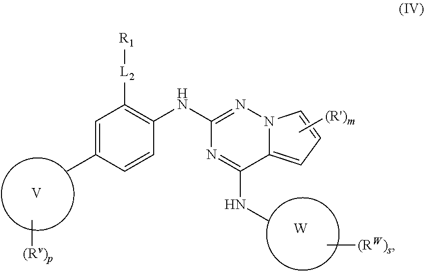

where ring V, ring W, R₁, L₂, R′, R^{w}, R^{V}, s, p, and m have the meanings as defined herein.

In some other embodiments, the present disclosure relates to a compound of Formula (V) or a stereoisomer, a tautomer, a N-oxide, a solvate, a metabolite, a pharmaceutically acceptable salt or a prodrug of the compound of Formula (V):

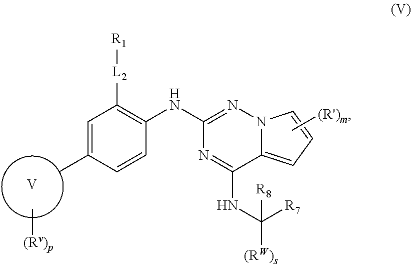

where ring V, R₁, L₂, R′, R^{w}, R^{V}, s, p, and m have the meanings as defined herein, and R₇ and R₈ are each independently H, or C₁₋₆ alkyl.

In some other embodiments, the present disclosure relates to a compound of Formula (VI) or a stereoisomer, a tautomer, a N-oxide, a solvate, a metabolite, a pharmaceutically acceptable salt or a prodrug of the compound of Formula (VI):

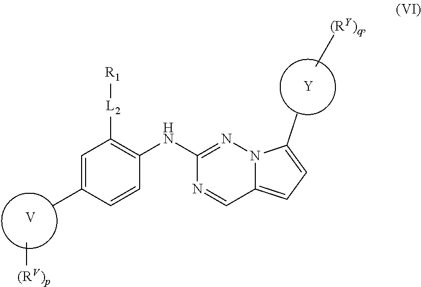

where ring V, ring Y, R₁, L₂, R^{V}, R^{V}, p, and q have the meanings as defined herein.

In some other embodiments, the present disclosure relates to a compound having a structure of Formula (VII) or a stereoisomer, a tautomer, a N-oxide, a solvate, a metabolite, a pharmaceutically acceptable salt or a prodrug of the compound of Formula (VII):

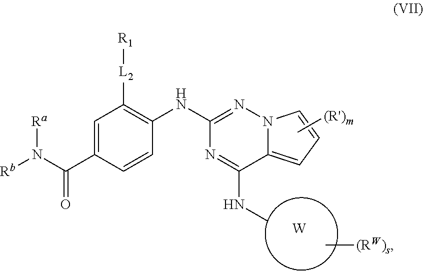

where ring W, R₁, R^{a}, R^{b}, L₂, R′, R^{w}, s, and m have the meanings as defined herein.

In some other embodiments, the present disclosure relates to a compound of Formula (VIII) or a stereoisomer, a tautomer, a N-oxide, a solvate, a metabolite, a pharmaceutically acceptable salt or a prodrug of the compound of Formula (VIII):

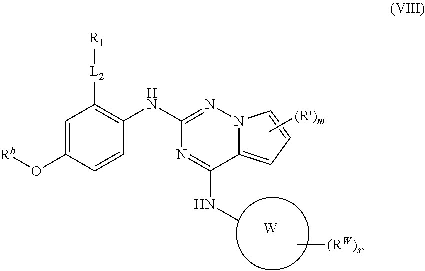

where ring W, R₁, R^{b}, L₂, R′, R^{w}, s, and m have the meanings as defined herein.

In some other embodiments, the present disclosure relates to a compound having a structure of Formula (VIIII) or a stereoisomer, a tautomer, a N-oxide, a solvate, a metabolite, a pharmaceutically acceptable salt or a prodrug of the compound of Formula (VIIII):

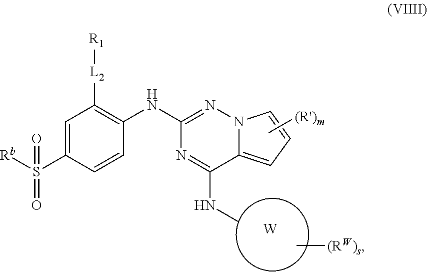

where ring W, R₁, R^{b}, L₂, R′, R^{w}, s, and m have the meanings as defined herein.

In some other embodiments, ring V is C₃₋₈ cycloalkyl, heterocyclic ring having 3 to 8 atoms, benzene or heteroaryl ring having 5 to 6 atoms; R^{V} is F, Cl, Br, CN, —OH, ═O, C₁₋₆ alkyl or C₁₋₆ haloalkyl; p is 0, 1, 2 or 3.

In some other embodiments, ring V is C₃₋₈ cycloalkyl

In some other embodiments, ring V is phenyl.

In some other embodiments, ring V is naphthyl.

In some other embodiments, ring V is pyrrolyl.

In some other embodiments, ring V is pyridinyl.

In some other embodiments, ring V is pyrazolyl.

In some other embodiments, ring V is imidazolyl.

In some other embodiments, ring V is triazolyl.

In some other embodiments, ring V is tetrazolyl.

In some other embodiments, ring V is oxazolyl.

In some other embodiments, ring V is oxadiazolyl.

In some other embodiments, ring V is 1,3,5-triazinyl.

In some other embodiments, ring V is thiazolyl.

In some other embodiments, ring V is thienyl.

In some other embodiments, ring V is pyrazinyl.

In some other embodiments, ring V is pyridazinyl.

In some other embodiments, ring V is pyrimidinyl.

In some other embodiments, ring V is indolyl.

In some other embodiments, ring V is purinyl.

In some other embodiments, ring V is quinolyl.

In some other embodiments, ring V is isoquinolyl.

In some other embodiments, ring V is phenoxathiinyl.

In an aspect, the present disclosure provides a pharmaceutical composition including the compound of the present disclosure.

In some embodiments of the present disclosure, the pharmaceutical composition further includes a pharmaceutically acceptable carrier, adjuvant, vehicle or a combination thereof.

In some embodiments, the pharmaceutical composition provided in the present disclosure further includes one or more therapeutic agents.

In some embodiments, the therapeutic agent is an antimitotic agent, an alkylating agent, an antimetabolic drug, a topoisomerase inhibitor, an estrogen receptor modulator, an androgen receptor modulator, a protein kinase targeting small molecule inhibitor, and a protein kinase targeting antibody drug.

In some embodiments, the antimitotic agent is paclitaxel or vincristine.

In some embodiments, the alkylating agent is cisplatin, oxaliplatin, carboplatin or cyclophosphamide.

In some embodiments, the topoisomerase inhibitor is epipodophyllotoxin, etoposide, topotecan or camptothecin.

In some embodiments, the estrogen receptor modulator is tamoxifen or ulvestrant.

In some embodiments, the androgen receptor modulator is bicalutamide.

In some embodiments, the protein kinase targeting small molecule inhibitor is dasatinib, bosutinib, gefitinib, erlotinib, rapatinib, imatinib, nilotinib, sorafenib, tipifanib, sunitinib, and acitinib.

In some embodiments, the protein kinase targeting antibody drug is trastuzumab, panizumab and cetuximab.

In some other embodiments, the pharmaceutical composition can be in the dosage form of a liquid, a solid, a semi-solid, a gel or a spray.

In some other embodiments, the present disclosure relates to use of the compound or the pharmaceutical composition in the preparation of drugs for preventing, ameliorating, treating or alleviating diseases related to TTK overexpression or hyperactivity in patients.

In some other embodiments, the TTK overexpression related disease is tumors.

In some other embodiments, the tumors are papillary thyroid carcinoma, breast cancer, gastric cancer, bronchial cancer or lung cancer.

In some other embodiments, the present disclosure relates to use of the compound or the pharmaceutical composition in the preparation of drugs for inhibiting TTK.

In some embodiments, the salt refers to a pharmaceutically acceptable salt. The term “pharmaceutically acceptable” means that a substance or composition need to be chemically and/or toxicologically compatible with the other ingredients constituting the preparation and/or the mammals treated with it.

The compound of the present disclosure further includes such an additional salt of the compound, which is not necessarily a pharmaceutically acceptable salt, but from which the compound of the present disclosure can be prepared or purified and/or an intermediate from which an enantiomer of the compound of the present disclosure can be separated.

Pharmaceutically acceptable acid addition salts can be formed with inorganic and organic acids, such as acetate, aspartate, benzoate, benzenesulfonate, bromide/hydrobromide, bicarbonate/carbonate, bisulfate/sulfate, camphorsulfonate, chloride/hydrochloride, chlorotheophylline salt, citrate, ethanedisulfonate, fumarate, glucoheptonate, glyconate, glucuronate, hippurate, hydroiodide/iodide, hydroxyethylsulfonate, lactate, galacturonate, laurylsulfate, malate, maleate, malonate, mandelate, methanesulfonate, methylsulfate, naphthalenecarboxylate, naphthalenesulfonate, nicotinate, nitrate, octadecanoate, oleate, oxalate, palmitate, pamoate, phosphate/hydrophosphate/dihydric phosphate, polygalacturonate, propionate, stearate, succinate, sulfosalicylate, tartrate, tosylate and trifluoroacetate.

Inorganic acids from which salts can be derived include, for example, hydrochloric acid, hydrobromic acid, sulfuric acid, nitric acid, phosphoric acid and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, p-toluenesulfonic acid and sulfosalicylic acid.

Pharmaceutically acceptable base addition salts can be formed with inorganic bases or organic bases.

Inorganic bases from which salts can be derived include, for example, ammonium salts and metals from Groups I to XII of the Periodic Table. In some embodiments, the salt is derived from sodium, potassium, ammonium, calcium, magnesium, iron, silver, zinc and copper. Particularly suitable salts include ammonium, potassium, sodium, calcium and magnesium salts.

Organic bases from which salts can be derived include primary amines, secondary amines, tertiary amines, and substituted amines including naturally occurring substituted amines, cyclic amines and basic ion exchange resins. Some organic amines include, for example, isopropylamine, benzathine, cholinate, diethanolamine, diethylamine, lysine, meglumine, piperazine and tromethamine.

The pharmaceutically acceptable salts of the present disclosure can be synthesized from the parent compound, alkaline or acidic moiety by conventional chemical methods. Generally, such salts can be prepared by reacting these compounds in free acid forms with stoichiometric amounts of appropriate bases (such as Na, Ca, Mg or K hydroxide, carbonate, and bicarbonate, etc.), or by reacting these compounds in free base forms with stoichiometric amounts of appropriate acids. Such reactions are usually carried out in water or an organic solvent or a mixture of them. Generally, in an appropriate circumstance, a non-aqueous medium needs to be used, such as ether, ethyl acetate, ethanol, isopropanol or acetonitrile. List of some other suitable salts can be found in “Remington's Pharmaceutical Sciences”, 20th edition, Mack Publishing Company, Easton, Pa., (1985); and “Handbook of Pharmaceutical Salts: Properties, Selection, and Use”, Stahl and Wermuth (Wiley-VCH, Weinheim, Germany, 2002).

Moreover, the compound of the present disclosure, including its salts, can also be obtained in the form of a hydrate, or contain other solvents for crystallization. The compound of the present disclosure can inherently form or form a solvate with a pharmaceutically acceptable solvent (include water) by design. Therefore, the present disclosure is intended to includes a solvated and unsolvated form.

Any structural formula given in the present disclosure is also intended to represent unlabeled forms and isotopically labeled forms of these compounds. The isotopically labeled compound has the structure represented by the general formula given in the present disclosure, except that one or more atoms are replaced by an atom with the selected atomic weight or mass number. Exemplary isotopes that can be introduced into the compound of the present disclosure include isotopes of hydrogen, carbon, nitrogen, oxygen, phosphorus, sulfur, fluorine and chlorine, such as ²H, ³H, ¹¹C, ¹³C, ¹⁴C, ¹⁵N, ¹⁸F, ³¹P, ³²P, ³⁶S, ³⁷Cl or ¹²⁵I.

Moreover, the compound mentioned in the present disclosure includes a compound defined in the present disclosure that is labeled with various isotopes, for example, compounds labeled with radioactive isotopes such as ³H, ¹⁴C and ¹⁸F, or compounds labeled with non-radioactive isotopes such as ²H and ¹³C. Such isotopically labeled compounds can be used in metabolic studies (¹⁴C), reaction kinetics studies (for example, ²H or ³H), detection or imaging techniques, such as positron emission tomography (PET) or single photon emission computed tomography (SPECT) including the determination of tissue distribution of drug or substrate, or radiotherapy of patients. ¹⁸F-labeled compounds are particularly desirable for PET or SPECT research. The isotopically labeled compound of Formula (I) can be prepared by conventional techniques familiar to those skilled in the art or as described in the examples and preparation process of the present disclosure, using an appropriate isotopically labeled reagent in place of the previously used unlabeled reagent.

In addition, the substitution with a heavier isotope, especially deuterium (that is, 2H or D) can provide some therapeutic advantages brought about by the higher metabolic stability, for example, increased in-vivo half-life, lowered dosage demand or improved therapeutic index. It should be understood that deuterium in this context is regarded as a substituent of the compound of Formula (I). The concentration of such heavier isotopes, especially deuterium, can be defined by isotope enrichment factor. The term “isotope enrichment factor” used in the present disclosure refers to the ratio of the isotopic abundance and natural abundance of a specified isotope. If the substituent of the compound of the present disclosure is designated as deuterium, the compound has an isotope enrichment factor of at least 3500 (with 52.5% deuterium doped at each designated position of deuterium atom), at least 4000 (with 60% deuterium doped), at least 4500 (with 67.5% deuterium doped), at least 5000 (with 75% deuterium doped), at least 5500 (with 82.5% deuterium doped), at least 6000 (with 90% deuterium doped), at least 6333.3 (with 95% deuterium doped), at least 6466.7 (with 97% deuterium doped), at least 6600 (with 99% deuterium doped) or at least 6633.3 (with 99.5% deuterium doped) for each indicated deuterium atom. Pharmaceutically acceptable solvates of the present disclosure include those in which the crystallization solvent can be isotopically substituted, such as D₂O, acetone-d₆, or DMSO-d₆.

Composition, Preparation and Administration of the Compound of the Present Disclosure

The present disclosure provides a pharmaceutical composition, which includes an effective amount of the compound of the present disclosure or a stereoisomer thereof. According to a specific embodiment of the present disclosure, the pharmaceutical composition further includes at least a pharmaceutically acceptable carrier, diluent, adjuvant or vehicle, and optionally, other therapeutic and/or preventive ingredients. In some embodiments, the pharmaceutical composition includes an effective amount of at least a pharmaceutically acceptable carrier, diluent, adjuvant or vehicle.

The pharmaceutically acceptable carrier may contain an inert component that do not excessively inhibit the biological activity of the compound. The pharmaceutically acceptable carrier needs to be biocompatible, for example, it is non-toxic, non-inflammatory, non-immunogenic or causes no other adverse reactions or side effects when administered to a patient. A standard pharmaceutical technology can be used.

As described in the present disclosure, the pharmaceutical composition or pharmaceutically acceptable composition of the present disclosure further includes a pharmaceutically acceptable carrier, adjuvant or excipient, including any solvent, diluent, liquid excipient, dispersant, suspending agent, surfactant, isotonic agent, thickener, emulsifier, preservative, solid binder or lubricant, and the like, which are suitable for the specific target dosage form as sued in the present disclosure. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers for use in the preparation of pharmaceutically acceptable compositions and known preparation methods therefor. Except for conventional carrier media that are incompatible with the compound of the present disclosure, due to for example adverse biological effects or harmful interactions with any other components in the pharmaceutically acceptable composition, any other conventional carrier media and their uses are also contemplated in the present disclosure.

Some examples of substances that can be used as pharmaceutically acceptable carriers include, but are not limited to, ion exchangers, alumina, aluminum stearate, lecithin, serum proteins (such as human serum albumin), buffer substances (such as Tween 80, phosphate, glycine, sorbic acid or potassium sorbate), partial glyceride mixtures of saturated vegetable fatty acids, water, salts or electrolytes (such as protamine sulfate, disodium hydrogen phosphate, potassium hydrogen phosphate, sodium chloride or zinc salt), silica gel, magnesium trisilicate, polyvinylpyrrolidone, polyacrylate, wax, polyethylene-polypropylene oxide block copolymer, methyl cellulose, hydroxypropyl methyl cellulose, lanolin, sugars (such as lactose, glucose and sucrose), starch (such as corn starch and potato starch), cellulose and its derivatives (such as sodium carboxymethylcellulose, ethyl cellulose and cellulose acetate), powdered tragacanth, malt, gels, talc, excipients (such as cocoa butter and suppository wax), oils (such as peanut oil, cottonseed oil, safflower oil, sesame oil, olive oil, corn oil and soybean oil), ethylene glycol (such as propylene glycol or polyethylene glycol), esters (such as ethyl oleate and ethyl dodecanoate), agar, buffers (such as magnesium hydroxide and aluminum hydroxide), alginic acid, pyrogen free water, isotonic saline, Ringer's solution, ethanol, phosphate buffer, other non-toxic compatible lubricants (such as sodium lauryl sulfate and magnesium stearate), and colorants, anti-sticking agents, coating agents, sweeteners, flavoring agents, preservers and antioxidants that can also be present in the composition according to the judgment of the preparation personnel.

The compound or composition of the present disclosure can be administered through any suitable route. The above-mentioned compound and pharmaceutically acceptable composition can be administered to humans or other animals orally, rectally, parenterally, intracisternally, vaginally, intraperitoneally, topically (for example, by powder, ointment or drops), or buccally as a mouth or nasal spray, etc. according to the severity of the disease to be treated.

The liquid dosage form for oral administration includes, but is not limited to, pharmaceutically acceptable emulsions, microemulsions, solutions, suspensions, syrups and elixirs. In addition to the active compound, the liquid dosage form may further contain a commonly used inert diluent in the art, for example, water or other solvents, solubilizers and emulsifiers, such as ethanol, isopropanol, ethyl carbonate, ethyl acetate, benzyl alcohol, benzyl benzoate, propylene glycol, 1,3-butanediol, dimethyl formamide, oils (particularly cottonseed oil, peanut oil, corn oil, germ oil, olive oil, castor oil and sesame oil), glycerin, tetrahydrofurfuryl alcohol, polyethylene glycol and fatty acid sorbitan ester or a mixture thereof. In addition to the inert diluent, the oral composition may further include an adjuvant, such as a wetting agent, an emulsifying and suspending agent, a sweetener, a flavoring agent and a flavoring agent.

Injectable preparations, such as sterile injectable water or oil-based suspensions, can be prepared by known techniques using suitable dispersing or wetting agents and suspending agents. The sterile injectable preparations may also be sterile injectable solutions, suspensions or emulsions in nontoxic parenterally acceptable diluents or solvents, such as solutions in 1,3-butanediol. Among the acceptable vehicles and solvents, water, Ringer's solution, U.S.P and isotonic sodium chloride solution can be used. In addition, sterile and nonvolatile oil is conventionally used as a solvent or suspending medium. To this regard, any odorless nonvolatile oil can be used, including synthetic monoglycerides or diglycerides. In addition, fatty acids, such as octadecenoic acid, are used to prepare the injections.

For example, the injectable preparations can be sterilized by filtering through a bacteria retention filter, or by adding a disinfectant that is in the form of a sterile solid composition and dissolved or dispersed in sterile water or other sterile injectable media before use.

To extend the effect of the compound or composition of the present disclosure, it is often desirable to slow down the absorption of the compound after subcutaneous or intramuscular injection. This can be achieved by using a liquid suspension of a crystalline or amorphous substance with poor water solubility. Then, the absorption rate of the compound depends on the dissolution rate, which in turn depends on the grain size and crystalline form. Alternatively, delayed absorption of the parenterally administered compound can be achieved by dissolving or suspending the compound in an oil vehicle. Injectable depot forms can be prepared by forming a microcapsule matrix of the compound in a biodegradable polymer such as polylactide-polyglycolic acid. The release rate of the compound can be controlled according to the ratio of the compound to the polymer and the properties of particular polymer used. Examples of other biodegradable polymers include polyorthoesters and polyanhydrides. Injectable depot preparations can also be prepared by entrapping the compound in liposome or microemulsion compatible with body tissues.

Compositions for rectal or vaginal administration are particularly suppositories that can be prepared by mixing the compound of the present disclosure with a suitable non-irritating excipient or carrier, such as cocoa butter, polyethylene glycol or suppository wax. The excipient or carrier is solid at ambient temperature but liquid at body temperature, and thus melts and releases the active compound in the rectum or vaginal cavity.

Oral solid dosage forms include capsules, tablets, pills, powders and granules. In such solid dosage forms, the active compound is mixed with at least a pharmaceutically acceptable excipient or carrier such as sodium citrate or dicalcium phosphate, and/or (a) a filler or a bulking agent, such as starch, lactose, sucrose, glucose, mannitol, and silicic acid; (b) a binder, such as carboxymethyl cellulose, alginate, gel, polyvinylpyrrolidone, sucrose, and gum arabic; (c) a humectant, such as glycerol; (d) a disintegrating agent, such as agar, calcium carbonate, potato or tapioca starch, alginic acid, some silicates and/or sodium carbonate; (e) a retarder solution, such as paraffin; (f) an absorption accelerator, such as a quaternary ammonium compound; (g) a wetting agent, such as cetanol and glyceryl monostearate; (h) an absorbent, such as kaolin and bentonite; and (i) a lubricant, such as talc, calcium stearate, magnesium stearate, solid polyethylene glycol, sodium dodecyl sulfate, and a mixture thereof. In the case of capsules, tablets and pills, the dosage forms may also contain a buffer.

Similar type of solid compositions using excipients such as lactose or toffee and high molecular weight polyethylene glycol can also be used as fillers in soft and hard gel capsules. Solid dosage forms such as tablets, lozenges, capsules, pills and granules can be prepared with coatings and shells, such as enteric coatings and other coatings well known in the pharmaceutical field. They may optionally contain an opacifying agent and may also have the properties of a composition, so that only the active ingredient is optionally released in a delayed manner, or preferably released in a certain part of the intestine. Examples of embedding compositions that can be used include polymers and waxes. Similar type of solid compositions including excipients such as lactose or toffee and high molecular weight polyethylene glycol can also be used fillers in soft and hard gel capsules.

The active compound can also be in a micro-seal form with one or more of the above excipients. Solid dosage forms such as tablets, lozenges, capsules, pills and granules can be prepared with coatings and shells, such as enteric coatings, controlled-release coatings and other coatings well known in the pharmaceutical field. In this solid dosage form, the active compound may be mixed with at least one inert diluent, such as sucrose, lactose or starch. Generally, this dosage form may also contain other substances than the inert diluent, for example, a tabletting lubricant and other tabletting auxiliaries, such as magnesium stearate and microcrystalline cellulose. In the case of capsules, tablets and pills, the dosage forms may also contain a buffer. They may optionally contain an opacifying agent and may also have the properties of a composition, so that only the active ingredient is optionally released in a delayed manner, or preferably released in a certain part of the intestine. Examples of embedding compositions that can be used include polymers and waxes.

The topical or transdermal dosage forms of the compound of the present disclosure include ointments, pastes, creams, lotions, gels, powders, solutions, sprays, inhalants or patches. Under aseptic conditions, the active compound is mixed with a pharmaceutically acceptable carrier and any necessary preservative or a possible buffer if needed. Ophthalmic preparations, ear drops and eye drops are also contemplated within the scope of the present disclosure. In addition, the present disclosure contemplates the use of skin patches with the additional advantage of providing controlled delivery of the compound to the body. This dosage form can be prepared by dispersing or dissolving the compound in an appropriate medium. An absorption enhancer can also be used to increase the flux of the compound through the skin. The rate can be controlled by providing a rate-controlling membrane or by dispersing the compound in a polymer matrix or gel.

The composition of the present disclosure can also be administered orally, parenterally, by inhalation of a spray, locally, rectally, nasally, buccally, or vaginally, or by implanting a drug depot. As used in the present disclosure, the term “parenteral” includes, but is not limited to, subcutaneous, intravenous, intramuscular, intraarticular, intrasynovial, intrasternal, intrathecal, intrahepatic, intralesional and intracranial injection or infusion techniques. In particular, the composition is administered orally, intraperitoneally or intravenously.

The sterile injectable form of the composition of the present disclosure can be a water- or oil-based suspension. These suspensions can be prepared by following the techniques known in the art using a suitable dispersing or wetting agent and suspending agent. The sterile injectable preparations may also be sterile injectable solutions or suspensions in nontoxic parenterally acceptable diluents or solvents, such as solutions in 1,3-butanediol. Among the acceptable vehicles and solvents, water, Ringer's solution, and isotonic sodium chloride solution can be used. In addition, sterile and nonvolatile oil is conventionally used as a solvent or suspending medium. To this regard, any odorless nonvolatile oil can be used, including synthetic monoglycerides or diglycerides. In addition, natural pharmaceutically acceptable oils in polyoxyethylated forms such as olive oil or castor oil, and fatty acids such as octadecenoic acid and glyceride derivatives thereof, can be used to prepare injections. These oil-based solutions or suspensions may also contain a long-chain alcohol diluent or dispersant, such as carboxymethyl cellulose or similar dispersants commonly used in preparing pharmaceutically acceptable dosage forms (including emulsions and suspensions). Other commonly used surfactants, such as Tweens, Spans and other emulsifiers or bioavailability enhancers commonly used in the production of pharmaceutically acceptable solid, liquid or other dosage forms, can also be used for preparation purposes.

The pharmaceutical composition of the present disclosure can be taken orally in any orally acceptable dosage forms, including, but not limited, to capsules, tablets, aqueous suspensions or solutions. In the case of tablets for oral administration, common carriers include, but are not limited to, lactose and starch. A lubricant, such as magnesium stearate, is usually added. For oral administration in a capsule form, useful diluents include lactose and dried corn starch. When an aqueous suspension is required for oral administration, the active ingredient is combined with an emulsifier and suspending agent. If necessary, some sweeteners, flavor enhancers or colorants can also be added.

Alternatively, the pharmaceutical composition of the present disclosure can be administered in the form of a suppository for rectal use. These pharmaceutical compositions can be prepared by mixing the reagent and a non-irritating excipient, which is solid at room temperature but liquid at rectal temperature, so they will melt in the rectum to release the drug. Such substances include, but are not limited to, cocoa butter, beeswax and polyethylene glycol.

Especially, when the treatment target includes local drip to accessible areas or organs, including eye, skin or low intestinal diseases, the pharmaceutical composition of the present disclosure can also be locally applied. It is easy to prepare a suitable local preparation for each of these areas or organs.

Local drip to low intestine can be realized by using a rectal suppository (see above) or suitable enteroclysm. Local skin patches can also be used.

For local drip, the pharmaceutical composition can be prepared into a suitable ointment containing the active ingredient suspended or dissolved in one or more carriers. Carriers suitable for local drip of the compound of the present disclosure include, but are not limited to, mineral oil, vaseline oil, white vaseline, propylene glycol, polyoxyethylene, polyoxypropylene, emulsified wax and water. Alternatively, the pharmaceutical composition can be prepared into a suitable lotion or cream containing the active ingredient suspended or dissolved in one or more pharmaceutically acceptable carriers. Suitable carriers include, but are not limited to, mineral oil, sorbitan monostearate, polysorbate 60, cetyl ester wax, cetearyl alcohol, 2-octyl dodecanol, benzyl alcohol and water.

For ophthalmic use, the pharmaceutical composition can be prepared into a micronized suspension in isotonic pH-adjusted sterile saline, or particularly a solution in isotonic pH-adjusted sterile saline, with or without preservatives such as benzalkonium chloride. Alternatively, for ophthalmic use, the pharmaceutical composition can be prepared into an ointment, such as vaseline.

The pharmaceutical composition can also be administered by an aerosol nasal spray or by inhalation. According to the technology well known in the pharmaceutical field, the composition is prepared into a solution in saline by using benzyl alcohol and other suitable preservatives, an absorption promoter for improving the bioavailability, a fluorocarbon compound and/or other conventional solubilizers or dispersants.

The compound used in the method of the present disclosure can be prepared into a unit dosage form. The term “unit dosage form” refers to a physically discrete unit suitable for use as a unit dose for a subject, and each unit contains a predetermined amount of active substance calculated to produce a desired therapeutic effect, optionally in combination with a suitable drug carrier. A unit dosage form can be used as a single daily dose or one of multiple daily doses (for example, about 1-4 times a day or more). When multiple daily doses are used, the unit dosage form may be the same or different for each dose.

### USE OF THE COMPOUND AND COMPOSITION OF THE PRESENT DISCLOSURE

The compound and pharmaceutical composition provided in the present disclosure can be used for preparing drugs for preventing, treating or alleviating diseases related to TTK overexpression or overactivity in patients. Preferably, the diseases related to TTK overexpression or overactivity are tumors, for example, papillary thyroid carcinoma, breast cancer, gastric cancer, bronchial cancer or lung cancer.

The present disclosure also provides use of the compound or pharmaceutical composition in the preparation of drugs for inhibiting TTK.

The present disclosure further provides a method for treating, preventing or delaying diseases caused by TTK overexpression or overactivity, which includes administering a therapeutically effective amount of the compound or pharmaceutical composition to patients in need of treatment. The diseases caused by TTK overexpression or overactivity include papillary thyroid carcinoma, breast cancer, gastric cancer, bronchial cancer or lung cancer. Moreover, the compound or pharmaceutical composition provided in the present disclosure can be co-administered with other therapies or therapeutic agents. The administration can be simultaneously, sequentially or at a certain time interval.

The dosage of the compound or pharmaceutical composition needed for treatment, prevention or delay usually depends on the specific compound to be administered, the patient, the specific disease or disorder and its severity, and the route and frequency of administration, etc., and needs to be determined by the attending physician according to the specific situation. For example, when the compound or pharmaceutical composition provided in the present disclosure is administered by intravenous injection, it can be administered once a week or even at longer intervals.

To sum up, the present disclosure provides a novel compound which can be used as is a TTK inhibitor. The compound of the present disclosure is suitable to be prepared into various dosage forms, and can be widely used for treating tumors, such as papillary thyroid cancer, breast cancer, gastric cancer, bronchial cancer or lung cancer.

The compound and pharmaceutical composition of the present disclosure are not only beneficial to human treatment, but also can be applied to veterinary treatment of pets, imported animals and mammals in farm animals. Other examples of animals include horses, dogs and cats. Here, the compound of the present disclosure includes its pharmaceutically acceptable derivatives.

General Synthesis Process

To describe the present disclosure, examples are listed below. However, it should be understood that these examples are not intended to limit the present disclosure, but provides methods for practicing the invention.

Generally, the compound of the present disclosure can be prepared by the method described in the present disclosure, where the definitions of substituents are described for those in Formula (I), unless otherwise specified. The following reaction schemes and examples are used to further illustrate the present disclosure by way of examples.

Those skilled in the art will realize that the chemical reactions described in the present disclosure can be used to prepare many other compounds of the present disclosure appropriately, and other methods for preparing the compounds of the present disclosure are contemplated in the scope of the present disclosure. For example, the synthesis of those non-exemplified compounds according to the present disclosure can be successfully accomplished by those skilled in the art through modifications, such as appropriate protection of interfering groups, use of other known reagents than those described in the present disclosure, or some conventional modifications to the reaction conditions. In addition, the reaction disclosed in the present disclosure or the known reaction conditions are also generally applicable to the preparation of other compounds in the present disclosure.

In the examples described below, the temperatures are degrees Celsius, unless otherwise indicated. The solvents used in the present disclosure are commercially available. The reagents are purchased from commercial suppliers such as Aldrich Chemical Company, Arco Chemical Company and Alfa Chemical Company, J&K Scientific Ltd., and will not be further purified when used, unless otherwise indicated.

The LCMS model used in the following examples is Agilent Technologies 6110, and the NMR spectrometer model is Avance III 400 MHz.

The following abbreviations are used in the present disclosure: DIPEA represents N-diisopropylethylamine; HOAT represents 1-hydroxy-7-azobenzotriazole; DMF represents N,N-dimethyl acetamide; EDCI represents 1-(3-dimethylaminopropyl)-3-ethylcarbodiimide hydrochloride; DCE represents 1,2-dichloroethane; BINAP represents 1,1′-binaphthyl-2,2′-bis(diphenylphosphine); NMP represents N-methyl pyrrolidone; and m-CPBA represents m-chloroperoxybenzoic acid.

The compound is named according to the conventional naming rules in the art or by ChemDraw software.

The experimental steps for preparing the compound disclosed in the present disclosure are listed in the following synthesis schemes. In the compound, R₁, R₂, R₃, R₄, R₅, R₆, L₂, R′, m, X, ring Y, ring V, ring W, R^{Y}, R^{w}, R^{V}, q, s, and p have the meanings as defined herein,

Synthesis Scheme 1

Compound II-a is subjected to a nucleophilic substitution reaction with the nucleophilic reagent X—H in the presence of a base, to obtain Compound II-b. The Compound II-b is coupled with a corresponding aniline compound in the presence of a metal catalyst to prepare Compound II-B.

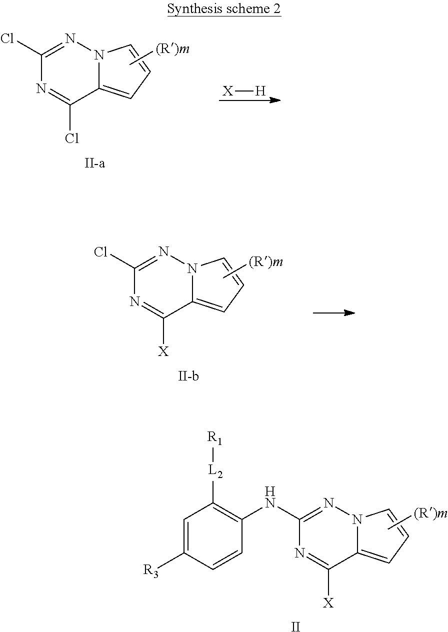

Compound III-a is oxidized by m-CPBA to obtain Compound III-b, then III-b is subjected to a nucleophilic substitution reaction with a corresponding carboxamide compound in the presence of a base, and then undergoes alkaline hydrolysis to obtain Compound III-C. Compound III-c is coupled with a corresponding boric acid or boron ester compound in the presence of a metal to obtain a compound of Formula III.

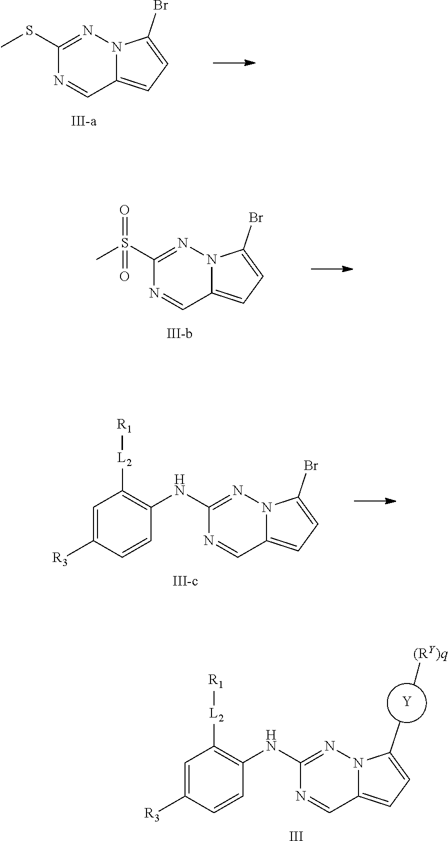

### DESCRIPTION OF EMBODIMENTS

The following examples are intended to illustrate the present disclosure, instead of limiting the scope of the present disclosure.

### PREPARATION EXAMPLES

In the following preparation examples, the preparation process of the compound of the present disclosure is described in detail by taking some compounds of the present disclosure as examples.

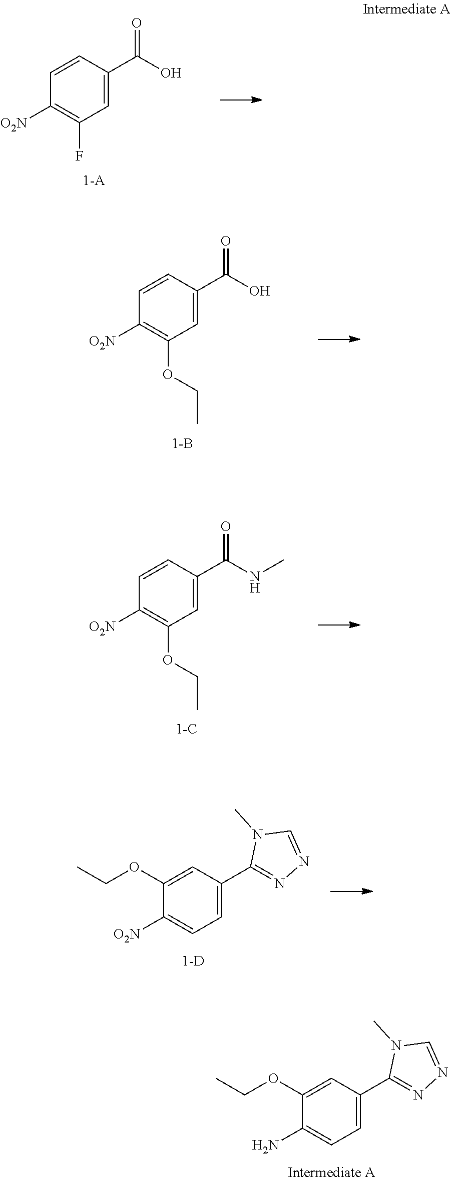 [SMILES: O=C(O)c1ccc([N+](=O)[O-])c(F)c1 ; CCOc1cc(C(=O)O)ccc1[N+](=O)[O-] ; CCOc1cc(C(=O)NC)ccc1[N+](=O)[O-] ; CCOc1cc(-c2nncn2C)ccc1[N+](=O)[O-] ; CCOc1cc(-c2nncn2C)ccc1N]

Step I.

Compound 1-A (5.3 g, 28.7 mmol) was dissolved in EtOH (50 mL), and then EtONa (4.7 g, 68.8 mmol) was added to the reaction system. The reaction mixture was heated to reflux for 2 h. The reaction system was diluted with water, and then 2N HCl was added until a large amount of solid was precipitated out. After filtration under suction, the filter cake was washed with water (3*40 mL), and dried to obtain Compound 1-B as a yellow solid. LCMS (M+H)⁺=212.1.

Step II

Compound 1-B (5.9 g, 28 mmol), DIPEA (18 g, 140 mmol), methylamine hydrochloride (5.7 g, 84 mmol) and HOAT (4.5 g, 34 mmol) were dissolved in DMF (60 mL). EDCI (6.4 g, 34 mmol) was added to the reaction system at room temperature, and reacted at 50° C. for 18 h under nitrogen atmosphere. A 1 N NaOH (80 mL) solution was added to the reaction system. The reaction system was extracted with dichloromethane (3*60 mL), dried over anhydrous sodium sulfate, rotary evaporated to dryness, and purified by column chromatography (PE:EA=5:1) to prepare Compound 1-C as ayellow solid. LCMS (M+H)⁺=225.0.

Step III

Compound 1-C (0.7 g, 3.1 mmol) was dissolved in DCE (20 mL), and then SOCl₂ (2.1 g, 15.6 mmol) was added to the reaction system at room temperature. The reaction mixture was heated to 85° C. and reacted for 2 h under microwave. The reaction mixture was evaporated to dryness under reduced pressure and then dissolved in DMF (15 mL). Then formylhydrazine (281 mg, 4.7 mmol) was added to the reaction system. The reaction mixture was heated to 110° C. and reacted for 1 h. The reaction solution was cooled to room temperature. A large amount of solid was precipitated out by adding saturated saline into the reaction system. After filtration, the filter cake was washed with a small amount of water and dried to obtain Compound 1-D as a yellow solid. LCMS (M+H)⁺=249.0.

Step IV

Compound 1-D (300 mg, 1.2 mmol) was dissolved in EtOH (15 mL)/MeOH (5 mL), and then 10% Pd/C (130 mg) was added to the reaction system. The reaction mixture was heated to 70° C. and reacted for 12 h under hydrogen atmosphere. After filtration under suction, the filter cake was washed, and the filtrate was rotary evaporation to dryness to obtain Intermediate A. LCMS (M+H)⁺=219.0. ¹H NMR (400 MHz, DMSO) δ 8.53-8.35 (m, 1H), 7.11 (d, J=1.7 Hz, 1H), 7.04 (dd, J=8.1, 1.7 Hz, 1H), 6.72 (t, J=9.4 Hz, 1H), 5.16 (s, 2H), 4.14-3.95 (m, 2H), 3.76-3.63 (m, 3H), 1.47-1.23 (m, 3H).

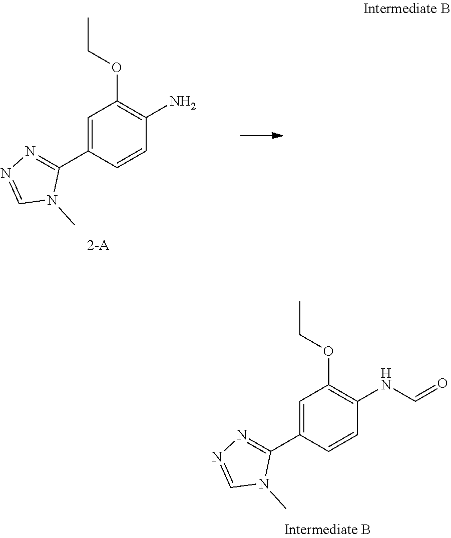 [SMILES: CCOc1cc(-c2nncn2C)ccc1N ; CCOc1cc(-c2nncn2C)ccc1NC=O]

Step I:

Compound 2-A (200 mg, 0.9 mmol) was dissolved in formic acid (10 mL), and then solid sodium formate (1.2 g, 18 mmol) wad added. The reaction solution was heated to 70° C., and stirred for 1 h. Most formic acid was removed by concentrated under reduced pressure. The residue was adjusted to pH 9-10 with a saturated aqueous sodium bicarbonate solution, then extracted with dichloromethane (3*40 mL), dried over anhydrous sodium sulfate, rotary evaporated to dryness, and purified by column chromatography (PE:EA=2:1), to prepare Intermediate B as a yellow solid. LCMS: (M+H)⁺=247.0.

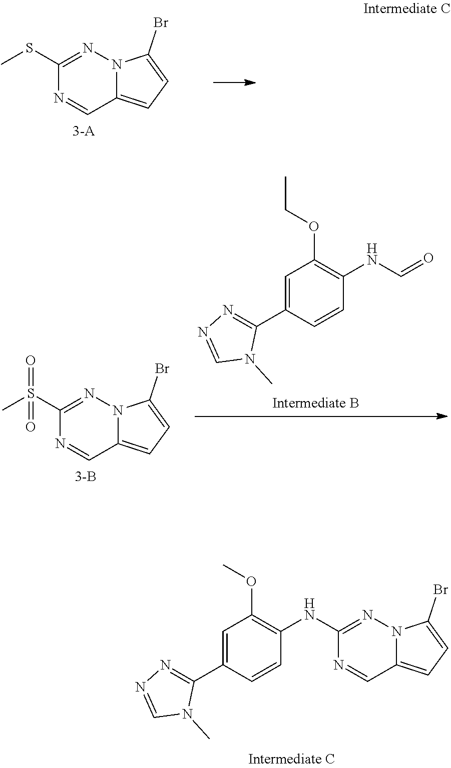

Step I:

Compound 3-A (200 mg, 0.8 mmol) was dissolved in tetrahydrofuran (5 mL), m-chloroperoxybenzoic acid (987 mg, 5.7 mmol) was added in batches in an ice bath, and the reaction solution was heated to 50° C. and stirred for 2 h. m-chloroperoxybenzoic acid (100 mg, 0.57 mmol) was further added in batches in an ice-water bath, and reacted for another 2 h. The solvent was removed by concentration. The residue was diluted with ethyl acetate (30 mL), washed with an aqueous sodium bicarbonate solution and then washed with saturated saline. The organic phase was dried over anhydrous sodium sulfate, and concentrated. The crude product was purified by column chromatography (PE:EA=3:7) to obtain Compound 3-B as a yellow solid. LCMS (M+H)⁺=276.0.

Step II

Intermediate B (50 mg, 0.2 mmol) was dissolved in THF (5 mL), NaH (20 mg, 0.8 mmol) was added in batches in an ice-water bath, and the reaction solution was heated to 50° C. and stirred for 1 h. After cooling to room temperature, Compound 3-B (56 mg, 0.2 mmol) was added in batches, and reacted at 50° C. for another 12 h. After cooling to room temperature, a 2 N aqueous sodium hydroxide solution (0.5 mL) and methanol (0.5 mL) were added, and reacted at room temperature for 1 h. The reaction solution was concentrated, and the crude product was separated by HPLC to obtain Intermediate C. LCMS: (M+H)⁺=414.0. ¹H NMR (400 MHz, DMSO) δ 8.97 (s, 1H), 8.58 (d, J=11.3 Hz, 2H), 8.04 (s, 1H), 7.45-7.31 (m, 2H), 7.01 (dd, J=20.9, 4.6 Hz, 2H), 4.25 (q, J=7.0 Hz, 2H), 3.79 (s, 3H), 1.44 (t, J=6.9 Hz, 3H).

### Example 1

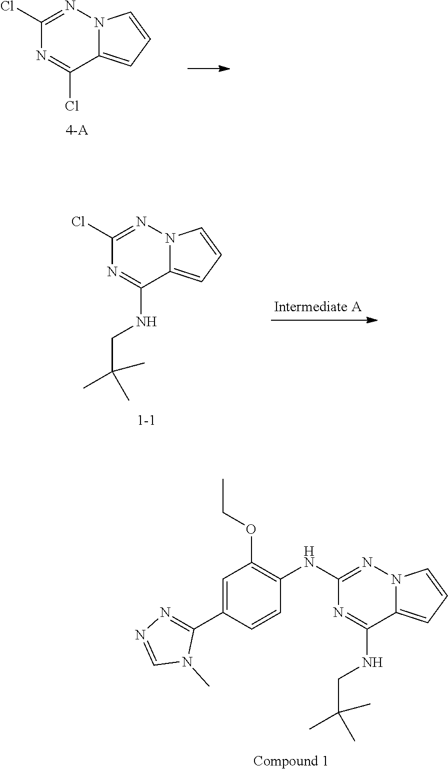 [SMILES: Clc1nc(Cl)c2cccn2n1 ; CC(C)(C)CNc1nc(Cl)nn2cccc12 ; CCOc1cc(-c2nncn2C)ccc1Nc1nc(NCC(C)(C)C)c2cccn2n1]

Step I:

Compound 4-A (200 mg, 1 mmol), DIPEA (332 mg, 2.6 mmol) and 2,2-methylpropan-1-amine (112 mg, 1.3 mmol) were dissolved in acetonitrile (5 mL), and the reaction solution was heated to 60° C. for 12 h. The reaction solution was directly concentrated into a yellow oil, and the crude product was purified by column chromatography (PE:EA=1:1) to obtain Compound 1-1 as a yellow oil. LCMS: (M+H)⁺=239.0.

Step II

Compound 1-1 (98 mg, 0.4 mmol), Intermediate A (60 mg, 0.275 mmol), BINAP (34 mg, 0.055 mmol), Cs₂CO₃ (224 mg, 0.688 mmol) and palladium acetate (6.0 mg, 0.028 mmol) were dissolved in dioxane (10 mL), and the reaction mixture was heated to 100° C., and reacted for 12 h under nitrogen atmosphere. The reaction solution was concentrated, and the crude product was separated by HPLC to obtain Compound 1. LCMS: (M+H)⁺=421.0. ¹H NMR (400 MHz, DMSO) δ 8.59-8.48 (m, 2H), 8.08 (t, J=6.2 Hz, 1H), 7.55-7.47 (m, 1H), 7.37-7.27 (m, 2H), 7.19 (s, 1H), 6.97 (dd, J=4.4, 1.6 Hz, 1H), 6.48 (dd, J=4.3, 2.5 Hz, 1H), 4.24 (q, J=6.9 Hz, 2H), 3.76 (s, 3H), 3.47-3.26 (m, 4H), 1.44 (t, J=6.9 Hz, 3H), 0.97 (s, 9H).

### Example 2

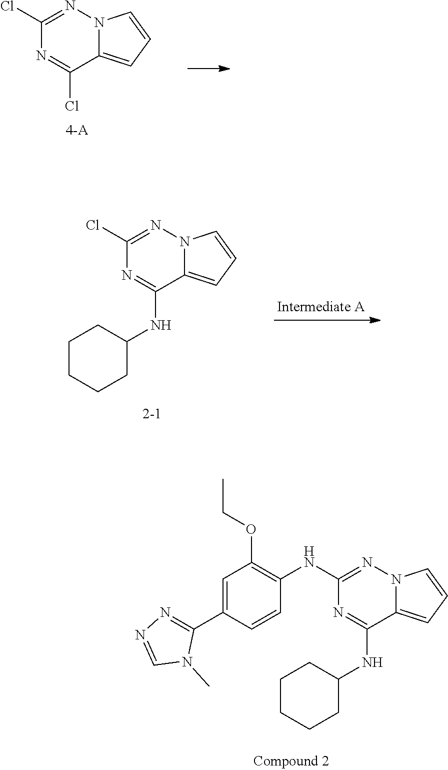 [SMILES: Clc1nc(Cl)c2cccn2n1 ; Clc1nc(NC2CCCCC2)c2cccn2n1 ; CCOc1cc(-c2nncn2C)ccc1Nc1nc(NC2CCCCC2)c2cccn2n1]

Step L:

Compound 4-A (250 mg, 1.3 mmol) was dissolved in acetonitrile (5 mL), and cyclohexylamine (160 mg, 1.6 mmol) and DIPEA were added to the reaction system. The mixture was heated to 60° C., reacted for 16 h, and concentrated to remove the solvent. The crude product was purified by column chromatography (PE:EA=1:1) to obtain Compound 2-1 as a yellow solid. LCMS (M+H)⁺=251.0.

Step II

Compound 2-1 (172 mg, 0.7 mmol), Intermediate A (100 mg, 0.5 mmol), BINAP (57 mg, 0.09 mmol), Cs₂CO₃ (374 mg, 1.1 mmol) and palladium acetate (10 mg, 0.05 mmol) were dissolved in dioxane (10 mL), and the reaction mixture was heated to 100° C., and reacted for 12 h under nitrogen atmosphere. The reaction solution was concentrated, and the crude product was separated by HPLC to obtain Compound 2. LCMS: (M+H)⁺=433.0.

¹H NMR (400 MHz, DMSO) δ 8.61-8.48 (m, 2H), 7.97 (d, J=7.8 Hz, 1H), 7.49 (s, 1H), 7.37-7.27 (m, 2H), 7.21 (s, 1H), 6.89 (d, J=2.8 Hz, 1H), 6.46 (dd, J=4.3, 2.5 Hz, 1H), 4.24 (q, J=7.0 Hz, 2H), 4.08 (m, 1H), 3.77 (m, 3H), 1.99 (m, 2H), 1.79 (m, 2H), 1.67 (d, J=14.4 Hz, 1H), 1.45 (t, J=6.9 Hz, 3H), 1.38 (m, 4H), 1.20 (m, 1H).

### Example 3

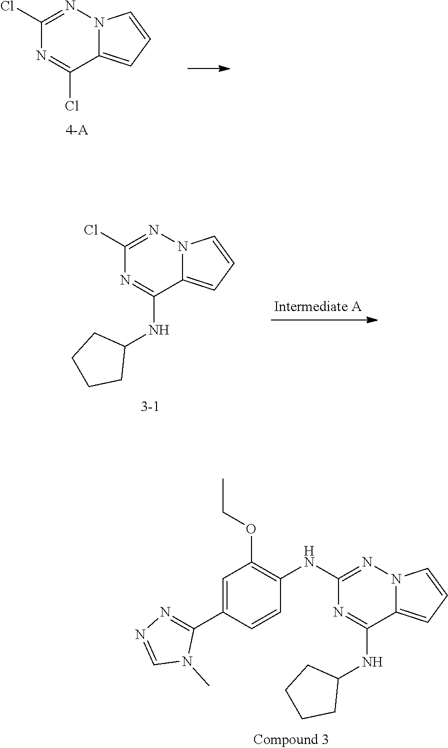 [SMILES: Clc1nc(Cl)c2cccn2n1 ; Clc1nc(NC2CCCC2)c2cccn2n1 ; CCOc1cc(-c2nncn2C)ccc1Nc1nc(NC2CCCC2)c2cccn2n1]

Step I:

Compound 4-A (500 mg, 2.7 mmol) was dissolved in NMP (5 mL), and then DIPEA (690 mg, 5.3 mmol) and cyclopentanamine (455 mg, 5.3 mmol) were added to the reaction system. The reaction solution was heated to 120° C. and stirred for 1 h. The reaction system was cooled to room temperature, and then the reaction was quenched with water, extracted with ethyl acetate (3*40 mL), and washed with saturated saline. The organic phase was dried over anhydrous sodium sulfate, and rotary evaporated to dryness. The crude product was purified by column chromatography (PE:EA=1:1) to obtain Compound 3-1 as a yellow solid. LCMS (M+H)⁺=237.1.

Step II

Compound 3-1 (100 mg, 0.4 mmol), Intermediate A (139 mg, 0.6 mmol), BINAP (53 mg, 0.09 mmol) and Cs₂CO₃ (346 mg, 1 mmol) were dissolved in dioxane (10 mL). Palladium acetate (10 mg, 0.04 mmol) was added to the reaction system at room temperature, and the reaction was continued at 110° C. for 18 h under nitrogen atmosphere. The solvent was removed by concentration and the crude product was separated by HPLC to obtain Compound 3. LCMS (M+H)⁺=419.0. ¹H NMR (400 MHz, DMSO) δ 8.54 (t, J=4.1 Hz, 2H), 8.02 (d, J=7.3 Hz, 1H), 7.50 (s, 1H), 7.31 (dd, J=12.5, 4.1 Hz, 2H), 7.20 (s, 1H), 6.90 (dd, J=4.3, 1.3 Hz, 1H), 6.46 (dd, J=4.1, 2.6 Hz, 1H), 4.53 (dd, J=13.8, 7.0 Hz, 1H), 4.25 (q, J=6.9 Hz, 2H), 3.78 (d, J=11.1 Hz, 3H), 2.03 (d, J=2.6 Hz, 2H), 1.76 (s, 2H), 1.63 (d, J=19.8 Hz, 4H), 1.44 (t, J=6.9 Hz, 3H).

### Example 4

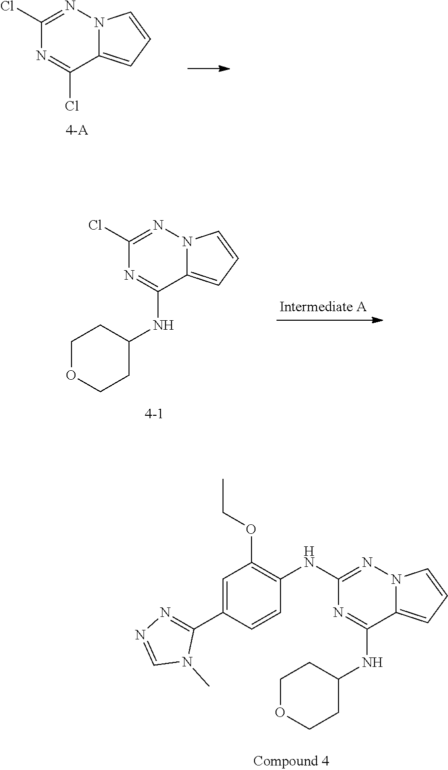 [SMILES: Clc1nc(Cl)c2cccn2n1 ; Clc1nc(NC2CCOCC2)c2cccn2n1 ; CCOc1cc(-c2nncn2C)ccc1Nc1nc(NC2CCOCC2)c2cccn2n1]

Step I:

Compound 4-A (500 mg, 2.7 mmol) was dissolved in NMP (5 mL), and then DIPEA (690 mg, 5.3 mmol) and tetrahydro-2H-pyran-4-amine (540 mg, 5.3 mmol) were added to the reaction system. The reaction solution was heated to 120° C. and stirred for 1 h. The reaction system was cooled to room temperature, and then the reaction was quenched with water, extracted with ethyl acetate (3*40 mL), and washed with saturated saline. The organic phase was dried over anhydrous sodium sulfate, and rotary evaporated to dryness. The crude product was purified by column chromatography (PE:EA=1:1) to obtain Compound 4-1 as a yellow solid. LCMS (M+H)⁺=253.0.

Step II

Compound 4-1 (100 mg, 0.4 mmol), Intermediate A (130 mg, 0.6 mmol), BINAP (50 mg, 0.08 mmol) and Cs₂CO₃ (324 mg, 1 mmol) were dissolved in dioxane (10 mL). Palladium acetate (9 mg, 0.04 mmol) was added to the reaction system at room temperature, and the reaction was continued at 110° C. for 18 h under nitrogen atmosphere. The solvent was removed by concentration and the crude product was separated by HPLC to obtain Compound 4. LCMS (M+H)⁺=435.0. ¹H NMR (400 MHz, DMSO) δ 8.59-8.44 (m, 2H), 8.04 (d, J=7.7 Hz, 1H), 7.51 (s, 1H), 7.39-7.29 (m, 2H), 7.24 (s, 1H), 6.89 (d, J=4.2 Hz, 1H), 6.54-6.41 (m, 1H), 4.41-4.29 (m, 1H), 4.24 (q, J=6.9 Hz, 2H), 3.94 (d, J=8.1 Hz, 2H), 3.80-3.73 (m, 3H), 3.46 (t, J=11.6 Hz, 2H), 1.92 (d, J=10.9 Hz, 2H), 1.63 (qd, J=12.3, 4.5 Hz, 2H), 1.44 (t, J=6.9 Hz, 3H).

### Example 5

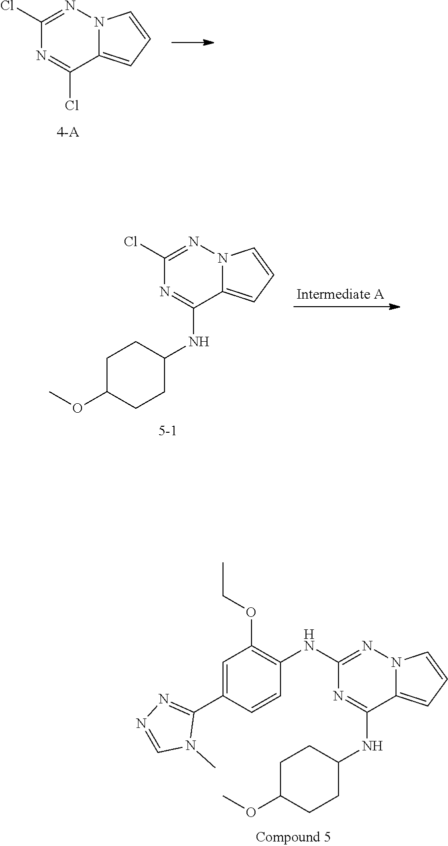 [SMILES: Clc1nc(Cl)c2cccn2n1 ; COC1CCC(Nc2nc(Cl)nn3cccc23)CC1 ; CCOc1cc(-c2nncn2C)ccc1Nc1nc(NC2CCC(OC)CC2)c2cccn2n1]

Step I:

Compound 4-A (300 mg, 1.6 mmol) was dissolved in NMP (5 mL), and then DIPEA (414 mg, 3.2 mmol) and 4-methoxycyclohexan-1-amine (414 mg, 3.2 mmol) were added to the reaction system. The reaction solution was heated to 120° C. and stirred for 1 h. The reaction system was cooled to room temperature, and then the reaction was quenched with water, extracted with ethyl acetate (3*40 mL), and washed with saturated saline. The organic phase was dried over anhydrous sodium sulfate, and rotary evaporated to dryness. The crude product was purified by column chromatography (PE:EA=1:1) to obtain Compound 5-1 as a yellow solid. LCMS (M+H)⁺=281.0.

Step II

Compound 5-1 (120 mg, 0.4 mmol), Intermediate A (139 mg, 0.6 mmol), BINAP (53 mg, 0.09 mmol) and Cs₂CO₃ (346 mg, 1 mmol) were dissolved in dioxane (10 mL). Palladium acetate (10 mg, 0.04 mmol) was added to the reaction system at room temperature, and the reaction was continued at 110° C. for 18 h under nitrogen atmosphere. The solvent was removed by concentration and the crude product was separated by HPLC to obtain Compound 5. LCMS (M+H)⁺=463.0. ¹H NMR (400 MHz, DMSO) δ 8.58-8.46 (m, 2H), 7.98 (d, J=7.8 Hz, 1H), 7.52-7.46 (m, 1H), 7.35-7.26 (m, 2H), 7.23 (s, 1H), 6.88 (dd, J=4.4, 1.6 Hz, 1H), 6.46 (dd, J=4.3, 2.5 Hz, 1H), 4.24 (q, J=6.9 Hz, 2H), 4.17-4.00 (m, 1H), 3.78 (d, J=10.3 Hz, 3H), 3.25 (d, J=11.5 Hz, 3H), 3.22-3.07 (m, 1H), 2.05 (dd, J=29.4, 10.0 Hz, 4H), 1.51-1.37 (m, 5H), 1.28 (dd, J=23.0, 10.2 Hz, 2H).

### Example 6

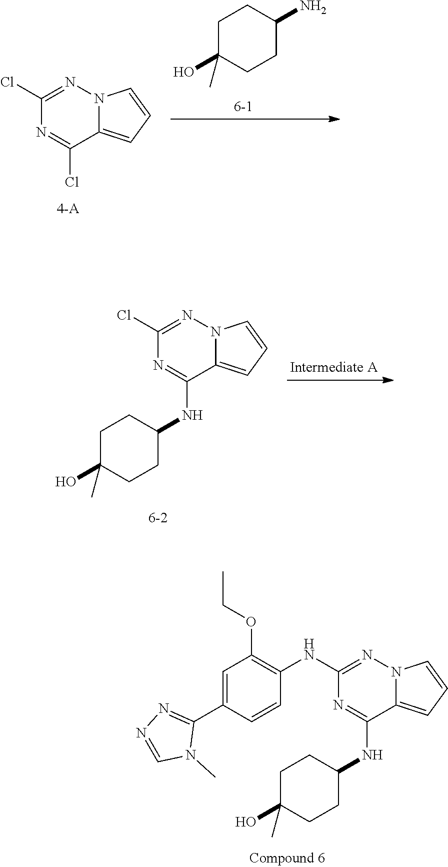

Step I:

Compound 4-A (100 mg, 0.8 mmol) was dissolved in NMP (5 mL), and then DIPEA (200 mg, 1.5 mmol) and Compound 6-1 (160 mg, 0.85 mmol) were added to the reaction system. The reaction solution was heated to 120° C. and stirred for 1 h. The reaction system was cooled to room temperature, and then the reaction was quenched with water, extracted with ethyl acetate (3*30 mL), and washed with saturated saline. The organic phase was dried over anhydrous sodium sulfate, and rotary evaporated to dryness. The crude product was purified by column chromatography (PE:EA=1:1) to obtain Compound 6-2 as a yellow solid. LCMS (M+H)⁺=281.1.

Step II

Compound 6-2 (70 mg, 0.25 mmol), Intermediate A (55 mg, 0.25 mmol), BINAP (31 mg, 0.05 mmol) and Cs₂CO₃ (204 mg, 0.6 mmol) were dissolved in dioxane (10 mL). Palladium acetate (6 mg, 0.03 mmol) was added to the reaction system at room temperature, and the reaction was continued at 110° C. for 18 h under nitrogen atmosphere. The solvent was removed by concentration and the crude product was separated by HPLC to obtain Compound 6. LCMS (M+H)⁺=463.0. ¹H NMR (400 MHz, DMSO) δ 8.59-8.48 (m, 2H), 8.00 (d, J=8.0 Hz, 1H), 7.52-7.45 (m, 1H), 7.39-7.25 (m, 2H), 7.20 (s, 1H), 6.92 (dd, J=4.3, 1.5 Hz, 1H), 6.45 (dd, J=4.3, 2.5 Hz, 1H), 4.24 (q, J=6.9 Hz, 2H), 4.14 (s, 1H), 4.10-4.00 (m, 1H), 3.75 (d, J=11.9 Hz, 3H), 1.87-1.58 (m, 6H), 1.43 (q, J=6.9 Hz, 5H), 1.15 (s, 3H).

### Example 7

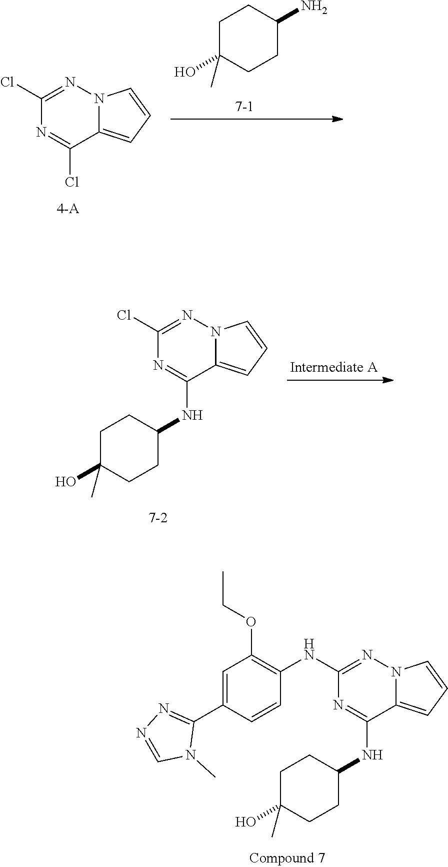

Step I:

Compound 4-A (20 mg, 0.16 mmol) was dissolved in NMP (3 mL), and then DIPEA (40 mg, 0.3 mmol) and Compound 7-1 (32 mg, 0.17 mmol) were added to the reaction system. The reaction solution was heated to 120° C. and stirred for 1 h. The reaction system was cooled to room temperature, and then the reaction was quenched with water, extracted with ethyl acetate (3*30 mL), and washed with saturated saline. The organic phase was dried over anhydrous sodium sulfate, and rotary evaporated to dryness. The crude product was purified by column chromatography (PE:EA=1:1) to obtain Compound 7-2 as a yellow solid. LCMS (M+H)⁺=281.1.

Step II

Compound 7-2 (28 mg, 0.1 mmol), Intermediate A (33 mg, 0.15 mmol), BINAP (13 mg, 0.02 mmol) and Cs₂CO₃ (82 mg, 0.25 mmol) were dissolved in dioxane (10 mL). Palladium acetate (3 mg, 0.01 mmol) was added to the reaction system at room temperature, and the reaction was continued at 110° C. for 18 h under nitrogen atmosphere. The solvent was removed by concentration and the crude product was separated by HPLC to obtain Compound 7. LCMS (M+H)⁺=463.0. ¹H NMR (400 MHz, DMSO) δ 8.52 (d, J=9.5 Hz, 2H), 7.91 (d, J=7.8 Hz, 1H), 7.49 (s, 1H), 7.37-7.27 (m, 2H), 7.21 (s, 1H), 6.88 (dd, J=4.3, 1.5 Hz, 1H), 6.47 (dd, J=4.3, 2.5 Hz, 1H), 4.38 (s, 1H), 4.24 (q, J=7.0 Hz, 2H), 4.11 (s, 1H), 3.77 (s, 3H), 3.32 (s, 2H), 1.90 (s, 2H), 1.65 (d, J=4.4 Hz, 2H), 1.55 (dd, J=19.1, 11.2 Hz, 4H), 1.45 (t, J=6.9 Hz, 3H), 1.19 (d, J=7.6 Hz, 3H).

### Example 8

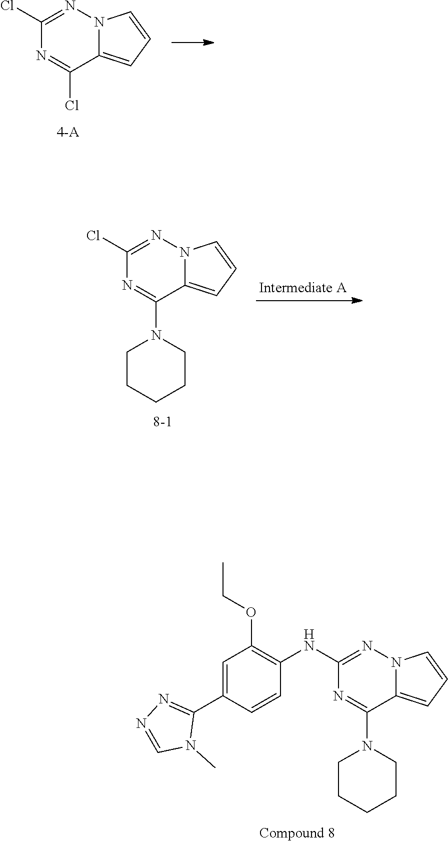 [SMILES: Clc1nc(Cl)c2cccn2n1 ; Clc1nc(N2CCCCC2)c2cccn2n1 ; CCOc1cc(-c2nncn2C)ccc1Nc1nc(N2CCCCC2)c2cccn2n1]

Step L:

Compound 4-A (300 mg, 1.6 mmol) was dissolved in NMP (5 mL), and then DIPEA (414 mg, 3.2 mmol) and piperidine (389 mg, 3.2 mmol) were added to the reaction system. The reaction solution was heated to 120° C. and stirred for 1 h. The reaction system was cooled to room temperature, and then the reaction was quenched with water, extracted with ethyl acetate (3*30 mL), and washed with saturated saline. The organic phase was dried over anhydrous sodium sulfate, and rotary evaporated to dryness. The crude product was purified by column chromatograph (PE:EA=2:1) to obtain Compound 8-1 as a yellow solid. LCMS (M+H)⁺=237.0.

Step II

Compound 8-1 (100 mg, 0.4 mmol), Intermediate A (139 mg, 0.6 mmol), BINAP (53 mg, 0.09 mmol) and Cs₂CO₃ (346 mg, 1 mmol) were dissolved in dioxane (10 mL). Palladium acetate (10 mg, 0.04 mmol) was added to the reaction system at room temperature, and the reaction was continued at 110° C. for 18 h under nitrogen atmosphere. The solvent was removed by concentration and the crude product was separated by HPLC to obtain Compound 8. LCMS (M+H)⁺=419.0. ¹H NMR (400 MHz, DMSO) δ 8.58-8.44 (m, 2H), 7.62 (dd, J=2.5, 1.5 Hz, 1H), 7.37-7.27 (m, 2H), 7.20 (s, 1H), 6.87 (dd, J=4.6, 1.5 Hz, 1H), 6.56 (dd, J=4.6, 2.6 Hz, 1H), 4.24 (q, J=6.9 Hz, 2H), 3.95 (d, J=5.1 Hz, 4H), 3.77 (s, 3H), 1.78-1.59 (m, 6H), 1.44 (t, J=6.9 Hz, 3H).

### Example 9

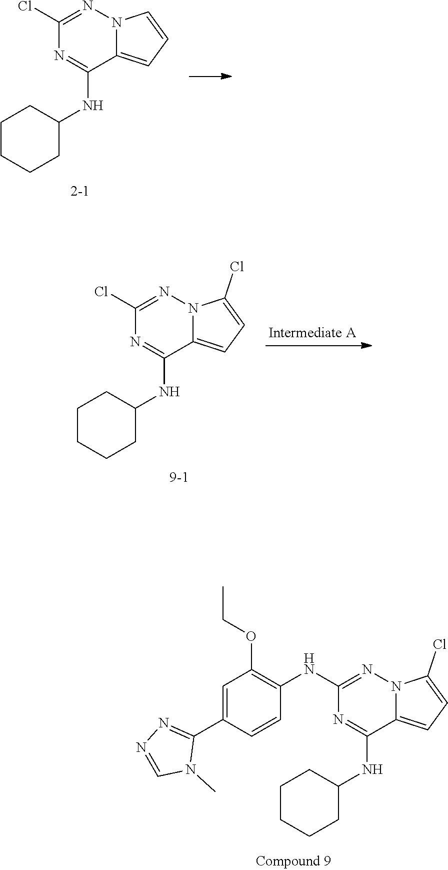 [SMILES: Clc1nc(NC2CCCCC2)c2cccn2n1 ; Clc1nc(NC2CCCCC2)c2ccc(Cl)n2n1 ; CCOc1cc(-c2nncn2C)ccc1Nc1nc(NC2CCCCC2)c2ccc(Cl)n2n1]

Step I:

Compound 2-1 (50 mg, 0.2 mmol) was dissolved in acetonitrile (5 mL), and then NCS (45.5 mg, 0.3 mmol) was added in batches. The reaction solution was heated to 70° C., stirred for 12 h, and concentrated under reduced pressure. The crude product was purified by HPLC to obtain Compound 9-1 as a white solid. LCMS (M+H)⁺=285.0. ¹H NMR (400 MHz, DMSO) δ 8.62 (d, J=8.0 Hz, 1H), 7.12 (d, J=4.7 Hz, 1H), 6.72 (d, J=4.6 Hz, 1H), 4.04 (s, 1H), 2.08 (s, 1H), 1.92 (s, 2H), 1.76 (s, 2H), 1.64 (d, J=12.2 Hz, 1H), 1.35 (t, J=9.8 Hz, 3H), 1.16 (d, J=12.3 Hz, 1H).

Step II

Compound 9-1 (15 mg, 0.05 mmol), BINAP (12 mg, 0.05 mmol), Intermediate A (12 mg, 0.05 mmol) and cesium carbonate (43 mg, 0.13 mmol) were dissolved in dioxane (5 mL), and palladium acetate (1 mg, 0.005 mmol) was added under nitrogen atmosphere. The reaction system was heated to 100° C., and reacted for 12 h under nitrogen atmosphere. The solvent was removed by concentration under reduced pressure and the crude product was separated by HPLC to obtain Compound 9. LCMS (M+H)⁺=467.0. ¹H NMR (400 MHz, DMSO) δ 8.61 (d, J=8.4 Hz, 1H), 8.53 (s, 1H), 8.10 (d, J=8.1 Hz, 1H), 7.33 (dd, J=12.6, 7.6 Hz, 3H), 7.00 (d, J=4.6 Hz, 1H), 6.56 (d, J=4.6 Hz, 1H), 4.26 (q, J=6.8 Hz, 2H), 4.09 (m, 1H), 3.77 (m, 3H), 1.99 (m, 2H), 1.79 (m, 2H), 1.67 (d, J=12.7 Hz, 1H), 1.46 (d, J=6.9 Hz, 3H), 1.37 (m, 3H), 1.23 (m, 2H).

### Example 10

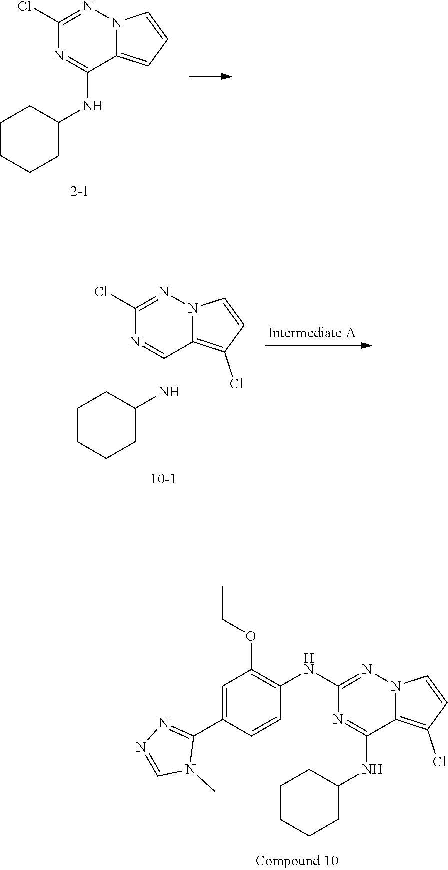

Step I.

Compound 2-1 (50 mg, 0.2 mmol) was dissolved in acetonitrile (5 mL), and NCS (45.5 mg, 0.3 mmol) was added in batches. The reaction solution was heated to 70° C., stirred for 12 h, and concentrated under reduced pressure to obtain a crude product. The crude product was purified by HPLC to obtain Compound 10-1 as a white solid. LCMS (M+H)⁺=285.0. ¹H NMR (400 MHz, DMSO) δ 7.73 (d, J=2.9 Hz, 1H), 7.00 (d, J=8.1 Hz, 1H), 6.75 (d, J=2.9 Hz, 1H), 4.17-3.98 (m, 1H), 1.90 (dd, J=12.3, 3.3 Hz, 2H), 1.72 (dd, J=9.2, 3.9 Hz, 2H), 1.64-1.55 (m, 1H), 1.54-1.45 (m, 2H), 1.36 (td, J=12.0, 3.3 Hz, 2H), 1.20 (td, J=11.9, 3.4 Hz, 1H).

Step II

Compound 10-1 (15 mg, 0.05 mmol), BINAP (6 mg, 0.01 mmol), Intermediate A (12 mg, 0.05 mmol), and cesium carbonate (43 mg, 0.13 mmol) were dissolved in dioxane (5 mL), and then palladium acetate (1 mg, 0.005 mmol) was added. The reaction solution was heated to 100° C., and reacted for 12 h under nitrogen atmosphere. The solvent was removed by concentration under reduced pressure and the crude product was separated by HPLC to obtain Compound 10. LCMS (M+H)⁺=467.0. ¹H NMR (400 MHz, DMSO) δ 8.52 (d, J=15.9 Hz, 1H), 8.41 (d, J=8.4 Hz, 1H), 7.58 (d, J=2.8 Hz, 1H), 7.40-7.24 (m, 3H), 6.66-6.54 (m, 2H), 4.24 (q, J=7.0 Hz, 2H), 4.13 (s, 1H), 3.76 (m, 3H), 1.97 (m, 2H), 1.73 (m, 2H), 1.60 (m, 1H), 1.50 (d, J=12.2 Hz, 2H), 1.43 (t, J=6.9 Hz, 3H), 1.41-1.35 (m, 1H), 1.23 (m, 2H).

### Example 11

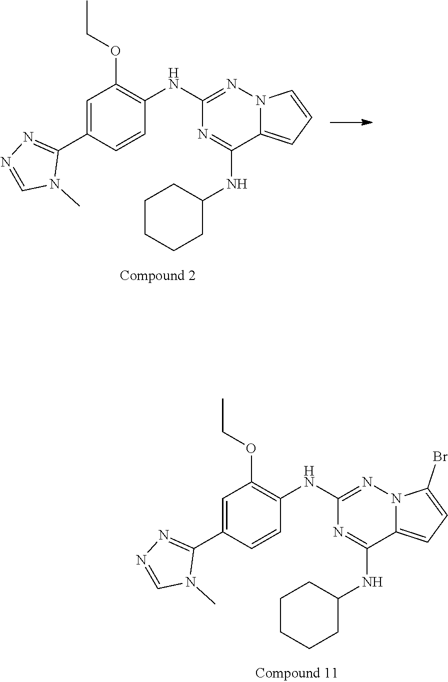 [SMILES: CCOc1cc(-c2nncn2C)ccc1Nc1nc(NC2CCCCC2)c2cccn2n1 ; CCOc1cc(-c2nncn2C)ccc1Nc1nc(NC2CCCCC2)c2ccc(Br)n2n1]

Step I.

Compound 2 (40 mg, 0.09 mmol) was dissolved in acetonitrile (3 mL). NBS (16.5 mg, 0.09 mmol) was dissolved in acetonitrile (2 mL), and then slowly added dropwise to the reaction system in an ice-water bath. The reaction solution was reacted for 1 h under normal temperature. The solvent was removed by concentration, and the crude product was purified by HPLC to obtain Compound 11. LCMS (M+H)⁺=511.0. ¹H NMR (400 MHz, DMSO) δ 8.66 (d, J=8.4 Hz, 1H), 8.54 (s, 1H), 8.08 (d, J=7.9 Hz, 1H), 7.37 (m, 3H), 7.03 (d, J=4.6 Hz, 1H), 6.62 (d, J=4.6 Hz, 1H), 4.26 (q, J=6.9 Hz, 2H), 4.09 (s, 1H), 3.79 (d, J=10.6 Hz, 3H), 1.99 (m, 2H), 1.79 (m, 2H), 1.67 (d, J=11.6 Hz, 1H), 1.45 (t, J=6.9 Hz, 3H), 1.38 (d, J=5.1 Hz, 4H), 1.19 (m, 1H).

### Example 12

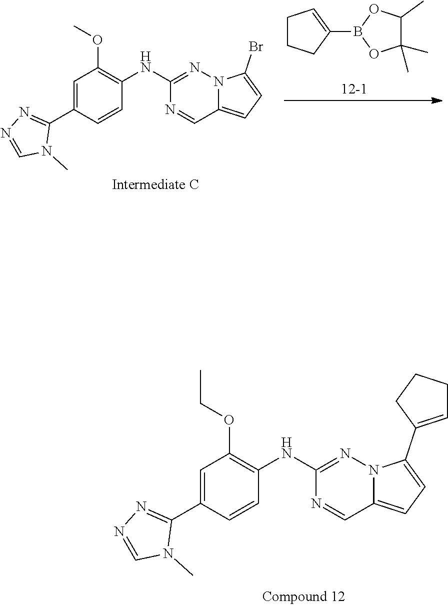

Step I:

Intermediate C (15 mg, 0.04 mmol) was dissolved in a mixture of dioxane (2 mL) and water (0.5 mL), and then Compound 12-1 (14 mg, 0.07 mmol), potassium carbonate (10 mg, 0.07 mmol) and Pd(dppf)Cl₂ (3 mg, 0.004 mmol) were added. The reaction solution was heated to 100° C. and reacted for 2 h under nitrogen atmosphere. The reaction solution was concentrated to obtain a crude product, and the crude product was separated by HPLC to obtain Compound 12. LCMS: (M+H)⁺=402.0. ¹H NMR (400 MHz, DMSO) δ 8.98 (s, 1H), 8.67 (s, 1H), 8.36-8.29 (m, 1H), 7.99 (s, 1H), 7.38 (dd, J=10.3, 1.9 Hz, 2H), 6.99 (s, 1H), 6.93 (d, J=4.7 Hz, 1H), 6.85 (d, J=4.7 Hz, 1H), 4.22 (q, J=6.9 Hz, 2H), 3.80 (s, 3H), 2.83 (s, 2H), 2.65 (d, J=16.7 Hz, 2H), 2.03-1.88 (m, 2H), 1.42 (t, J=6.9 Hz, 3H).

### Example 13

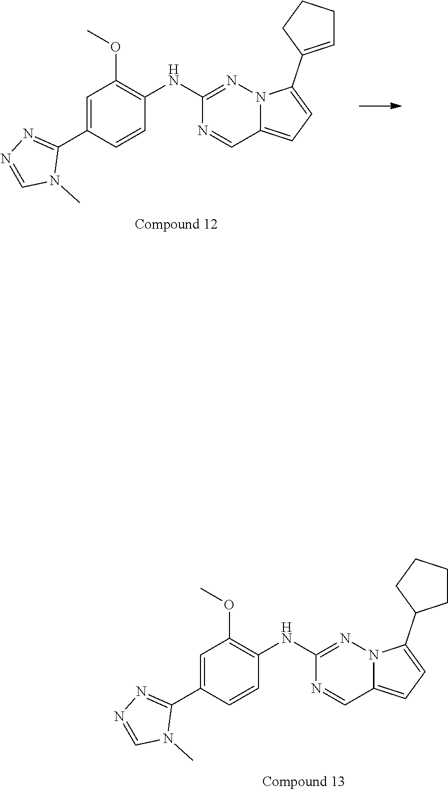 [SMILES: COc1cc(-c2nncn2C)ccc1Nc1ncc2ccc(C3=CCCC3)n2n1 ; COc1cc(-c2nncn2C)ccc1Nc1ncc2ccc(C3CCCC3)n2n1]

Step I:

Compound 12 (15 mg, 0.04 mmol) was dissolved in ethyl acetate (3 mL), and then 10% palladium on carbon (10 mg) was added and stirred for 5 h under hydrogen atmosphere at normal temperature. The reaction solution was diluted with ethyl acetate and filtered under suction. The filter cake was washed 3 times with ethyl acetate. The filtrate was combined and concentrated. Then the crude product was separated by HPLC to obtain Compound 13. LCMS: (M+H)⁺=404.0. ¹H NMR (400 MHz, DMSO) δ 8.89 (s, 1H), 8.61-8.40 (m, 2H), 7.77 (m, 1H), 7.44-7.30 (m, 2H), 6.86 (d, J=4.6 Hz, 1H), 6.72 (d, J=4.6 Hz, 1H), 4.24 (q, J=6.9 Hz, 2H), 3.78 (s, 3H), 3.57 (d, J=7.5 Hz, 1H), 2.17 (d, J=8.0 Hz, 2H), 1.86-1.64 (m, 6H), 1.45 (t, J=6.9 Hz, 3H).

### Example 14

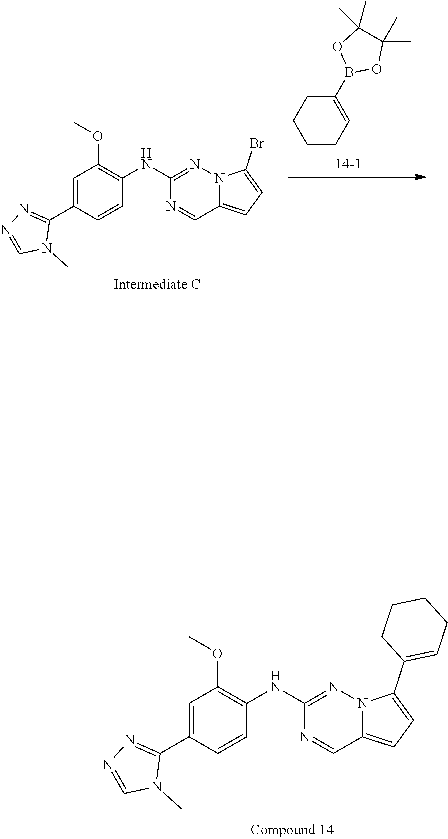 [SMILES: COc1cc(-c2nncn2C)ccc1Nc1ncc2ccc(Br)n2n1 ; CC1(C)OB(C2=CCCCC2)OC1(C)C ; COc1cc(-c2nncn2C)ccc1Nc1ncc2ccc(C3=CCCCC3)n2n1]

Step I:

Intermediate C (28 mg, 0.07 mmol) and Compound 14-1 (28 mg, 0.14 mmol) were dissolved in a mixture of dioxane (2 mL) and water (0.5 mL), and then potassium carbonate (19 mg, 0.14 mmol) and Pd(dppf)Cl₂ (10 mg, 0.014 mmol) were added to the reaction system. The reaction solution was heated to 90° C., and reacted for 18 h under nitrogen atmosphere. The reaction solution was concentrated under reduced pressure, and the crude product was separated by HPLC to obtain Compound 14. LCMS (M+H)⁺=416.0. ¹H NMR (400 MHz, DMSO) δ 8.94 (s, 1H), 8.57 (s, 1H), 8.35 (m, 1H), 7.87 (m, 1H), 7.37 (d, J=10.7 Hz, 2H), 7.12 (m, 1H), 6.89 (d, J=17.5 Hz, 2H), 4.24 (m, 2H), 3.78 (s, 3H), 2.01 (m, 1H), 1.73 (d, J=39.1 Hz, 5H), 1.43 (m, 3H), 1.24 (m, 2H).

### Example 15

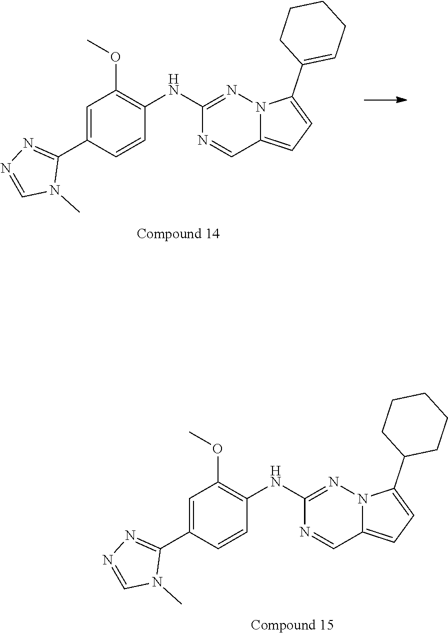 [SMILES: COc1cc(-c2nncn2C)ccc1Nc1ncc2ccc(C3=CCCCC3)n2n1 ; COc1cc(-c2nncn2C)ccc1Nc1ncc2ccc(C3CCCCC3)n2n1]

Step I:

Compound 14 (23 mg, 0.05 mmol) was dissolved in a mixture of MeOH (1 mL) and ethyl acetate (1 mL), and the reaction system was reacted for 2 h under hydrogen atmosphere at room temperature. The reaction solution was concentrated under reduced pressure, and the crude product was separated by HPLC to obtain Compound 15. LCMS: (M+H)⁺=418.0. ¹H NMR (400 MHz, DMSO) δ 8.89 (s, 1H), 8.50 (m, 2H), 7.78 (s, 1H), 7.36 (m, 2H), 6.86 (d, J=4.6 Hz, 1H), 6.67 (d, J=4.6 Hz, 1H), 4.25 (q, J=6.9 Hz, 2H), 3.78 (s, 3H), 3.15 (m, 1H), 2.09 (m, 2H), 1.86 (m, 2H), 1.52 (m, 2H), 1.44 (d, J=6.9 Hz, 3H), 1.30 (m, 2H), 1.23 (m, 2H).

### Example 16

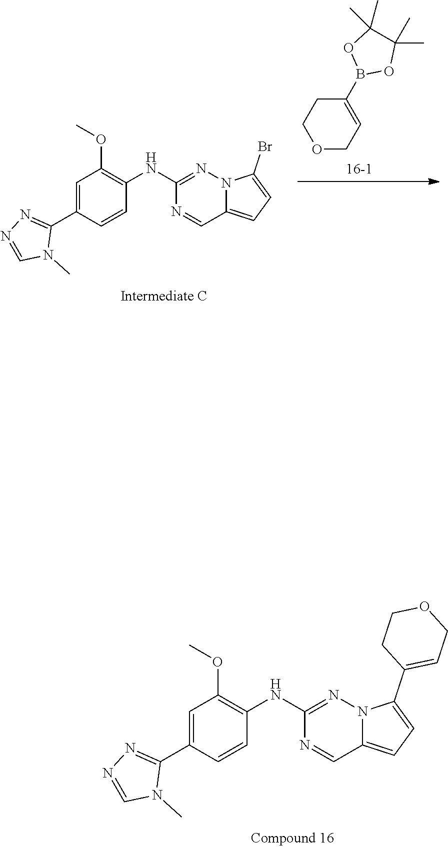 [SMILES: COc1cc(-c2nncn2C)ccc1Nc1ncc2ccc(Br)n2n1 ; CC1(C)OB(C2=CCOCC2)OC1(C)C ; COc1cc(-c2nncn2C)ccc1Nc1ncc2ccc(C3=CCOCC3)n2n1]

Step I:

Intermediate C (80 mg, 0.19 mmol), Compound 16-1 (80 mg, 0.38 mmol), Pd(dppf)Cl₂ (15 mg, 0.02 mmol) and K₂CO₃ (52 mg, 0.38 mmol) were dissolved in dioxane (6 mL) and H₂O (1 mL). The reaction system was heated to 110° C. and reacted for 8 h under nitrogen atmosphere. The reaction solution was concentrated under reduced pressure, and the crude product was separated by HPLC to obtain Compound 16. LCMS: (M+H)⁺=418.0. ¹H NMR (400 MHz, DMSO) δ 8.98 (s, 1H), 8.56 (s, 1H), 8.26 (d, J=8.1 Hz, 1H), 7.95 (s, 1H), 7.38 (m, 2H), 7.19 (m, 1H), 6.92 (dd, J=10.1, 4.8 Hz, 2H), 4.37 (d, J=2.6 Hz, 2H), 4.22 (q, J=6.9 Hz, 2H), 3.88 (t, J=5.4 Hz, 2H), 3.78 (s, 3H), 2.59 (m, 2H), 1.42 (t, J=6.9 Hz, 3H).

### Example 17

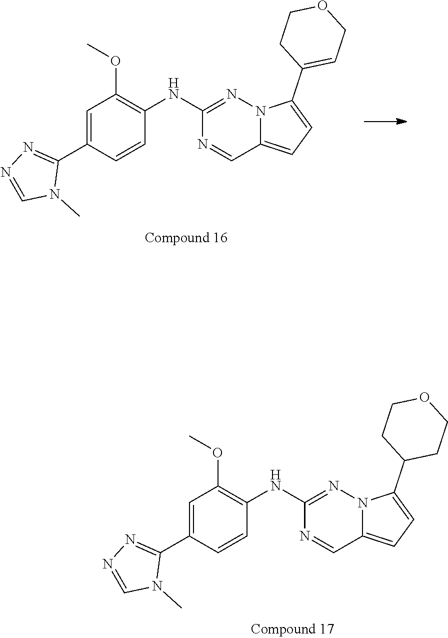 [SMILES: COc1cc(-c2nncn2C)ccc1Nc1ncc2ccc(C3=CCOCC3)n2n1 ; COc1cc(-c2nncn2C)ccc1Nc1ncc2ccc(C3CCOCC3)n2n1]

Step I:

Compound 16 (20 mg, 0.05 mmol) was dissolved in a mixture of MeOH (1 mL) and ethyl acetate (1 mL). The reaction system was reacted for 2 h under hydrogen atmosphere at room temperature. The reaction solution was concentrated under reduced pressure, and the crude product was separated by HPLC to obtain Compound 17. LCMS: (M+H)⁺=420.0. ¹H NMR (400 MHz, DMSO) δ 8.92 (s, 1H), 8.55 (s, 1H), 8.49 (d, J=8.8 Hz, 1H), 7.81 (s, 1H), 7.37 (m, 2H), 6.88 (d, J=4.6 Hz, 1H), 6.71 (d, J=4.6 Hz, 1H), 4.24 (q, J=7.0 Hz, 2H), 4.00 (dd, J=11.1, 3.0 Hz, 2H), 3.78 (s, 3H), 3.60 (dd, J=11.7, 9.9 Hz, 2H), 3.45 (m, 1H), 2.01 (d, J=14.9 Hz, 2H), 1.78 (dd, J=12.5, 3.9 Hz, 2H), 1.45 (m, 3H).

### Example 18

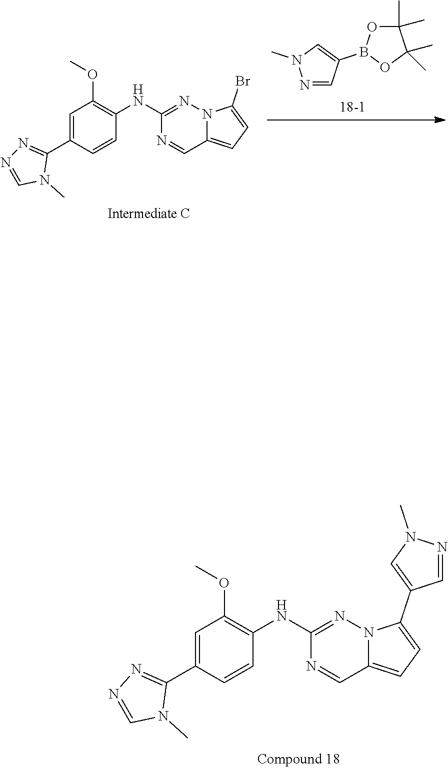

Step I:

Intermediate C (20 mg, 0.05 mmol) and Compound 18-1 (20 mg, 0.10 mmol) were dissolved in a mixture of dioxane (2 mL) and water (0.4 mL), and then potassium carbonate (14 mg, 0.1 mmol) and Pd(dppf)Cl₂ (5 mg, 0.005 mmol) were added to the reaction system. The reaction system was heated to 90° C. and reacted for 18 h under nitrogen atmosphere. The reaction solution was concentrated under reduced pressure, and the crude product was purified by HPLC to obtain Compound 18. LCMS (M+H)⁺=416.0. ¹H NMR (400 MHz, DMSO) δ 8.92 (s, 1H), 8.57 (s, 1H), 8.44 (s, 1H), 8.31 (d, J=8.1 Hz, 1H), 8.16 (s, 1H), 8.00 (s, 1H), 7.40 (m, 2H), 7.12 (d, J=4.7 Hz, 1H), 6.98 (d, J=4.7 Hz, 1H), 4.23 (q, J=6.9 Hz, 2H), 3.96 (s, 3H), 3.79 (s, 3H), 1.41 (t, J=6.9 Hz, 3H).

### Activity Test Examples

In the following examples, the TTK inhibitory activity and pharmacokinetic properties of the compound of the present disclosure are detected with some compounds of the present disclosure.

### Example A: TTK Inhibitory Activity

The experiment aims to detect the inhibitory activity of the compound of the present disclosure on TTK in vitro.

Experimental Steps and Methods:

The TTK kinase reaction system was 10 μL, including 0.5 nM TTK, test compound over a concentration gradient, 10 mM MgCl₂, 2 mM DTT, 7 uM ATP, 0.2 uM Fluorescein-PolyGT (Fluorescein-Poly Glu:Tyr (4:1)), 0.01% Triton X-100, 0.01% BSA, and 50 mM HEPES pH 7.5. The enzyme and the test compound of the present disclosure were added into a 384-well plate, and then a substrate and ATP were added. Then the system was incubated at a constant temperature of 28° C. 30 min after the reaction, 10 μL of a mixed solution of a corresponding antibody and EDTA was added to terminate the reaction, and incubated for 60 min at a constant temperature of 28° C. The data was read on Envision instrument. A curve was plotted with the Log concentration of the inhibitor as the X axis and the inhibition rate as the Y axis, and IC₅₀ was calculated by Y=Bottom+(Top-Bottom)/(1+(IC₅₀/X)\hat{ }HillSlope).

Table 1 shows the experimental data of the TTK inhibitory activity of some compounds of the present disclosure.

TABLE 1

| Compound No. | TTK IC₅₀ (nM) |
|---|---|
| Compound 1 | 27.0 |
| Compound 2 | 1.9 |
| Compound 3 | 8.9 |
| Compound 4 | 4.0 |
| Compound 5 | 1.7 |
| Compound 6 | 3.0 |
| Compound 7 | 1.1 |
| Compound 10 | 5.5 |
| Compound 12 | 8.7 |
| Compound 13 | 7.1 |
| Compound 14 | 2.4 |
| Compound 15 | 4.2 |
| Compound 16 | 2.0 |
| Compound 17 | 6.3 |
| Compound 18 | 2.1 |

The experimental results show that the compound of the present disclosure has good TTK inhibitory activity.

Pharmacokinetic Evaluation of the Compound of the Present Disclosure after Intravenous Injection or Oral Administration in Mice

This experiment aims to detect the pharmacokinetic properties of the compound of the present disclosure in mice.

Experimental Steps and Methods:

The test compound of the present disclosure was dissolved in 1000 DMSO/45% PEG400/45% water, whirled and ultrasonicated, to prepare a clear solution of a corresponding concentration, which was filtered through a microporous membrane filter for later use. Female Balb/c mice of 18-20 g were given the compound in solution by intravenous injection at a dose of is 1 mg/kg. The test compound was dissolved in 10% NMP/10% PEG-15-hydroxystearate/80% water, whirled and ultrasonicated, to prepare a clear solution of a corresponding concentration, which was filtered through a microporous membrane filter for later use. Female Balb/c mice of 18-20 g were given the compound in solution by oral administration at a dose of is 10 mg/kg. Whole blood was collected at certain time points to prepare plasma. The drug concentration was analyzed by LC-MS/MS, and the pharmacokinetic parameters were calculated by Phoenix WinNonlin software.

The experimental results show that the compound of the present disclosure has a large exposure and good absorption in the test animals, and the pharmacokinetic properties are obviously better.

In the description of the specification, the description with reference to the terms “an embodiment”, “some embodiments”, “example”, “specific example”, or “some example” and so on means that specific features, structures, materials or characteristics described in connection with the embodiment or example are embraced in at least one embodiment or example of the present disclosure. In the present specification, schematic representations of the above terms are not necessarily directed to the same embodiments or examples. Moreover, the described specific features, structures, materials or characteristics may be combined in any suitable manners in one or more embodiments. In addition, where there are no contradictions, the various embodiments or examples described in this specification and features of various embodiments or examples can be combined by those skilled in the art.

Although the embodiments of the present disclosure have been illustrated and described above, it is to be understood that the above embodiments are exemplary and not to be construed as limiting the present disclosure, and that changes, modifications, substitutions and alterations can be made by those skilled in the art without departing from the scope of the present disclosure.

What is claimed is:

1. A compound, which is a compound of Formula (I) or a stereoisomer, a tautomer, a N-oxide, a solvate, a metabolite, a pharmaceutically acceptable salt or a prodrug thereof: 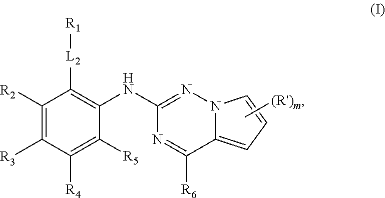
wherein
L₂ is a bond or O;
R₁, R₂, R₄, and R₅ are each independently H, F, Cl, Br, CN, NO₂, —OR^{b}, —NR^{c}R^{d}, or C₁₋₆ alkyl;
R₃ is —C(═O)R^{a}, —C(═O)OR^{b}, —S(═O)₂R^{b}, —C(═O)NR^{c}R^{d}, —OR^{b}, —NR^{c}R^{d}, R^{b}O—C₁₋₄ alkylene, R^{d}R^{c}N—C₁₋₄ alkylene, C₁₋₆ alkyl, C₂₋₆ alkenyl, C₂₋₆ alkynyl, C₃₋₈ cycloalkyl, C₃₋₈ cycloalkyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 10 atoms, or (heteroaryl having 5 to 10 atoms)-C₁₋₄ alkylene, in which the C₁₋₆ alkyl, C₂₋₆ alkenyl, C₂₋₆ alkynyl, C₃₋₈ cycloalkyl, C₃₋₈ cycloalkyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 10 atoms and (heteroaryl having 5 to 10 atoms)-C₁₋₄ alkylene are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene, or R^{d}R^{c}N—C₁₋₄ alkylene;
R⁶ is H or 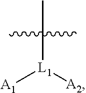
in which L₁ is N or O;
A₁ and A₂ are each independently H, C₁₋₆ alkyl, C₃₋₁₂ carbocyclyl, C₃₋₁₂ carbocyclyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 14 atoms, or (heteroaryl having 5 to 14 atoms)-C₁₋₄ alkylene, or A₁ and A₂, together with L₁ to which they are attached, form a heterocyclic ring having 3-6 atoms, in which the C₁₋₆ alkyl, C₃₋₁₂ carbocyclyl, C₃₋₁₂ carbocyclyl-C₁₋₄ alkylene, heterocyclyl having 3 to 12 atoms, (heterocyclyl having 3 to 12 atoms)-C₁₋₄ alkylene, C₆₋₁₀ aryl, C₆₋₁₀ aryl-C₁₋₄ alkylene, heteroaryl having 5 to 14 atoms, (heteroaryl having 5 to 14 atoms)-C₁₋₄ alkylene, or heterocyclic ring having 3-6 atoms formed by A₁ and A₂ together with L₁ to which they are attached are each independently unsubstituted or substituted with 1, 2, 3, 4 or 5 R′, provided that A₁ and A₂ are not both H;
each R′ is independently H, F, Cl, Br, CN, NO₂, ═O, —OR^{b}, —NR^{c}R^{d}, —S(═O)₂R^{b}, C₁₋₆ alkyl, C₁₋₆ haloalkyl, C₃₋₈ cycloalkyl, C₃₋₈ cycloalkenyl, heterocyclyl having 3 to 12 atoms, C₆₋₁₀ aryl or heteroaryl having 5 to 10 atoms, in which the C₃₋₈ cycloalkyl, heterocyclyl having 3 to 12 atoms, C₆₋₁₀ aryl or heteroaryl having 5 to 10 atoms are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene;
m is 0, 1, 2 or 3;
provided that when R₆ is H, m is not 0, and at least one R′ is C₃₋₈ cycloalkyl, C₃₋₈ cycloalkenyl, heterocyclyl having 3 to 12 atoms, C₆₋₁₀ aryl or heteroaryl having 5 to 10 atoms, in which the C₃₋₈ cycloalkyl, C₃₋₈ cycloalkenyl, heterocyclyl having 3 to 12 atoms, C₆₋₁₀ aryl heteroaryl having 5 to 10 atoms are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene; and
R^{a}, R^{b}, R^{c}, and R^{d} are each independently H, C₁₋₆ alkyl, C₁₋₆ haloalkyl, or heterocyclyl having 3 to 6 atoms, or R^{c} and R^{d}, together with nitrogen to which they are attached, form a heterocyclic ring having 3 to 6 atoms, in which the C₁₋₆ alkyl and heterocyclic ring having 3 to 6 atoms are each independently unsubstituted or substituted with 1, 2, 3 or 4 substituents independently selected from F, Cl, CN, OH, NH₂, C₁₋₆ alkyl, C₁₋₆ haloalkyl, C₁₋₆ alkoxy or C₁₋₆ alkylamino.

2. The compound according to claim 1, having a structure of Formula (II): 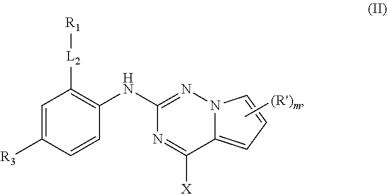
wherein X is represented by a sub-structural formula below: 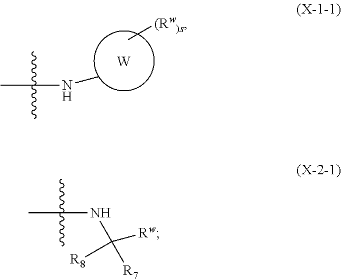
ring W is C₃₋₈ cycloalkyl, heterocyclic ring having 3 to 8 atoms, benzene, or heteroaryl ring having 5 to 6 atoms;
each R^{w} is independently H, F, Cl, Br, CN, NO₂, ═O, —OR^{b}, —NR^{c}R^{d}, —S(═O)₂R^{b}, C₁₋₆ alkyl or C₁₋₆ haloalkyl;
R₇ and R₈ are each independently H, or C₁₋₆ alkyl; and
s is 0, 1, 2 or 3.

3. The compound according to claim 1, having a structure of Formula (III): 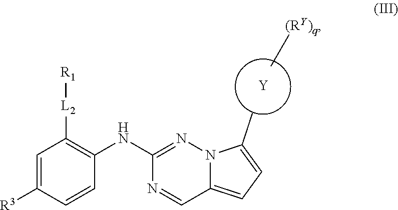
wherein Y is C₃₋₈ cycloalkyl, C₃₋₈ cycloalkenyl, heterocyclic ring having 3 to 8 atoms, benzene or heteroaryl ring having 5 to 6 atoms;
each R^{Y} is independently H, F, Cl, Br, CN, NO₂, ═O, —OR^{b}, —NR^{c}R^{d}, —S(═O)₂R^{b}, C₁₋₆ alkyl or C₁₋₆ haloalkyl; and
q is 0, 1, 2 or 3.

4. The compound according to claim 1, wherein R₂, R₄, and R₅ are each independently H.

5. The compound according to claim 1, wherein R₃ is —C(═O)NR^{c}R^{d}, OR^{b}, —NR^{c}R^{d}, —S(═O)₂R^{b}, heterocyclyl having 3 to 6 atoms, (heterocyclyl having 3 to 6 atoms)-C₁₋₄ alkylene, C₆₋₉ aryl, C₆₋₉ aryl-C₁₋₄ alkylene, heteroaryl having 5 to 9 atoms or (heteroaryl having 5 to 9 atoms)-C₁₋₄ alkylene.

6. The compound according to claim 1, wherein A₁ and A₂ are each independently H, C₁₋₆ alkyl, C₃₋₆ carbocyclyl, C₃₋₆ carbocyclyl-C₁₋₄ alkylene, heterocyclyl having 3 to 6 atoms, (heterocyclyl having 3 to 6 atoms)-C₁₋₄ alkylene, C₆₋₈ aryl, C₆₋₈ aryl-C₁₋₄ alkylene, heteroaryl having 5 to 8 atoms, or (heteroaryl having 5 to 8 atoms)-C₁₋₄ alkylene, or A₁ and A₂, together with L₁ to which they are attached, form a heterocyclic ring having 3 to 6 atoms.

7. The compound according to claim 1, wherein each R′ is independently H, F, Cl, Br, CN, NO₂, ═O, —OR^{b}, —NR_{c}R_{d}, —S(═O)OR^{b}, C₁₋₃ alkyl, C₁₋₃ haloalkyl, C₃₋₆ cycloalkyl, heterocyclyl having 3 to 6 atoms, C₆₋₈ aryl or heteroaryl having 5 to 8 atoms, in which the C₃₋₆ cycloalkyl, heterocyclyl having 3 to 6 atoms, C₆₋₈ aryl or heteroaryl having 5 to 8 atoms are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene.

8. The compound according to claim 1, wherein R^{a}, R^{b}, R^{c}, and R^{d} are each independently H, methyl, ethyl, isopropyl, n-propyl, n-butyl, t-butyl, C₁₋₃ haloalkyl, or heterocyclyl having 3 to 6 atoms, or R^{c} and R^{d}, together with nitrogen to which they are attached, form a heterocyclic ring having 3 to 6 atoms.

9. The compound according to claim 1, wherein A₁ and A₂ are each independently H, C₁₋₆ alkyl, C₃₋₆ carbocyclyl, heterocyclyl having 3 to 6 atoms, C₆₋₈ aryl, or heteroaryl having 5 to 8 atoms.

10. The compound according to claim 1, wherein R₃ is —C(═O)NR^{c}R^{d}, OR^{b}, —NR^{c}R^{d}, —S(═O)₂R^{b}, heterocyclyl having 3 to 6 atoms, phenyl, naphthyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, pyrimidinyl, indolyl, purinyl, quinolyl, isoquinolyl, or phenoxathiinyl, in which the heterocyclyl having 3 to 6 atoms, phenyl, naphthyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, pyrimidinyl, indolyl, purinyl, quinolyl, isoquinolyl, or phenoxathiinyl are each independently unsubstituted or substituted with 1, 2, 3 or 4 substituents independently selected from F, Cl, Br, CN, ═O, —OR^{b}, —NR^{c}R^{d}, C₁₋₆ alkyl, C₁₋₆ haloalkyl, R^{b}O—C₁₋₄ alkylene or R^{d}R^{c}N—C₁₋₄ alkylene.

11. The compound according to claim 2, wherein ring W is C₃₋₆ cycloalkyl, heterocyclyl having 3 to 6 atoms, phenyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, or pyrimidinyl.

12. The compound according to claim 2, wherein R^{w} is independently H, F, Cl, Br, CN, NO₂, ═O, —OR^{b}, —NR^{c}R^{d}, —S(═O)₂R^{b}, methyl, ethyl, isopropyl, n-propyl, n-butyl or t-butyl or C₁₋₆ haloalkyl.

13. The compound according to claim 3, wherein ring Y is C₃₋₆ cycloalkyl, C₃₋₆ cycloalkenyl, heterocyclyl having 3 to 6 atoms, phenyl, pyrrolyl, pyridinyl, pyrazolyl, imidazolyl, triazolyl, tetrazolyl, oxazolyl, oxadiazolyl, 1,3,5-triazinyl, thiazolyl, thienyl, pyrazinyl, pyridazinyl, pyrimidinyl, 3,6-dihydro-2H-pyran or tetrahydro-2H-pyran.

14. The compound according to claim 3, wherein R^{Y} is independently H, F, Cl, Br, CN, NO₂, ═O, —OR^{b}, —NR^{c}R^{d}, —S(═O)₂R^{b}, methyl, ethyl, isopropyl, n-propyl, n-butyl or t-butyl or C₁₋₆ haloalkyl.

15. The compound according to claim 2, having a structure of Formula (IV), (V), (VI), (VII), (VIII) or (VIIII): 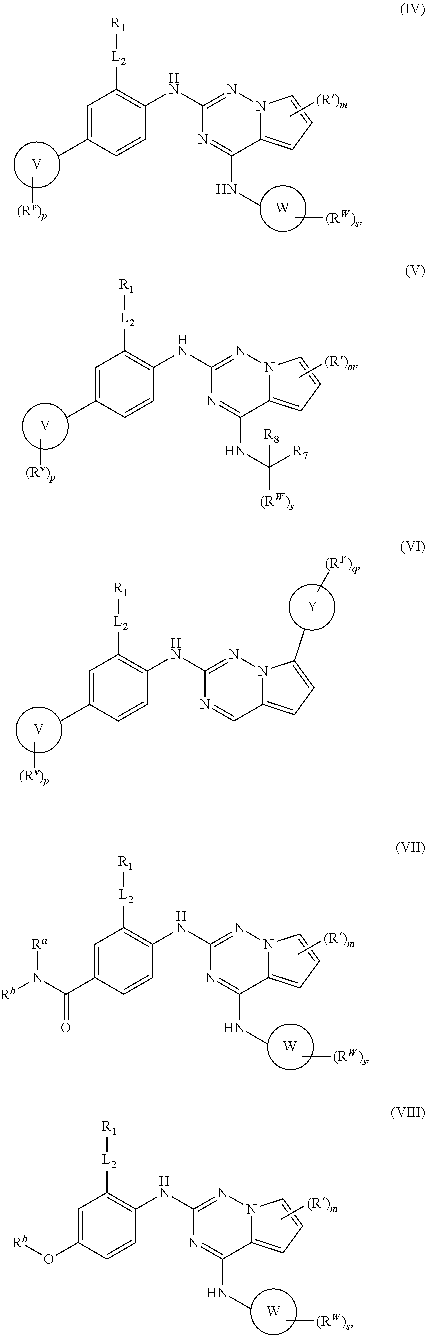 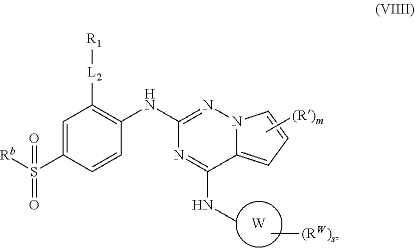
wherein V is C₃₋₈ cycloalkyl, heterocyclic ring having 3 to 8 atoms, benzene or heteroaryl ring having 5 to 6 atoms;
R^{V} is F, Cl, Br, CN, —OH, ═O, C₁₋₆ alkyl or C₁₋₆ haloalkyl; and
p is 0, 1, 2 or 3.

16. A compound, having one of the following structures: 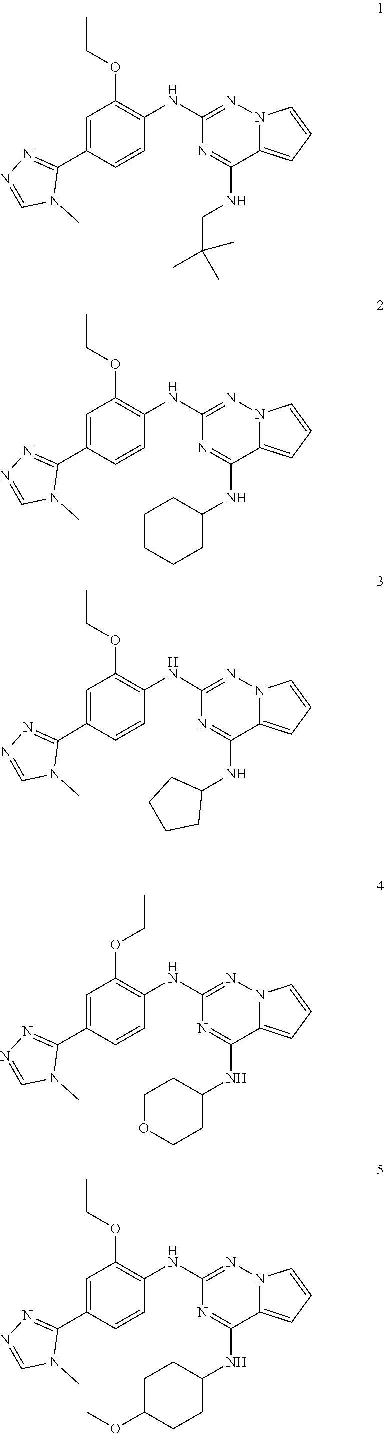 [SMILES: CCOc1cc(-c2nncn2C)ccc1Nc1nc(NCC(C)(C)C)c2cccn2n1 ; CCOc1cc(-c2nncn2C)ccc1Nc1nc(NC2CCCCC2)c2cccn2n1 ; CCOc1cc(-c2nncn2C)ccc1Nc1nc(NC2CCCC2)c2cccn2n1 ; CCOc1cc(-c2nncn2C)ccc1Nc1nc(NC2CCOCC2)c2cccn2n1 ; CCOc1cc(-c2nncn2C)ccc1Nc1nc(NC2CCC(OC)CC2)c2cccn2n1] 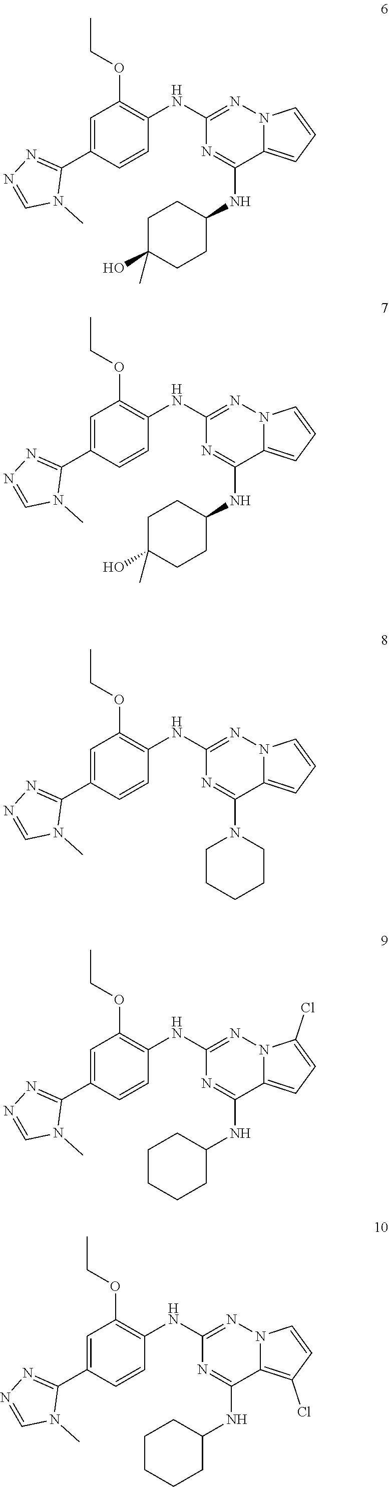 [SMILES: CCOc1cc(-c2nncn2C)ccc1Nc1nc(N[C@H]2CC[C@@](C)(O)CC2)c2cccn2n1 ; CCOc1cc(-c2nncn2C)ccc1Nc1nc(N[C@H]2CC[C@](C)(O)CC2)c2cccn2n1 ; CCOc1cc(-c2nncn2C)ccc1Nc1nc(N2CCCCC2)c2cccn2n1 ; CCOc1cc(-c2nncn2C)ccc1Nc1nc(NC2CCCCC2)c2ccc(Cl)n2n1 ; CCOc1cc(-c2nncn2C)ccc1Nc1nc(NC2CCCCC2)c2c(Cl)ccn2n1] 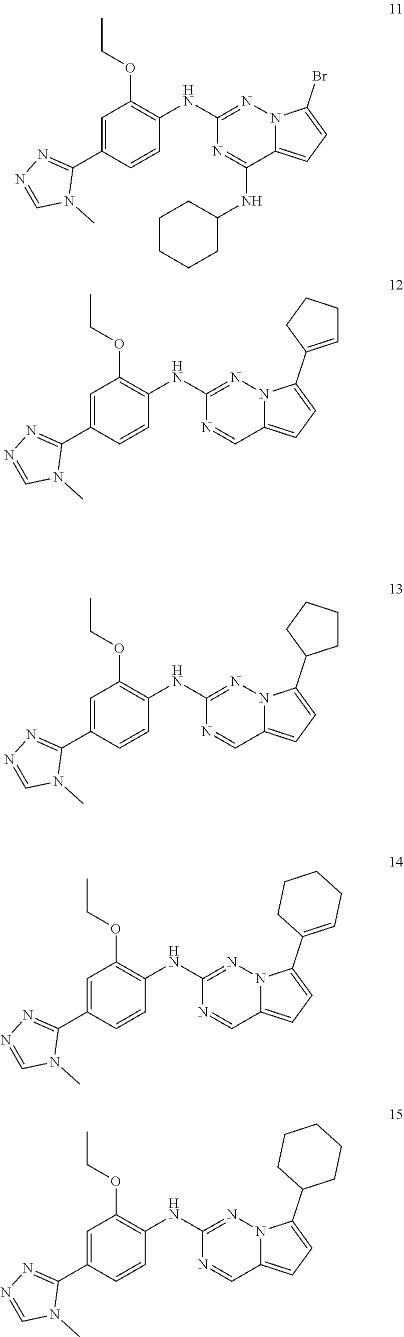 [SMILES: CCOc1cc(-c2nncn2C)ccc1Nc1nc(NC2CCCCC2)c2ccc(Br)n2n1 ; CCOc1cc(-c2nncn2C)ccc1Nc1ncc2ccc(C3=CCCC3)n2n1 ; CCOc1cc(-c2nncn2C)ccc1Nc1ncc2ccc(C3CCCC3)n2n1 ; CCOc1cc(-c2nncn2C)ccc1Nc1ncc2ccc(C3=CCCCC3)n2n1 ; CCOc1cc(-c2nncn2C)ccc1Nc1ncc2ccc(C3CCCCC3)n2n1] 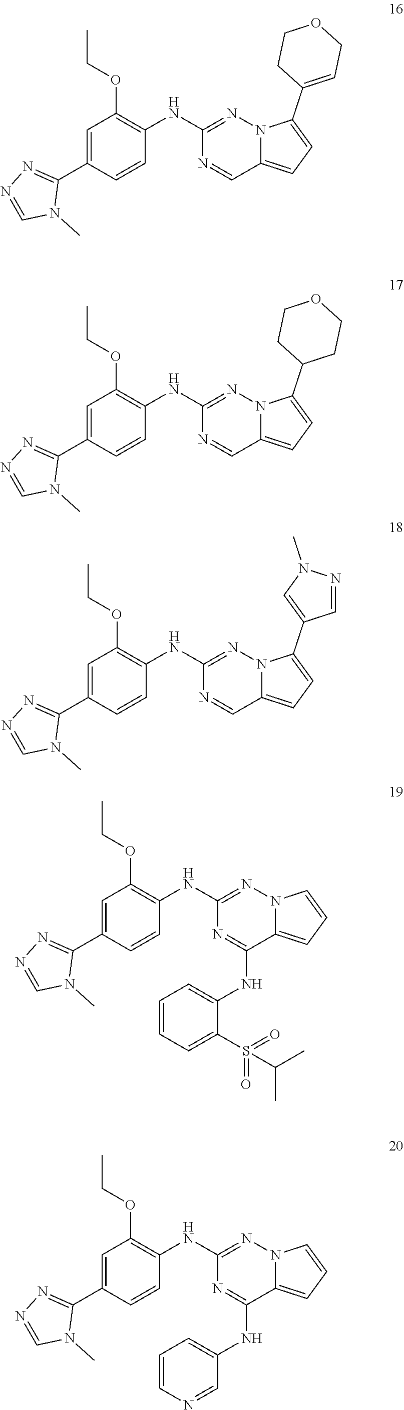 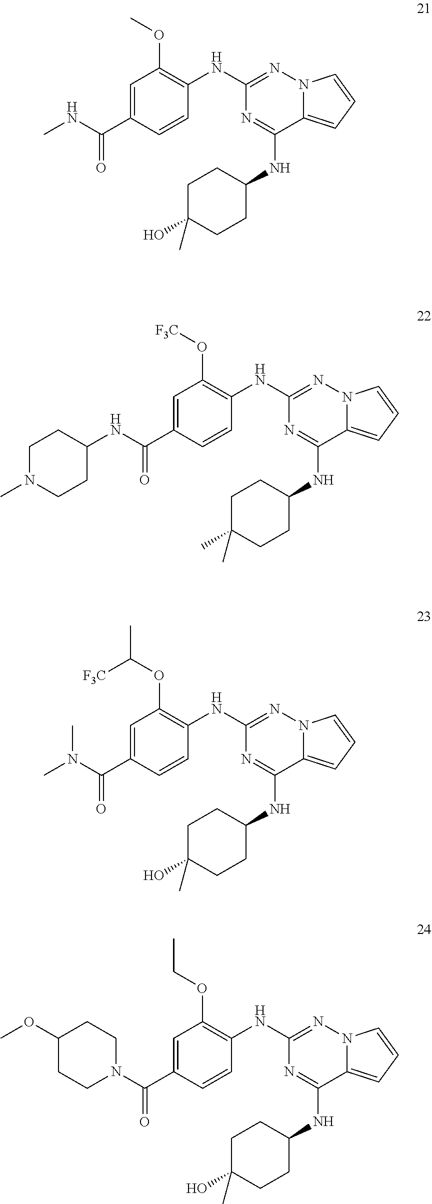 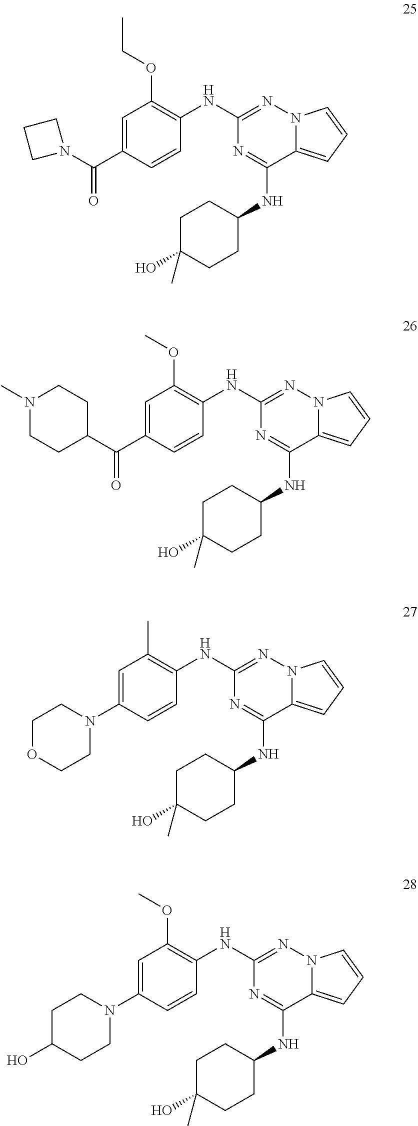 [SMILES: CCOc1cc(C(=O)N2CCC2)ccc1Nc1nc(N[C@H]2CC[C@](C)(O)CC2)c2cccn2n1 ; COc1cc(C(=O)C2CCN(C)CC2)ccc1Nc1nc(N[C@H]2CC[C@](C)(O)CC2)c2cccn2n1 ; Cc1cc(N2CCOCC2)ccc1Nc1nc(N[C@H]2CC[C@](C)(O)CC2)c2cccn2n1 ; COc1cc(N2CCC(O)CC2)ccc1Nc1nc(N[C@H]2CC[C@](C)(O)CC2)c2cccn2n1] 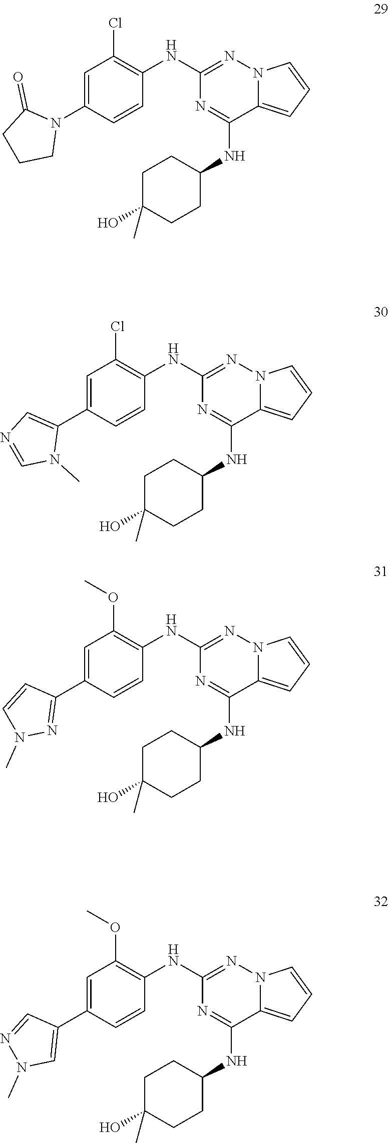 [SMILES: C[C@]1(O)CC[C@H](Nc2nc(Nc3ccc(N4CCCC4=O)cc3Cl)nn3cccc23)CC1 ; Cn1cncc1-c1ccc(Nc2nc(N[C@H]3CC[C@](C)(O)CC3)c3cccn3n2)c(Cl)c1 ; COc1cc(-c2ccn(C)n2)ccc1Nc1nc(N[C@H]2CC[C@](C)(O)CC2)c2cccn2n1 ; COc1cc(-c2cnn(C)c2)ccc1Nc1nc(N[C@H]2CC[C@](C)(O)CC2)c2cccn2n1] 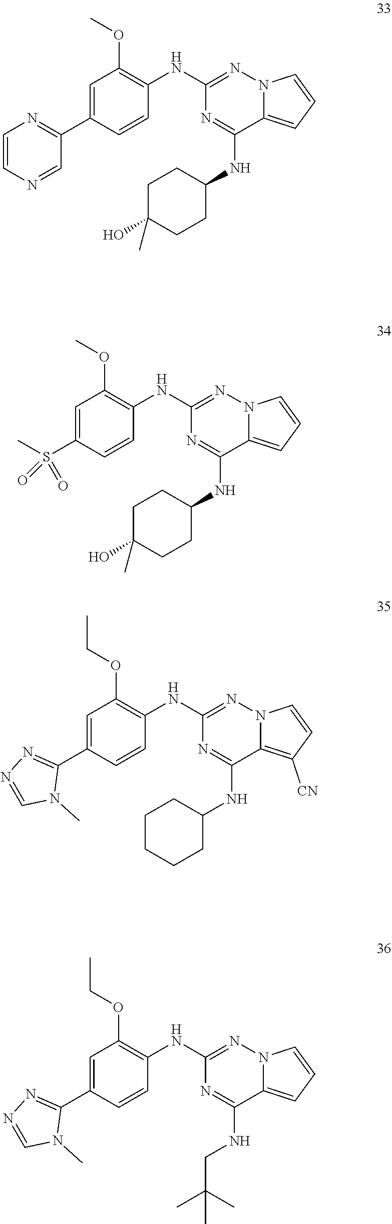 [SMILES: COc1cc(-c2cnccn2)ccc1Nc1nc(N[C@H]2CC[C@](C)(O)CC2)c2cccn2n1 ; COc1cc(S(C)(=O)=O)ccc1Nc1nc(N[C@H]2CC[C@](C)(O)CC2)c2cccn2n1 ; CCOc1cc(-c2nncn2C)ccc1Nc1nc(NC2CCCCC2)c2c(C#N)ccn2n1 ; CCOc1cc(-c2nncn2C)ccc1Nc1nc(NCC(C)(C)C)c2cccn2n1] 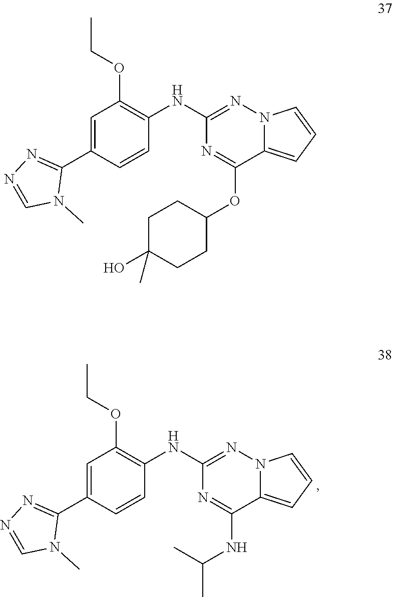 [SMILES: CCOc1cc(-c2nncn2C)ccc1Nc1nc(OC2CCC(C)(O)CC2)c2cccn2n1 ; CCOc1cc(-c2nncn2C)ccc1Nc1nc(NC(C)C)c2cccn2n1]
or a stereoisomer, a tautomer, a N-oxide, a solvate, a metabolite, a pharmaceutically acceptable salt or a prodrug thereof.

17. A pharmaceutical composition, comprising an effective amount of the compound according to claim 1.

18. The pharmaceutical composition according to claim 17, further comprising a pharmaceutically acceptable carrier, adjuvant, vehicle or a combination thereof.

19. The pharmaceutical composition according to claim 17, further comprising one or more therapeutic agents selected from other anti-tumor drugs.

20. The pharmaceutical composition according to claim 19, wherein the therapeutic agent is an antimitotic agent, an alkylating agent, an antimetabolic drug, a topoisomerase inhibitor, an estrogen receptor modulator, an androgen receptor modulator, a protein kinase targeting small molecule inhibitor, and a protein kinase targeting antibody drug.
